(19) United States
(12) Patent Application Publication
(10) Pub. No.: US 2024/0033218 A1
(43) Pub. Date: Feb. 1, 2024

(54) DIHYDROERGOTAMINE DRY POWDER FORMULATIONS AND METHODS OF USE

(71) Applicant: Pulmatrix Operating Company, Inc., Bedford, MA (US)

(72) Inventors: Jason M. PERRY, Somerville, MA (US); Aidan K. CURRAN, Duxbury, MA (US); Aaron C. GONZALEZ NELSON, Waltham, MA (US); Dayna F. HANSON, Natick, MA (US); Hillary T. BERGSON, Wilmington, MA (US); Eric C. STOLZ, Holden, MA (US)

(21) Appl. No.: 18/548,896

(22) PCT Filed: Mar. 1, 2022
(86) PCT No.: PCT/US2022/018306

§ 371 Date: Sep. 2, 2023

Related U.S. Application Data
(60) Provisional application No. 63/156,111, filed on Mar. 3, 2021.

(51) Int. Cl. A61K 9/00; A61K 31/5383; A61K 9/16

(52) U.S. Cl. CPC A61K 9/0075; A61K 31/5383; A61K 9/1623; A61K 9/1617

(57) ABSTRACT

The present disclosure relates to dry powder formulations comprising respirable dry particles that comprise 1) dihydroergotamine (DHE) or a salt, hydrate, or polymorph thereof, 2) a monovalent metal cation salt, and 3) one or more excipients; and also relates to methods of using the dry powder for the treatment of a migraine, headache, or a symptom thereof, methods of making the dry powder, and receptacles and devices containing the dry powder.

### RELATED APPLICATIONS

This application claims the benefit of U.S. Provisional Application No. 63/156,111, filed on Mar. 3, 2021, which is incorporated herein by reference in its entirety.

### BACKGROUND OF THE INVENTION

Dihydroergotamine (DHE) is an ergot alkaloid that is a safe and effective migraine treatment. (Silberstein, S., Headache (2020) 60(1):40-57). Despite this, DHE is not widely used due in part to its poor oral, sublingual, and intranasal bioavailability. (Saper, J. Headache (2006) 46 Suppl 4:S212-20). Consequently DHE is most often administered by injection in a clinical setting, which precludes patients from self-administering the drug. While nasal-spray formulations of DHE are available (e.g., MIGRANAL® and TRUDHESA™), they have many shortcomings such as having a slow onset, low bioavailability, variable or unpredictable effects in different patients, or causing an unpleasant taste, pain, cough, rhinitis, or pharyngitis, each of which can cause some patients to stop using the formulation. (Silberstein, supra; TRUDHESA™ [package insert]. Seattle, WA: Impel NeuroPharma Inc.).

A formulation of crystalline DHE suspended in propellant for oral inhalation was previously developed (MAP0004), however the FDA did not approve the product citing manufacturing, content uniformity and standards for device actuation issues (Silberstein, S., supra). Another shortcoming of propellant-based DHE formulations is the requirement for administration via a pressurized inhaler that must be shaken to mix the suspension, and then actuated using force, each requiring movements that can be difficult for patients to perform while suffering from a migraine. Further, pressurized inhalers typically require the user to coordinate their breathing maneuver with actuation of the inhaler to receive a complete dose (e.g., patients must actuate the device and inhale simultaneously), which can be particularly challenging for patients suffering from migraine, and for inhaler-naïve patients that are inexperienced with the use of inhalation devices. Additionally, pressurized inhalers often lead to inconsistent dosing and inefficient drug delivery.

While injectable DHE formulations (e.g., D.H.E. 45®) provide rapid and effective migraine treatment, they are typically only available in a clinical setting. The need to travel to the clinic during a migraine in order to obtain treatment is a significant barrier to access the drug. Moreover, patients typically experience a high incidence of side-effects following intravenous administration of DHE, such as emesis, nausea, and chest tightness. Other side-effects can include cardiovascular effects (e.g., blood pressure instability, arterial constriction, hypertension, or cardiac valvulopathy), paraesthesia, anxiety, dyspnea, headache, gastroparesis, diarrhea, skin rash, drowsiness, dizziness, flushing, increased sweating, retroperitoneal fibrosis, and pleural fibrosis (Silberstein, S., supra; Saper, J., supra; D.H.E. 45® [package insert]. Aliso Viejo, CA: Valeant Pharmaceuticals). Consequently, patients suffering a migraine are generally reluctant or unable to receive DHE.

As such, there is an unmet need for improved formulations of DHE that can be self-administered in a non-invasive way, to provide rapid and effective migraine relief with minimized side-effects.

### SUMMARY OF THE INVENTION

The invention relates to dry powder formulations comprising respirable dry particles that comprise dihydroergotamine (DHE) or a salt, hydrate, or polymorph thereof; a monovalent metal cation salt; and one or more excipients. In one aspect, the DHE or the salt, hydrate, or polymorph thereof is present in an amount of between about 1% to about 30% by weight of the dry particles. In a more particular aspect, the DHE or the salt, hydrate, or polymorph thereof is present in an amount of between about 1% to about 20% by weight of the dry particles. In an even more particular aspect, the DHE or the salt, hydrate, or polymorph thereof is present in an amount of between about 1% to about 15% by weight of the dry particles. In another particular aspect, the DHE or the salt, hydrate, or polymorph thereof is present in an amount of between about 1% by weight to about 10% by weight, of the dry particles. In some aspects, the DHE or the salt, hydrate, or polymorph thereof is DHE mesylate.

In some aspects, the dry powder comprises a first excipient and a second excipient, and the DHE or the salt, hydrate, or polymorph thereof is present in an amount of about 1% to about 30% by weight; the monovalent metal cation salt is present in an amount of about 2% to about 25% by weight; the first excipient is present in an amount of about 35% to about 75% by weight; and the second excipient is present in an amount of about 12% to about 25% by weight, wherein all percentages are weight percentages on a dry basis and all the components of the respirable dry particles amount to 100%. In another aspect, the dry powder comprises a first excipient and a second excipient, and the DHE or the salt, hydrate, or polymorph thereof is present in an amount of about 1% to about 25% by weight; the monovalent metal cation salt is present in an amount of about 4% to about 14% by weight; the first excipient is present in an amount of about 55% to about 75% by weight; and the second excipient is present in an amount of about 12% to about 25% by weight, wherein all percentages are weight percentages on a dry basis and all the components of the respirable dry particles amount to 100%. In a more particular aspect, the dry powder comprises a first excipient and a second excipient, and the DHE or the salt, hydrate, or polymorph thereof is present in an amount of about 10% by weight; the monovalent metal cation salt is present in an amount of about 9% by weight; the first excipient is present in an amount of about 63% by weight; and the second excipient is present in an amount of about 18% by weight, wherein all percentages are weight percentages on a dry basis and all the components of the respirable dry particles amount to 100%. In another particular aspect, the dry powder comprises a first excipient and a second excipient, and the DHE or the salt, hydrate, or polymorph thereof is present in an amount of about 3% by weight; the monovalent metal cation salt is present in an amount of about 9.7% by weight; the first excipient is present in an amount of about 67.9% by weight; and the second excipient is present in an amount of about 19.4% by weight, wherein all percentages are weight percentages on a dry basis and all the components of the respirable dry particles amount to 100%.

In some embodiments, the monovalent metal cation salt comprises a sodium salt, a potassium salt, or a lithium salt. In some embodiments, the monovalent metal cation salt comprises a sodium salt. In some embodiments, the monovalent metal cation salt comprises sodium chloride. In some embodiments, the monovalent metal cation salt comprises sodium sulfate.

The one or more excipients can be a sugar, a sugar alcohol, an oligosaccharide, an amino acid, or a combination thereof. In some embodiments, the excipient is a sugar alcohol or an amino acid. In some embodiments, the respirable dry particles comprise a combination of two excipients. In some embodiments, the respirable dry particles comprise a combination of mannitol and leucine.

In another aspect, the respirable dry particles comprise DHE mesylate, sodium chloride, leucine (e.g., L-leucine), and mannitol, wherein the DHE mesylate is present in an amount of about 1% to about 30% by weight; the sodium chloride is present in an amount of about 2% to about 25% by weight; the mannitol is present in an amount of about 35% to about 75% by weight; and the leucine is present in an amount of about 5% to about 35% by weight; wherein all percentages are weight percentages on a dry basis and all the components of the respirable dry particles amount to 100%. In a particular aspect, the respirable dry particles comprise DHE mesylate, sodium chloride, leucine (e.g., L-leucine), and mannitol, wherein the DHE mesylate is present in an amount of about 1% to about 15% by weight; the sodium chloride is present in an amount of about 4% to about 14% by weight; the mannitol is present in an amount of about 55% to about 75% by weight; and the leucine is present in an amount of about 12% to about 25% by weight; wherein all percentages are weight percentages on a dry basis and all the components of the respirable dry particles amount to 100%. In a more particular aspect, the respirable dry particles comprise DHE mesylate, sodium chloride, leucine (e.g., L-leucine), and mannitol, wherein the DHE mesylate is present in an amount of about 10% by weight; the sodium chloride is present in an amount of about 9% by weight; the mannitol is present in an amount of about 63% by weight; and the leucine is present in an amount of about 18% by weight; wherein all percentages are weight percentages on a dry basis and all the components of the respirable dry particles amount to 100%. In another particular aspect, the respirable dry particles comprise DHE mesylate, sodium chloride, leucine (e.g., L-leucine), and mannitol, wherein the DHE mesylate is present in an amount of about 3% by weight; the sodium chloride is present in an amount of about 9.7% by weight; the mannitol is present in an amount of about 67.9% by weight; and the leucine is present in an amount of about 19.4% by weight; wherein all percentages are weight percentages on a dry basis and all the components of the respirable dry particles amount to 100%. In some embodiments, the respirable dry powder comprises a dry powder formulation in Table 1.

The DHE or the salt, hydrate, or polymorph thereof (e.g., DHE mesylate) can be in any desired form, such as amorphous, crystalline, or a mixture of amorphous and crystalline. In some embodiments, the DHE or the salt, hydrate, or polymorph thereof (e.g., DHE mesylate) is amorphous. In some embodiments, the DHE or the salt, hydrate, or polymorph thereof (e.g., DHE mesylate) is crystalline.

Administering an effective amount of the dry powder to a subject in need thereof may result in a peak plasma concentration (Cₘₐₓ) of DHE of between about 1000 pg/mL and about 14,000 pg/mL, e.g., between about 2000 pg/mL and about 4,000 pg/mL, between about 4000 pg/mL and about 6,000 pg/mL, between about 6000 pg/mL and about 8,000 pg/mL, between about 8000 pg/mL and about 10,000 pg/mL, between about 10,000 pg/mL and about 12,000 pg/mL, or between about 12,000 pg/mL and about 14,000 pg/mL.

In some aspects, administering an effective amount of the dry powder to a subject in need thereof results in a peak plasma concentration (Cₘₐₓ) of DHE of between about 2000 pg/mL and about 6000 pg/mL. In more particular aspects, administering an effective amount of the dry powder to a subject in need thereof results in a Cₘₐₓ of DHE of between about 3000 pg/mL and about 5000 pg/mL. In some embodiments, administering an effective amount of the dry powder to a subject in need thereof results in a time to peak plasma concentration (Tₘₐₓ) of DHE of less than about 20 minutes (e.g., about 15 minutes, about 12 minutes, about 10 minutes, about 8 minutes, about 6 minutes, about 5 minutes, about 4 minutes, about 3 minutes, about 2 minutes, about 1 minute, or less). In some embodiments, administering an effective amount of the dry powder to a subject in need thereof results in an elimination half-life (t_{1/2}) of DHE of between about 6 hours and about 14 hours (e.g., between about 8 hours and about 12 hours). In some aspects, administering an effective amount of the dry powder to a subject in need thereof results in an AUC_{0-inf} of about 5000 pg*h/mL to about 10,000 pg*h/mL. In a more particular aspect, administering an effective amount of the dry powder to a subject in need thereof results in an AUC_{0-inf} of between about 7000 pg*h/mL and about 9000 pg*h/mL. In some embodiments, administering an effective amount of the dry powder to a subject in need thereof results in an AUC₀₋₄₈ₕ of about 4500 pg*h/mL to about 9500 pg*h/mL.

The dry powders disclosed herein may be administered to a subject repeatedly (e.g., daily) over a period of time and achieve a DHE accumulation ratio of less than 1.5, e.g., less than 1.4, less than 1.3, less than 1.2, less than 1.1, less than 1.0, less than 0.9, or less than 0.8. Without wishing to be bound by theory, an accumulation ratio of 1.5 or less is indicative of no DHE accumulation. The accumulation ratio can be calculated based on the ratio of an AUC recorded at a time point during a period of dosing, to an AUC recorded at the beginning of the dosing period. For example, the accumulation ratio for DHE over a daily dosing period of 14 days may be calculated as follows: (Day 14 AUC_{0-inf}/Day 1 AUC_{0-inf})=accumulation ratio.

The dry powder can consist of respirable dry particles that comprise DHE mesylate, sodium chloride, leucine (e.g., L-leucine), and mannitol. In some embodiments, the respirable dry particles consist of DHE mesylate, sodium chloride, leucine (e.g., L-leucine), and mannitol.

In some embodiments, the respirable dry particles have a volume median geometric diameter (VMGD) of about 10 microns or less (e.g., about 5 microns or less). In more particular aspects, the respirable dry particles have a VMGD of about 5 microns or less. In some embodiments, the respirable dry particles have a dispersibility ratio (1 bar/4 bar) of less than about 1.5 as measured by laser diffraction (RODOS/HELOS system). In some embodiments, the respirable dry powder has (i) a Fine Particle Fraction (FPF) of less than 5.6 microns of at least 45%; (ii) a FPF of less than 3.4 microns of at least 30%; or a FPF of less than 5.0 microns of at least 45%. In some embodiments, the dry powder has a mass median aerodynamic diameter (MMAD) of between about 1 micron and about 5 microns. In some embodiments, the respirable dry particles have a tap density of between about 0.1 g/cc and 1.0 g/cc. In some embodiments, the respirable dry particles have a tap density of between about 0.2 g/cc and 1.0 g/cc.

This disclosure also relates to methods for treating a migraine or a symptom thereof, by administering a dry powder described herein to a subject in need thereof by inhalation. In more preferred aspects, the dry powder is administered to the subject via oral inhalation. The dry powder can be administered to the subject at any point during a migraine (e.g., during the prodrome, aura, headache, or postdrome phase of the migraine). This disclosure also relates to the use of a dry powder as described herein for treating a migraine or a symptom thereof, and to a dry powder as described herein for use in the manufacture of a medicament for treating migraine or a symptom thereof.

In some embodiments, the incidence or severity of a side-effect (e.g., emesis) caused by administering an effective amount of the dry powder containing the DHE or a salt, hydrate, or polymorph thereof is reduced relative to the incidence or severity of the side effect following administration of an effective amount of DHE intravenously.

In some aspects, the treatment of the migraine provides relief of one or more migraine symptoms (e.g., pain, nausea, phonophobia, or photophobia). The relief of the migraine or a symptom thereof can be achieved within 30 minutes or less following administration of the dry powder.

The Cₘₐₓ of DHE in the subject after administering the dry powder may be between about 1000 pg/mL and about 14,000 pg/mL, e.g., between about 2000 pg/mL and about 4,000 pg/mL, between about 4000 pg/mL and about 6,000 pg/mL, between about 6000 pg/mL and about 8,000 pg/mL, between about 8000 pg/mL and about 10,000 pg/mL, between about 10,000 pg/mL and about 12,000 pg/mL, or between about 12,000 pg/mL and about 14,000 pg/mL.

In some embodiments, the Cₘₐₓ of DHE in the subject after administering the dry powder is between about 2000 pg/mL and about 6000 pg/mL. In a more particular aspect, the Cₘₐₓ of DHE in the subject after administering the dry powder is between about 3000 pg/mL and about 5000 pg/mL. In some embodiments, the Tₘₐₓ of DHE in the subject after administering the dry powder is less than about 20 minutes (e.g., about 15 minutes, about 12 minutes, about 10 minutes, about 8 minutes, about 6 minutes, about 5 minutes, about 4 minutes, about 3 minutes, about 2 minutes, about 1 minute, or less). In some embodiments, the t_{1/2} of DHE in the subject after administering the dry powder is between about 6 hours and about 14 hours (e.g., between about 8 hours and about 12 hours). In some embodiments, administering the dry powder to the subject results in an AUC_{0-inf} of between about 5000 pg*h/mL to about 10,000 pg*h/mL. In a more particular aspect, administering the dry powder to the subject results in an AUC_{0-inf} of between about 7000 pg*h/mL and about 9000 pg*h/mL. In some embodiments, administering the dry powder to the subject results in an AUC₀₋₄₈ₕ of between about 4500 pg*h/mL to about 9500 pg*h/mL.

In some embodiments, a total dose of DHE or a salt, hydrate, or polymorph thereof (e.g., DHE mesylate), of about 0.5 mg to about 2.0 mg is administered to the subject. In one particular aspect, a total dose of DHE or a salt, hydrate, or polymorph thereof (e.g., DHE mesylate), of between about 0.7 mg to about 1.5 mg is administered to the subject. In a more particular aspect, a total dose of DHE or a salt, hydrate, or polymorph thereof (e.g., DHE mesylate), of about 1.0 mg is administered to the subject.

This disclosure also relates to receptacles comprising a dry powder described herein. The receptacle can contain about 20 mg of the dry powder or less, e.g., about 10 mg or less, or about 5 mg or less, e.g., between about 1 mg and about 20 mg, between about 1 mg and about 10 mg, between about 1 mg and about 5 mg, between about 5 mg and about 10 mg, between about 10 mg and about 20 mg, between about 10 mg and about 15 mg, between about 15 mg and about 20 mg, e.g., about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, or about 20 mg, of the dry powder. In a more particular aspect, the receptacle contains between about 4 mg and about 6 mg of the dry powder.

The receptacle can contain a nominal dose of DHE or a salt, hydrate, or polymorph thereof (e.g., DHE mesylate) of between about 100 μg and about 2000 μg, e.g., between about 100 μg and about 1500 μg, between about 100 μg and about 1000 μg, between about 500 μg and about 2000 μg, between about 500 μg and about 1500 μg, e.g., about 100 μg, about 150 μg, about 200 μg, about 250 μg, about 500 μg, about 750 μg, about 1000 μg, about 1250 μg, about 1500 μg, about 1750 μg, or about 2000 μg, of DHE or a salt, hydrate, or polymorph thereof. In a more particular aspect, the receptacle contains a nominal dose of DHE or a salt, hydrate, or polymorph thereof (e.g., DHE mesylate) of about 150 μg or about 500 μg.

The disclosure also relates to dry powder inhaler (DPI) that contains a dry powder described herein. In some embodiment, the DPI is a passive DPI. In some embodiment, the DPI is a capsule-based DPI (e.g., a passive capsule-based DPI). In some embodiment, the DPI is a blister-based DPI (e.g., a passive blister-based DPI). In some embodiments, the DPI is a reservoir-based DPI (e.g., a passive reservoir-based DPI).

### BRIEF DESCRIPTION OF THE DRAWINGS

FIG. 1 is a graph depicting the mean plasma concentration of dihydroergotamine (DHE) (ng/mL) over time (h) following administration via inhalation of Formulation I in a dog model at three exemplary dose levels.

FIG. 2 is a graph depicting the mean plasma concentration of dihydroergotamine (DHE) (ng/mL) over time (h) following administration via inhalation of Formulation II in a dog model at three exemplary dose levels.

FIG. 3 is a graph depicting the mean plasma concentration of dihydroergotamine (DHE) (ng/mL) over time (h) following administration via inhalation of Formulation III in a dog model at three exemplary dose levels.

FIG. 4 is a graph depicting the mean plasma concentration of dihydroergotamine (DHE) (ng/mL) over time (h) following administration via inhalation of Formulation IV in a dog model at three exemplary dose levels.

FIG. 5 is a graph depicting the modeled mean plasma concentration of dihydroergotamine (DHE) (ng/mL) over time (h) following administration via inhalation of MAP0004 in a dog model at three exemplary dose levels, based on modelling of published data (Armer et al. vide infra).

FIG. 6 is a graph depicting the plasma concentration of DHE over time, following administration of an exemplary dry powder in dogs at three different dose levels (250 μg/kg/day, 400 μg/kg/day, and 600 μg/kg/day) by inhalation, as recorded on Day 1 of a 14-day dosing period.

FIG. 7 is a graph depicting the plasma concentration of DHE over time, following administration of an exemplary dry powder in dogs at three different dose levels (250 μg/kg/day, 400 μg/kg/day, and 600 μg/kg/day) by inhalation, as recorded on Day 14 of a 14-day dosing period.

### DETAILED DESCRIPTION OF THE INVENTION

This disclosure relates to a respirable dry powder containing respirable dry particles that comprise dihydroergotamine (DHE) or a salt, hydrate, or polymorph thereof (e.g., DHE mesylate), a monovalent metal cation salt (e.g., sodium chloride), and one or more excipients (e.g., leucine and mannitol); in addition to methods of making and using the dry powder; and receptacles and devices containing the dry powder.

The dry powders disclosed herein provide several advantages. For example, the dry powders of the present disclosure can be administered in an effective amount to a subject in need thereof to provide rapid migraine relief with minimal side-effects, and is suitable for self-administration using a dry powder inhaler. The dry powders disclosed herein are highly dispersible and typically the dispersibility is flow rate independent, meaning that the dry powders disperse and deliver the desired does of DHE across a wide range of flow rates. Thus, the dry powders can be effectively administered using a passive device with a low level of inhalation force from the patient. This is a particular advantage for patients suffering from impairment due to migraine, who may have difficulty operating more complex devices. Further, the dry powders can be administered from a passive device (e.g., passive dry powder inhaler) that does not require the user to perform coordinated breathing and actuation maneuvers to use. This feature is advantageous for patients with a migraine, and inhaler-naïve patients, who may be unable to carry out the coordinated maneuver typically required by other types of devices, such as pressurized inhalers that require actuating and inhaling simultaneously. The dry powders described herein are also typically uniform and consist of a single type of dry particle that includes DHE and all excipients, which provides for dose uniformity and consistent delivery of the desired dose. Therefore, the impaired patient is not required to mix or resuspend the drug to ensure that the proper dose of DHE is administered. Again, this type of manipulation can be difficult for patients that are impaired by migraine. The dry powders also provide a convenient and consistent dosage form for the self-administration of DHE in a non-invasive way, with minimal discomfort to the patient. Without wishing to be bound by theory, it is believed that administering the dry powder by inhalation results in a short time to peak plasma concentration (Tₘₐₓ) of DHE which contributes to a rapid therapeutic effect. Additionally, administering the dry powder by inhalation can produce a peak plasma concentration (Cₘₐₓ) effective to treat migraine and also minimize side effects of DHE such as emesis, and results in a sufficiently short elimination half-life (t_{1/2}) to suppress undesired side-effects without comprising efficacy.

In one aspect of the present disclosure, the dry powder comprises respirable dry particles that comprise DHE mesylate, sodium chloride, leucine (e.g., L-leucine), and mannitol. In a preferred aspect, the DHE mesylate is present in an amount of about 1% to about 25% by weight (e.g., about 1% to about 15%, e.g., about 3% or 10%); the sodium chloride is present in an amount of about 4% to about 14% by weight (e.g., about 9.0% or 9.7%); the mannitol is present in an amount of about 55% to about 75% by weight (e.g., about 63.0% or 67.9%); and the leucine is present in an amount of about 12% to about 25% by weight (e.g., about 18.0% or 19.4%); wherein all percentages are weight percentages on a dry basis and all the components of the respirable dry particles amount to 100%.

In another preferred aspect, the DHE mesylate is present in an amount of about 9% to about 11% by weight (e.g., 10%); the sodium chloride is present in an amount of about 8% to about 10% by weight (e.g., about 9%); the mannitol is present in an amount of about 62% to about 64% by weight (e.g., about 63%); and the leucine is present in an amount of about 17% to about 19% by weight (e.g., about 18%); wherein all percentages are weight percentages on a dry basis and all the components of the respirable dry particles amount to 100%

In yet another preferred aspect, the DHE mesylate is present in an amount of about 2% to about 4% by weight (e.g., 3%); the sodium chloride is present in an amount of about 8.7% to about 10.7% by weight (e.g., about 9.7%); the mannitol is present in an amount of about 66.9% to about 68.9% by weight (e.g., about 67.9%); and the leucine is present in an amount of about 18.4% to about 20.4% by weight (e.g., about 19.4%); wherein all percentages are weight percentages on a dry basis and all the components of the respirable dry particles amount to 100%.

These respirable dry powders comprising respirable dry particles may be manufactured from their components, in solutions or suspensions that are aqueous and/or contain another solvent, by spray drying or other suitable processes. The respirable dry powders comprising respirable dry particles are relatively dry in water and solvent content, small in geometric diameter, dense in mass density, and dispersible in that they deagglomerate from each other with a relatively low amount of energy. They have superior aerosol properties such as a relatively small aerodynamic diameter, a relatively high fine particle fraction and fine particle dose below sizes that are relevant to lung deposition. These properties are illustrated for exemplary dry powder formulations in Examples 1 and 2. Additionally, the respirable dry powders disclosed herein have advantageous pharmacokinetic properties, that can contribute to effective and rapid relief when administered to a subject in need thereof. The pharmacokinetic properties of exemplary dry powder formulations in a dog model are provided in Example 3.

### Definitions

As used herein, the term “about” refers to a relative range of plus or minus 5% of a stated value, e.g., “about 20 mg” would be “20 mg plus or minus 1 mg”.

As used herein, the term “administering” a dry powder or respirable dry particles refers to introducing the dry powder or respirable dry particles to the respiratory tract of a subject.

The term “AUC” as used herein refers to the area under the plasma concentration-time curve. AUC_{inf} refers to AUC from time 0 to infinity; AUCₗₐₛₜ refers to AUC from time 0 to time of last measurable concentration; AUC₀₋₂₄ₕ refers to AUC from time 0 to 24 hours; AUC₀₋₈ₕ refers to AUC from time 0 to 8 hours; AUC₀₋₄ₕ refers to AUC from time 0 to 4 hours.

The term “amorphous” as used herein indicates lack of significant crystallinity (i.e., less than about 5% crystallinity) when analyzed via powder X-ray diffraction (XRD).

The term “capsule emitted powder mass” or “CEPM” as used herein refers to the amount of dry powder formulation emitted from a capsule or dose unit container during an inhalation maneuver. CEPM can be measured gravimetrically, typically by weighing a capsule before and after the inhalation maneuver to determine the mass of powder formulation removed. CEPM can also be determined analytically, whereby the quantity of active substance remaining in the capsule is assayed via chromatographic methods (e.g. HPLC or UPLC) and then subtracted from the initial quantity of active substance in the capsule. CEPM can be expressed either as the mass of powder removed, in milligrams, or as a percentage of the initial filled powder mass in the capsule prior to the inhalation maneuver.

The term “dispersible” is a term of art that describes the characteristic of a dry powder or dry particles to be dispelled into a respirable aerosol. Dispersibility of a dry powder or dry particles is expressed herein as the quotient of the volume median geometric diameter (VMGD) measured at a dispersion (i.e., regulator) pressure of 1 bar divided by the VMGD measured at a dispersion (i.e., regulator) pressure of 4 bar, VMGD at 0.5 bar divided by the VMGD at 4 bar as measured by HELOS/RODOS, VMGD at 0.2 bar divided by the VMGD at 2 bar as measured by HELOS/RODOS, or VMGD at 0.2 bar divided by the VMGD at 4 bar as measured by HELOS/RODOS. These quotients are referred to herein as “1 bar/4 bar,” “0.5 bar/4 bar,” “0.2 bar/2 bar,” and “0.2 bar/4 bar,” respectively, and dispersibility correlates with a low quotient. For example, 1 bar/4 bar refers to the VMGD of respirable dry particles or powders emitted from the orifice of a RODOS dry powder disperser (or equivalent technique) at about 1 bar, as measured by a HELOS or other laser diffraction system, divided the VMGD of the same respirable dry particles or powders measured at 4 bar by HELOS/RODOS. Thus, a highly dispersible dry powder or dry particles will have a 1 bar/4 bar or 0.5 bar/4 bar ratio that is close to 1.0. Highly dispersible powders have a low tendency to agglomerate, aggregate or clump together and/or, if agglomerated, aggregated or clumped together, are easily dispersed or de-agglomerated as they emit from an inhaler and are breathed in by a subject. Dispersibility can also be assessed by measuring the size emitted from an inhaler as a function of flow rate. VMGD may also be called the volume median diameter (VIVID), x50, or Dv50.

The term “crystalline” as used herein indicates significant crystallinity (i.e., at least about 95% crystallinity) when analyzed via powder X-ray diffraction (XRD).

The term “dry powder” as used herein refers to a composition that contains finely dispersed respirable dry particles that are capable of being dispersed in an inhalation device and subsequently inhaled by a subject. Such a dry powder may contain up to about 25%, up to about 20%, or up to about 15% water or other solvent, or be substantially free of water or other solvent, or be anhydrous.

The term “dry particles” as used herein refers to respirable particles that may contain up to about 25%, up to about 20%, or up to about 15% water or other solvent, or be substantially free of water or other solvent, or be anhydrous.

The term “effective amount” as used herein refers to the amount of an active agent (e.g., DHE or a salt, hydrate, or polymorph thereof, such as DHE mesylate) or dry powder containing the active agent needed to achieve the desired therapeutic effect, such as an amount that is sufficient to treat a migraine or a symptom thereof, for example, relief of pain, photophobia and/or phonophobia, and/or produce an effective serum concentration of an active agent. The actual effective amount for a particular use can vary according to the particular dry powder or dry particle, the mode of administration, and the age, weight, general health of the subject, and severity of the symptoms or condition being treated. Suitable amounts of dry powders and dry particles to be administered, and dosage schedules for a particular subject can be determined by a clinician of ordinary skill based on these and other considerations.

The term “emitted dose” (ED) as used herein refers to an indication of the delivery of a drug formulation from a suitable inhaler device after a firing or dispersion event. More specifically, for dry powder formulations, the ED is a measure of the percentage of powder that is drawn out of a unit dose package and that exits the mouthpiece of an inhaler device. The ED is defined as the ratio of the dose delivered by an inhaler device to the nominal dose (i.e., the mass of powder per unit dose placed into a suitable inhaler device prior to firing). The ED is an experimentally-measured parameter, and can be determined using the method of USP Section 601 Aerosols, Metered-Dose Inhalers and Dry Powder Inhalers, Delivered-Dose Uniformity, Sampling the Delivered Dose from Dry Powder Inhalers, United States Pharmacopeia convention, Rockville, MD, 13th Revision, 222-225, 2007. This method utilizes an in vitro device set up to mimic patient dosing.

The term “fine particle fraction of less than 5.6 microns” (FPF (<5.6), or FPF (<5.6 microns)) as used herein, refers to the fraction of a sample of dry particles that have an aerodynamic diameter of less than 5.6 microns. For example, FPF (<5.6) can be determined by dividing the mass of respirable dry particles deposited on the stage one and on the collection filter of a two-stage collapsed Andersen Cascade Impactor (ACI) by the mass of respirable dry particles weighed into a capsule for delivery to the instrument. This parameter may also be identified as “FPF_TD (<5.6),” where TD means total dose. A similar measurement can be conducted using an eight-stage ACI. The eight-stage ACI cutoffs are different at the standard 60 L/min flow rate, but the FPF_TD (<5.6) can be extrapolated from the eight-stage complete data set. The eight-stage ACI result can also be calculated by the USP method of using the dose collected in the ACI instead of what was in the capsule to determine FPF.

The term “fine particle fraction of less than 5.0 microns” (FPF (<5.0), FPF<5 μm, or FPF (<5.0 microns)) and as used herein, refers to the fraction of a mass of respirable dry particles that have an aerodynamic diameter of less than 5.0 micrometers. For example, FPF (<5.0) can be determined by using an eight-stage ACI at the standard 60 L/min flow rate by extrapolating from the eight-stage complete data set. This parameter may also be identified as “FPF_TD (<5.0),” where TD means total dose. When used in conjunction with a geometric size distribution such as those given by a Malvern Spraytec, Malvern Mastersizer or Sympatec HELOS particle sizer, “FPF (<5.0)” refers to the fraction of a mass of respirable dry particles that have a geometric diameter of less than 5.0 micrometers.

The term “fine particle dose of less than 4.4 microns” (FPD (<4.4), FPD<4.4 μm, or FPD (<4.4 microns)) as used herein, refers to the mass of respirable dry powder particles that have an aerodynamic diameter of less than 4.4 micrometers. For example, FPD<4.4 μm can be determined by using an eight-stage ACI at the standard 60L/min flowrate and summing the mass deposited on the filter, and stages 6, 5, 4, 3, and 2 for a single dose of powder actuated into the ACI.

The term “fine particle fraction of less than 3.4 microns” (FPF (<3.4), FPF (<3.4 microns), as used herein, refer to the fraction of a mass of respirable dry particles that have an aerodynamic diameter of less than 3.4 microns. For example, FPF (<3.4) can be determined by dividing the mass of respirable dry particles deposited on the collection filter of a two-stage collapsed ACI by the total mass of respirable dry particles weighed into a capsule for delivery to the instrument. This parameter may also be identified as “FPF_TD (<3.4),” where TD means total dose. A similar measurement can be conducted using an eight-stage ACI. The eight-stage ACI result can also be calculated by the USP method of using the dose collected in the ACI instead of what was in the capsule to determine FPF.

The term “Hausner ratio” as used herein is a term of art that refers to the tap density divided by the bulk density and typically correlates with bulk powder flowability (i.e., an increase in the Hausner ratio typically corresponds to a decrease in powder flowability.

The term “respirable” as used herein refers to dry particles or dry powders that are suitable for delivery to the respiratory tract (e.g., pulmonary delivery) in a subject by inhalation. Respirable dry powders or dry particles have a mass median aerodynamic diameter (MMAD) of less than about 10 microns, preferably about 5 microns or less.

As used herein, the term “respiratory tract” includes the upper respiratory tract (e.g., nasal passages, nasal cavity, throat, and pharynx), respiratory airways (e.g., larynx, trachea, bronchi, and bronchioles) and lungs (e.g., respiratory bronchioles, alveolar ducts, alveolar sacs, and alveoli).

The term “small” as used herein to describe respirable dry particles refers to particles that have a volume median geometric diameter (VMGD) of about 10 microns or less, preferably about 5 microns or less. VMGD may also be called the volume median diameter (VIVID), x50, or Dv50.

All references to salts (e.g., sodium containing salts) herein include anhydrous forms and all hydrated forms of the salt.

All weight percentages are given on a dry basis.

### Dry Powders and Dry Particles

Aspects of the invention relate to respirable dry powders and dry particles that contain dihydroergotamine or a salt, hydrate, or polymorph thereof, in addition to a monovalent metal cation salt (e.g., a sodium salts and/or a potassium salt), and one or more excipients (e.g., leucine and mannitol).

### Dihydroergotamine

Dihydroergotamine (DHE) is a semi-synthetic ergot alkaloid having potent alpha-adrenergic antagonist activity, and is approved for use in the treatment of acute migraine, status migrainosus, and cluster headaches. DHE is also effective in treating triptan-resistant migraines, menstrual migraines, migraine with allodynia, severe or prolonged migraines, and cluster headaches. However, since the absorption of DHE from the gut is highly variable, and is prone to metabolism, DHE has poor oral bioavailability and non-oral routes of administration are necessary.

The respirable dry particles of the present disclosure can contain any desired amount, on a weight basis, of DHE or a salt, hydrate, or polymorph thereof, and typically contain about 1% to about 30% of DHE or a salt, hydrate, or polymorph thereof, by weight (wt %). In a preferred aspect, the respirable dry powders contain between about 1% to about 25% of DHE, or a salt, hydrate, or polymorph thereof (e.g., DHE mesylate), e.g., about 1%, about 2%, about 3%, about 4%, about 5%, about 6%, about 7%, about 8%, about 9%, about 10%, about 11%, about 12%, about 13%, about 14%, about 15%, about 20%, or about 25%, by weight. More preferably, the respirable dry particles contain between about 1% to about 10% by weight of DHE or a salt, hydrate, or polymorph thereof (e.g., about 1.5%, about 3.0%, about 5.8%, or about 10.0% by weight). In particular embodiments, the range of DHE or a salt, hydrate, or polymorph thereof is about 1% to about 3%, about 3% to about 5%, about 5% to about 7%, about 7% to about 9%, about 9% to about 11%, about 11 to about 13%, or about 13% to about 15% by weight. The amount of DHE or a salt, polymorph, or hydrate thereof in the respirable dry particles by weight is also referred to as the “drug load.”

It is preferred that the respirable dry particles contain an amount of DHE or a salt, hydrate, or polymorph thereof sufficient to provide therapeutically effective dose to a subject in need thereof, without the need for the subject to inhale large volumes of dry powder. For example, the dry particles can comprise an amount of DHE mesylate sufficient to provide a therapeutically effective dose to a subject by inhaling about 20 mg or less, about 15 mg or less, about 10 mg or less, or about 5 mg or less of the dry powder, e.g., about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, or about 20 mg, of the dry powder.

The dry particles can include any pharmaceutically acceptable salt of DHE, such as a methanesulfonic acid salt (mesylate salt), tartrate salt, or sulfate salt of DHE. In preferred aspects, the dry powder comprises DHE mesylate. In some aspects, the dry powder comprises DHE or a salt thereof in the monohydrate form, the anhydrous form, or a combination of both.

The DHE or a salt or hydrate thereof can be present in the dry particles in substantially amorphous form, in substantially crystalline form, or as a mixture of both forms, e.g., as determined by X-ray powder diffraction (XRPD).

In a preferred aspect, the DHE or a salt, hydrate, or polymorph thereof is substantially amorphous. For example, the DHE or a salt, hydrate, or polymorph thereof can be less than about 5% crystalline, less than about 4% crystalline, less than about 3% crystalline, less than about 2% crystalline, or less than about 1% crystalline, as determined by XRPD. In some embodiments, amorphous DHE mesylate is provided by dissolving DHE mesylate in a suitable liquid, which removes crystallinity, and including the DHE solution in the feedstock with other components for spray drying to produce dry powders that contain substantially amorphous DHE. The state of the DHE in the dry powder can be further confirmed by XRPD of the dry powder.

In another aspect, the DHE or a salt, hydrate, or polymorph thereof is at least about 50% crystalline (e.g., at least about 60%, at least about 70%, at least about 80%, at least about 90%, at least about 95%, or about 100% crystalline) as determined by XRPD. In another aspect, the DHE or a salt or hydrate thereof is substantially crystalline, e.g., at least about 95% crystalline, at least about 96% crystalline, at least about 97% crystalline, at least about 98% crystalline, at least about 99% crystalline, or 100% crystalline, as determined by XRPD. In some embodiments, the crystallinity of DHE mesylate is confirmed by XRPD prior to spray drying, and then spray dried as a stabilized suspension (e.g., containing polysorbate 80) of crystalline DHE mesylate particles of the desired size, to ensure the resulting dry powder contains crystalline DHE mesylate, which can be further confirmed by XRPD of the dry powder.

The DHE or a salt, hydrate, or polymorph thereof may be present in the respirable dry particles in crystalline particulate form (e.g., microcrystalline or nanocrystalline form). For example, the crystalline DHE (e.g., DHE mesylate) can be in the form of a sub-particle of about 50 nm to about 5000 nm (Dv50). In some embodiments, the sub-particle size is about 100 nm, about 300 nm, about 1000 nm, about 1500 nm, about 80 nm to about 300 nm, about 80 nm to about 250 nm, about 80 nm to about 200 nm, about 100 nm to about 150 nm, about 1200 nm to about 1500 nm, about 1500 nm to about 1750 nm, about 1200 nm to about 1400 nm, or about 1200 nm to about 1350 nm (Dv50). In particular embodiments, the sub-particle is between about 50 nm to about 2500 nm, between about 50 nm and 1000 nm, between about 50 nm and 800 nm, between about 50 nm and 600 nm, between about 50 nm and 500 nm, between about 50 nm and 400 nm, between about 50 nm and 300 nm, between about 50 nm and 200 nm, or between about 100 nm and 300 nm (Dv50). In one embodiment, the sub-particle is about 50 to about 200 nm (Dv50). In one embodiment, the sub-particle is about 100 nm (Dv50). In one embodiment, the sub-particle is about 900 nm to about 1100 nm (Dv50). In one embodiment, the sub-particle is about 1000 nm. In an embodiment, the DHE or a salt, hydrate, or polymorph thereof is present in the respirable dry particles in microcrystalline form. In an embodiment, the DHE or a salt, hydrate, or polymorph thereof is present in the respirable dry particles in nanocrystalline form.

The DHE or a salt, polymorph, or hydrate thereof can be prepared in any desired sub-particle size using a suitable method, including a stabilizer if desired, such as by wet milling, jet milling, or another suitable method. In some aspects, the DHE or a salt thereof (e.g., DHE mesylate) in crystalline particulate form is prepared by milling. In some embodiments, the milling comprises jar roller milling, Netzsch MicroCer Batch Mode milling, Netzsch MiniCer Recirculation Mode milling, jet milling (e.g., using a Sturtevant Qualification Micronizer), or a combination thereof. In preferred aspects, crystalline forms of the DHE or salt thereof are milled.

Without wishing to be bound by theory, it is believed that amorphous DHE (e.g., DHE mesylate) dissolves in the airway lining fluid more rapidly than crystalline DHE, which can result in desired pharmacokinetic properties (e.g., rapid Tₘₐₓ, lower AUC and shorter t_{1/2}), to provide maximal efficacy in the shortest time possible, while avoiding unnecessary drug exposure and reducing potential side effects.

### Metal Cation Salts, Excipients, and Stabilizers

The respirable dry particles described herein typically contain a monovalent metal cation salt, one or more excipients, and optionally further contain a stabilizer or other additive.

The respirable dry particles can contain between 1% to 85% of a monovalent metal cation salt by weight (wt %). For example, the respirable dry particles can contain a monovalent metal cation salt in an amount of between about 5% to about 15%, between about 15% to about 25%, between about 25% to about 35%, between about 35% to about 45%, between about 45% to about 55%, between about 55% to about 65%, between about 65% to about 75%, or between about 75% to about 85% by weight. In a preferred aspect, the respirable dry particles contain about 5% to about 15% by weight, e.g., about 5%, 6%, 7%, 8%, 9%, 10%, 11%, 12%, 13%, 14%, or 15% monovalent metal cation salt by weight. In another preferred aspect, the respirable dry particles contain about 40% to about 50% by weight, e.g., about 41%, 42%, 43%, 44%, 45%, 46%, 47%, 48%, 49%, or 50% monovalent metal cation salt by weight. In another preferred aspect, the respirable dry particles contain about 70% to about 80% by weight, e.g., about 71%, 2%, 73%, 74%, 75%, 76%, 77%, 78%, 79%, or 80% monovalent metal cation salt by weight. In an embodiment, the respirable dry particles contain 9.0% monovalent metal cation salt by weight. In an embodiment, the respirable dry particles contain 9.7% monovalent metal cation salt by weight.

Preferred monovalent metal cation salts (e.g., sodium salts, potassium salts) have one, or preferably two or more of the following characteristics: (i) can be processed into respirable dry powders, (ii) possess sufficient physicochemical stability in dry powder form to facilitate the production of a powder that is dispersible and physically stable over a range of conditions, including upon exposure to elevated humidity, (iii) undergo rapid dissolution upon deposition in the lungs, for example, half of the mass of the cation of the monovalent metal salt can be dissolved in less than 30 minutes, less than 15 minutes, less than 5 minutes, less than 2 minutes, less than 1 minute, or less than 30 seconds, and (iv) do not possess properties that can result in poor tolerability or adverse events, such as a significant exothermic or endothermic heat of solution (ΔH) for example, a ΔH lower than of about −10 kcal/mol or greater than about 10 kcal/mol. Rather, a preferred ΔH is between about −9 kcal/mol and about 9 kcal/mol, between about −8 kcal/mol and about 8 kcal/mol, between about −7 kcal/mol and about 7 kcal/mol, between about −6 kcal/mol and about 6 kcal/mol, between about −5 kcal/mol and about 5 kcal/mol, between about −4 kcal/mol and about 4 kcal/mol, between about −3 kcal/mol and about 3 kcal/mol, between about −2 kcal/mol and about 2 kcal/mol, between about −1 kcal/mol and about 1 kcal/mol, or about 0 kcal/mol.

Preferred monovalent metal cation salts include sodium, potassium and lithium salts, which can have desired solubility characteristics. In general, highly or moderately soluble sodium and potassium salts are preferred. For example, sodium and potassium salts that are contained in the respirable dry particles and dry powders can have a solubility in distilled water at room temperature (20-30° C.) and 1 bar of at least about 0.4 g/L, at least about 0.85 g/L, at least about 0.90 g/L, at least about 0.95 g/L, at least about 1.0 g/L, at least about 2.0 g/L, at least about 5.0 g/L, at least about 6.0 g/L, at least about 10.0 g/L, at least about 20 g/L, at least about 50 g/L, at least about 90 g/L, at least about 120 g/L, at least about 500 g/L, at least about 700 g/L or at least about 1000 g/L. Preferably, the sodium and potassium salts have a solubility greater than about 0.90 g/L, greater than about 2.0 g/L, or greater than about 90 g/L. Alternatively, the sodium and potassium salts that are contained in the respirable dry particles and dry powders can have a solubility in distilled water at room temperature (20-30° C.) and 1 bar of between at least about 0.4 g/L to about 200 g/L, between about 1.0 g/L to about 120 g/L, between 5.0 g/L to about 50 g/L.

Suitable sodium salts that can be present in the respirable dry particles of the invention include, for example, sodium chloride, sodium citrate, sodium sulfate, sodium lactate, sodium acetate, sodium bicarbonate, sodium carbonate, sodium stearate, sodium ascorbate, sodium benzoate, sodium biphosphate, dibasic sodium phosphate, sodium phosphate, sodium bisulfite, sodium borate, sodium gluconate, sodium metasilicate, sodium propionate, sodium methanesulfonate, and the like. In a preferred aspect, the dry powders and dry particles comprise sodium chloride. In another preferred aspect, the dry powders and dry particles comprise sodium sulfate.

Suitable potassium salts include, for example, potassium chloride, potassium citrate, potassium bromide, potassium iodide, potassium bicarbonate, potassium nitrite, potassium persulfate, potassium sulfite, potassium sulfate, potassium bisulfite, potassium phosphate, potassium acetate, potassium citrate, potassium glutamate, dipotassium guanylate, potassium gluconate, potassium malate, potassium ascorbate, potassium sorbate, potassium succinate, potassium sodium tartrate and any combination thereof. For example, the dry powders and dry particles include potassium chloride, potassium citrate, potassium phosphate, potassium sulfate, or any combination of these salts.

Suitable lithium salts include, for example, lithium chloride, lithium bromide, lithium carbonate, lithium nitrate, lithium sulfate, lithium acetate, lithium lactate, lithium citrate, lithium aspartate, lithium gluconate, lithium malate, lithium ascorbate, lithium orotate, lithium succinate or any combination of these salts.

Dry powder and particles of the invention can contain a high percentage of sodium salt and/or potassium salt in the composition, and can be sodium cation (Na⁺) and/or potassium cation (K⁺) dense. The dry particles may contain 3% or more, 5% or more, 10% or more, 15% or more, 20% or more, 25% or more, 30% or more, 35% or more, 40% or more, 50% or more, 60% or more, 70% or more, 75% or more, 80% or more, 85% or more, 90% or more, or 95% or more sodium salt or potassium salt by weight. In a preferred aspect, the dry powders and dry particles contain between about 4% and about 14% of sodium salt, such as sodium chloride (e.g., between about 6% and about 12%, or about 5%, about 6%, about 7%, about 8%, about 9%, about 10%, about 11%, about 12%, about 13%, about 14%, or about 15%) by weight. In another preferred aspect, the dry powders and dry particles contain between about 40% and about 50% of sodium salt, such as sodium chloride (e.g., between about 43% and about 48%, or about 40%, about 41%, about 42%, about 43%, about 44%, about 45%, about 46%, about 47%, about 48%, about 49%, or about 50%) by weight. In yet another preferred aspect, the dry powders and dry particles contain between about 70% and about 80% of sodium salt by weight (e.g., between about 74% and about 78%, or about 70%, about 71%, about 72%, about 73%, about 74%, about 75%, about 76%, about 77%, about 78%, about 79%, or about 80%).

Alternatively or in addition, the respirable dry powders and particles of the invention can contain a monovalent metal cation salt (e.g., sodium salt or potassium salt), which provides monovalent cation (e.g., Na⁺ or K⁺) in an amount of at least about 3% by weight of the respirable dry particles. For example, the respirable dry particles of the invention can include a sodium salt or potassium salt which provides Na⁺ or K⁺, in an amount of at least about 5% by weight, at least about 7% by weight, at least about 10% by weight, at least about 11% by weight, at least about 12% by weight, at least about 13% by weight, at least about 14% by weight, at least about 15% by weight, at least about 17% by weight, at least about 20% by weight, at least about 25% by weight, at least about 30% by weight, at least about 35% by weight, at least about 40% by weight, at least about 45% by weight, at least about 50% by weight, at least about 55% by weight, at least about 60% by weight, at least about 65% by weight or at least about 70% by weight of the respirable dry particles.

The respirable dry particles can be dense in monovalent metal cation salt (e.g. sodium salt or potassium salt), or can have low loading of monovalent metal cation salt. High salt content in dry powders is thought to be important for certain desirable characteristics of dry powders, such as high dispersibility and flow rate independence. Surprisingly, it was discovered that DHE-containing dry powders that included a low salt content had desirable dispersity and aerodynamic characteristics that were similar to those seen in preparations that contain much higher salt loads. For example, as illustrated in Example 1, Formulations I, XII, and XV which contain 76 wt %, 69 wt %, and 63 wt % sodium chloride, respectively, had had similar dispersibility ratios (1 bar/4 bar) and MMAD to Formulations V, XIII, and XVI which contain 9 wt %, 9.9 wt %, and 9 wt % sodium chloride, respectively (Tables 3 and 4). In some patients, dry powders that contain a high salt load can cause temporary cough or mucosal irritation. Beneficially, these can be reduced or eliminated with the low salt DHE formulations disclosed herein (e.g. Formulations V, XIII, and XVI).

If desired, the respirable dry powders and particles of the invention can contain one or more other salts in addition to the monovalent metal cation salt (e.g., sodium salt and/or potassium salt), such as one or more non-toxic salts of the elements magnesium, calcium, aluminum, silicon, scandium, titanium, vanadium, chromium, cobalt, nickel, copper, manganese, zinc, tin, silver and the like.

Suitable magnesium salts that can be present in the respirable dry particles described herein include, for example, magnesium fluoride, magnesium chloride, magnesium bromide, magnesium iodide, magnesium phosphate, magnesium sulfate, magnesium sulfite, magnesium carbonate, magnesium oxide, magnesium nitrate, magnesium borate, magnesium acetate, magnesium citrate, magnesium gluconate, magnesium maleate, magnesium succinate, magnesium malate, magnesium taurate, magnesium orotate, magnesium glycinate, magnesium naphthenate, magnesium acetylacetonate, magnesium formate, magnesium hydroxide, magnesium stearate, magnesium hexafluorosilicate, magnesium salicylate or any combination thereof. In some embodiments, the dry particles do not contain a magnesium salt.

Suitable calcium salts that can be present in the respirable dry particles described herein include, for example, calcium chloride, calcium sulfate, calcium lactate, calcium citrate, calcium carbonate, calcium acetate, calcium phosphate, calcium alginate, calcium stearate, calcium sorbate, calcium gluconate and the like. In some embodiments, the dry particles do not contain a calcium salt.

The respirable dry particles described herein can include an excipient (e.g., a physiologically or pharmaceutically acceptable excipient). The respirable dry particles may contain between about 1% and about 99% of one or more excipients by weight (wt %). For example, the dry particles may contain between about 10% and about 20%, between about 20% and about 30%, between about 30% and about 40%, between about 40% and about 50%, between about 50% and about 60%, between about 60% and about 70%, between about 70% and about 80%, or between about 80% and about 90% by weight excipient. In one aspect, the dry particles comprise one excipient. In another aspect, the dry particles comprise two excipients.

The one or more excipient can be a carbohydrate, sugar, sugar alcohol, oligosaccharide (e.g., a short oligosaccharide), or amino acid, either alone or in any desired combination. Preferred excipients are generally relatively free-flowing particulates that do not thicken or polymerize upon contact with water, and are toxicologically innocuous when inhaled as a dispersed powder. Carbohydrate excipients that are useful in this regard include the mono- and polysaccharides. Representative monosaccharides include carbohydrate excipients such as dextrose (anhydrous and the monohydrate; also referred to as glucose and glucose monohydrate), galactose, D-mannose, sorbose and the like. Representative disaccharides include lactose, maltose, sucrose, trehalose and the like. Representative trisaccharides include raffinose and the like. Other carbohydrate excipients include maltodextrin and cyclodextrins, such as 2-hydroxypropyl-beta-cyclodextrin can be used as desired. Representative sugar alcohols include mannitol, sorbitol and the like.

Suitable amino acid excipients include any of the naturally occurring amino acids that form a powder under standard pharmaceutical processing techniques and include the non-polar (hydrophobic) amino acids and polar (uncharged, positively charged and negatively charged) amino acids, such amino acids are of pharmaceutical grade and are generally regarded as safe (GRAS) by the U.S. Food and Drug Administration. Representative examples of non-polar amino acids include alanine, isoleucine, leucine, methionine, phenylalanine, proline, tryptophan and valine. Representative examples of polar, uncharged amino acids include cysteine, glycine, glutamine, serine, threonine, and tyrosine. Representative examples of polar, positively charged amino acids include arginine, histidine and lysine. Representative examples of negatively charged amino acids include aspartic acid and glutamic acid. These amino acids can be in the D or L optical isomer form, or a mixture of the two forms. These amino acids are generally available from commercial sources that provide pharmaceutical-grade products such as the Aldrich Chemical Company, Inc., Milwaukee, Wis. or Sigma Chemical Company, St. Louis, Mo.

Preferred amino acid excipients, such as the hydrophobic amino acid leucine, in the D or L optical form, or a mixture of the two forms, and can be present in the dry particles of the invention in an amount of about 99% or less by weight of respirable dry particles (wt %). For example, the respirable dry particles of the invention can contain the amino acid leucine in an amount of about 0.1% to about 10% by weight, 5% to about 30% by weight, about 10% to about 20% by weight, about 5% to about 20% by weight, about 11% to about 50% by weight, about 15% to about 50% by weight, about 20% to about 50% by weight, about 30% to about 50% by weight, about 11% to about 40% by weight, about 11% to about 30% by weight, about 11% to about 20% by weight, about 20% to about 40% by weight, about 51% to about 99% by weight, about 60% to about 99% by weight, about 70% to about 99% by weight, about 80% to about 99% by weight, about 51% to about 90% by weight, about 51% to about 80% by weight, about 51% to about 70% by weight, about 60% to about 90% by weight, about 70% to about 90% by weight, about 45% or less by weight, about 40% or less by weight, about 35% or less by weight, about 30% or less by weight, about 25% or less by weight, about 20% or less by weight, about 18% or less by weight, about 16% or less by weight, about 15% or less by weight, about 14% or less by weight, about 13% or less by weight, about 12% or less by weight, about 11% or less by weight, about 10% or less by weight, about 9% or less by weight, about 8% or less by weight, about 7% or less by weight, about 6% or less by weight, about 5% or less by weight, about 4% or less by weight, about 3% or less by weight, about 2% or less by weight, or about 1% or less by weight. In some preferred aspects, the respirable dry particles contain between about 15% and about 25% leucine by weight (e.g., about 15%, about 16%, about 17%, about 18%, about 19%, about 20%, about 21%, about 22%, about 23%, about 24%, or about 25% leucine by weight).

Preferred carbohydrate excipients, such as mannitol, can be present in the dry particles of the invention in an amount of about 99% or less by weight of respirable dry particles. For example, the respirable dry particles of the invention can contain mannitol in an amount of about 0.1% to about 10% by weight, 5% to about 30% by weight by weight, about 10% to about 20% by weight by weight, about 5% to about 20% by weight, about 11% to about 50% by weight, about 15% to about 50% by weight, about 20% to about 50% by weight, about 30% to about 50% by weight, about 11% to about 40% by weight, about 11% to about 30% by weight, about 11% to about 20% by weight, about 20% to about 40% by weight, about 51% to about 99% by weight, about 60% to about 99% by weight, about 70% to about 99% by weight, about 80% to about 99% by weight, about 51% to about 90% by weight, about 51% to about 80% by weight, about 51% to about 70% by weight, about 60% to about 90% by weight, about 70% to about 90% by weight, about 90% or less by weight, about 80% or less by weight, about 70% or less by weight, about 60% or less by weight, about 50% or less by weight, about 45% or less by weight, about 40% or less by weight, about 35% or less by weight, about 30% or less by weight, about 25% or less by weight, about 20% or less by weight, about 18% or less by weight, about 16% or less by weight, about 15% or less by weight, about 14% or less by weight, about 13% or less by weight, about 12% or less by weight, about 11% or less by weight, about 10% or less by weight, about 9% or less by weight, about 8% or less by weight, about 7% or less by weight, about 6% or less by weight, about 5% or less by weight, about 4% or less by weight, about 3% or less by weight, about 2% or less by weight, or about 1% or less by weight. In some preferred aspects, the respirable dry particles contain between about 55% to about 65% mannitol by weight (e.g., about 55%, about 56%, about 57%, about 58%, about 59%, about 60%, about 61%, about 62%, about 63%, about 64%, or about 65% mannitol by weight). In other preferred aspects, the respirable dry particles contain between about 40% to about 50% mannitol by weight (e.g., about 40%, about 41%, about 42%, about 43%, about 44%, about 45%, about 46%, about 47%, about 48%, about 49%, or about 50% mannitol by weight).

In some preferred aspects, the dry particles contain an excipient selected from leucine (e.g., L-leucine), maltodextrin, mannitol and any combination thereof. In particular embodiments, the dry particles contain leucine and mannitol.

The respirable dry particles of the invention can contain one or more stabilizers. In some aspects, the amount of stabilizer is less than about 10% by weight, or more preferably less than 5% by weight (e.g., about 4%, about 3%, about 2%, about 1%, or less by weight). Stabilizers that may be used in the dry particles include polysorbate 80 (PS80) and oleic acid, or salts thereof. In particular embodiments, the stabilizer is polysorbate 80 (PS80). Stabilizers are particularly useful in the manufacture of dry powders that contain crystalline DHE.

In one aspect, the respirable dry particles comprise DHE mesylate, sodium chloride, leucine (e.g., L-leucine), and mannitol, wherein the DHE mesylate is present in an amount of about 1% to about 25%; the sodium chloride is present in an amount of about 4% to about 14%; the mannitol is present in an amount of about 55% to about 75%; and the leucine is present in an amount of about 12% to about 25%; wherein all percentages are weight percentages on a dry basis and all the components of the respirable dry particles amount to 100%. The DHE mesylate can be amorphous (i.e., less than about 5% crystallinity), or crystalline (i.e., at least about 95% crystallinity). The crystalline DHE mesylate can be nano-sized or micro-sized, e.g., depending on the milling technique applied to the crystalline DHE mesylate.

In some preferred aspects, the respirable dry particles comprise between about 5 wt % to about 25 wt % DHE mesylate; between about 4 wt % to about 14 wt % sodium chloride; between about 13 wt % to about 23 wt % leucine (e.g., L-leucine); and between about 58 wt % to about 68 wt % mannitol. For example, the respirable dry particles can comprise between about 8 wt % to about 12 wt % DHE mesylate; between about 7 wt % to about 11 wt % sodium chloride; between about 16 wt % to about 20 wt % leucine (e.g., L-leucine); and between about 61 wt % to about 65 wt % mannitol. In an embodiment, the respirable dry particles comprise between about 9 wt % to about 11 wt % DHE mesylate; between about 8 wt % to about 10 wt % sodium chloride; between about 17 wt % to about 19 wt % leucine (e.g., L-leucine); and between about 62 wt % to about 64 wt % mannitol. In an embodiment, the respirable dry particles comprise about 10 wt % DHE mesylate; about 9 wt % sodium chloride; about 18 wt % leucine (e.g., L-leucine); and about 63 wt % mannitol.

In some embodiments, the respirable dry particles comprise between about 5 wt % to about 25 wt % DHE mesylate (amorphous); between about 4 wt % to about 14 wt % sodium chloride; between about 13 wt % to about 23 wt % leucine (e.g., L-leucine); and between about 58 wt % to about 68 wt % mannitol. For example, the respirable dry particles can comprise between about 8 wt % to about 12 wt % DHE mesylate (amorphous); between about 7 wt % to about 11 wt % sodium chloride; between about 16 wt % to about 20 wt % leucine (e.g., L-leucine); and between about 61 wt % to about 65 wt % mannitol. In an embodiment, the respirable dry particles comprise between about 9 wt % to about 11 wt % DHE mesylate (amorphous); between about 8 wt % to about 10 wt % sodium chloride; between about 17 wt % to about 19 wt % leucine (e.g., L-leucine); and between about 62 wt % to about 64 wt % mannitol. In an embodiment, the respirable dry particles comprise about 10 wt % DHE mesylate (amorphous); about 9 wt % sodium chloride; about 18 wt % leucine (e.g., L-leucine); and about 63 wt % mannitol.

In some embodiments, the respirable dry particles comprise between about 5 wt % to about 25 wt % DHE mesylate (crystalline); between about 4 wt % to about 14 wt % sodium chloride; between about 13 wt % to about 23 wt % leucine (e.g., L-leucine); and between about 58 wt % to about 68 wt % mannitol. For example, the respirable dry particles can comprise between about 8 wt % to about 12 wt % DHE mesylate (crystalline); between about 7 wt % to about 11 wt % sodium chloride; between about 16 wt % to about 20 wt % leucine (e.g., L-leucine); and between about 61 wt % to about 65 wt % mannitol. In an embodiment, the respirable dry particles comprise between about 9 wt % to about 11 wt % DHE mesylate (crystalline); between about 8 wt % to about 10 wt % sodium chloride; between about 17 wt % to about 19 wt % leucine (e.g., L-leucine); and between about 62 wt % to about 64 wt % mannitol. In an embodiment, the respirable dry particles comprise about 10 wt % DHE mesylate (crystalline); about 9 wt % sodium chloride; about 18 wt % leucine (e.g., L-leucine); and about 63 wt % mannitol.

In other preferred aspects, the respirable dry particles comprise between about 0.1 wt % to about 8 wt % DHE mesylate; between about 4 wt % to about 15 wt % sodium chloride; between about 13 wt % to about 25 wt % leucine (e.g., L-leucine); and between about 62 wt % to about 73 wt % mannitol. For example, the respirable dry particles can comprise between about 1 wt % to about 5 wt % DHE mesylate; between about 8 wt % to about 12 wt % sodium chloride; between about 17 wt % to about 22 wt % leucine (e.g., L-leucine); and between about 66 wt % to about 70 wt % mannitol. In an embodiment, the respirable dry particles comprise between about 2 wt % to about 4 wt % DHE mesylate; between about 8.7 wt % to about 10.7 wt % sodium chloride; between about 18.4 wt % to about 20.4 wt % leucine (e.g., L-leucine); and between about 66.9 wt % to about 68.9 wt % mannitol. In an embodiment, the respirable dry particles comprise about 3 wt % DHE mesylate; about 9.7 wt % sodium chloride; about 19.4 wt % leucine (e.g., L-leucine); and about 67.9 wt % mannitol.

In some embodiments, the respirable dry particles comprise between about 0.1 wt % to about 8 wt % DHE mesylate (amorphous); between about 4 wt % to about 15 wt % sodium chloride; between about 13 wt % to about 25 wt % leucine (e.g., L-leucine); and between about 62 wt % to about 73 wt % mannitol. For example, the respirable dry particles can comprise between about 1 wt % to about 5 wt % DHE mesylate (amorphous); between about 8 wt % to about 12 wt % sodium chloride; between about 17 wt % to about 22 wt % leucine (e.g., L-leucine); and between about 66 wt % to about 70 wt % mannitol. In an embodiment, the respirable dry particles comprise between about 2 wt % to about 4 wt % DHE mesylate (amorphous); between about 8.7 wt % to about 10.7 wt % sodium chloride; between about 18.4 wt % to about 20.4 wt % leucine (e.g., L-leucine); and between about 66.9 wt % to about 68.9 wt % mannitol. In an embodiment, the respirable dry particles comprise about 3 wt % DHE mesylate (amorphous); about 9.7 wt % sodium chloride; about 19.4 wt % leucine (e.g., L-leucine); and about 67.9 wt % mannitol.

In some embodiments, the respirable dry particles comprise between about 0.1 wt % to about 8 wt % DHE mesylate (crystalline); between about 4 wt % to about 15 wt % sodium chloride; between about 13 wt % to about 25 wt % leucine (e.g., L-leucine); and between about 62 wt % to about 73 wt % mannitol. For example, the respirable dry particles can comprise between about 1 wt % to about 5 wt % DHE mesylate (crystalline); between about 8 wt % to about 12 wt % sodium chloride; between about 17 wt % to about 22 wt % leucine (e.g., L-leucine); and between about 66 wt % to about 70 wt % mannitol. In an embodiment, the respirable dry particles comprise between about 2 wt % to about 4 wt % DHE mesylate (crystalline); between about 8.7 wt % to about 10.7 wt % sodium chloride; between about 18.4 wt % to about 20.4 wt % leucine (e.g., L-leucine); and between about 66.9 wt % to about 68.9 wt % mannitol. In an embodiment, the respirable dry particles comprise about 3 wt % DHE mesylate (crystalline); about 9.7 wt % sodium chloride; about 19.4 wt % leucine (e.g., L-leucine); and about 67.9 wt % mannitol.

In another aspect, the respirable dry particles comprise DHE mesylate, sodium chloride, and leucine (e.g., L-leucine), wherein the DHE mesylate is present in an amount of about 0.1% to about 15%; the sodium chloride is present in an amount of about 65% to about 85%; and the leucine is present in an amount of about 12% to about 25%; wherein all percentages are weight percentages on a dry basis and all the components of the respirable dry particles amount to 100%. For example, the respirable dry particles can comprise between about 1 wt % to about 10 wt % DHE mesylate; between about 71 wt % to about 81 wt % sodium chloride; and between about 14 wt % to about 24 wt % leucine (e.g., L-leucine). In an embodiment, the respirable dry particles comprise between about 1 wt % to about 10 wt % DHE mesylate (amorphous); between about 71 wt % to about 81 wt % sodium chloride; and between about 14 wt % to about 24 wt % leucine (e.g., L-leucine). In an embodiment, the respirable dry particles comprise between about 4 wt % to about 6 wt % DHE mesylate; between about 75 wt % to about 77 wt % sodium chloride; and between about 18 wt % to about 20 wt % leucine (e.g., L-leucine). In an embodiment, the respirable dry particles comprise between about 4 wt % to about 6 wt % DHE mesylate (amorphous); between about 75 wt % to about 77 wt % sodium chloride; and between about 18 wt % to about 20 wt % leucine (e.g., L-leucine). In an embodiment, the respirable dry particles comprise about 5 wt % DHE mesylate; about 76 wt % sodium chloride; and about 19 wt % leucine (e.g., L-leucine). In an embodiment, the respirable dry particles comprise about 5 wt % DHE mesylate (amorphous); about 76 wt % sodium chloride; and about 19 wt % leucine (e.g., L-leucine).

In another aspect, the respirable dry particles comprise DHE mesylate, sodium chloride, and mannitol, wherein the DHE mesylate is present in an amount of about 0.1% to about 15%; the sodium chloride is present in an amount of about 38% to about 58%; and the mannitol is present in an amount of about 38% to about 58%; wherein all percentages are weight percentages on a dry basis and all the components of the respirable dry particles amount to 100%. For example, the respirable dry particles can comprise between about 1 wt % to about 10 wt % DHE mesylate; between about 43 wt % to about 52 wt % sodium chloride; and between about 43 wt % to about 52 wt % mannitol. In an embodiment, the respirable dry particles comprise between about 1 wt % to about 10 wt % DHE mesylate (amorphous); between about 43 wt % to about 52 wt % sodium chloride; and between about 43 wt % to about 52 wt % mannitol. In an embodiment, the respirable dry particles comprise between about 4 wt % to about 6 wt % DHE mesylate; between about 46.5 wt % to about 48.5 wt % sodium chloride; and between about 46.5 wt % to about 48.5 wt % mannitol. In an embodiment, the respirable dry particles comprise between about 4 wt % to about 6 wt % DHE mesylate (amorphous); between about 46.5 wt % to about 48.5 wt % sodium chloride; and between about 46.5 wt % to about 48.5 wt % mannitol. In an embodiment, the respirable dry particles comprise about 5 wt % DHE mesylate; about 47.5 wt % sodium chloride; and about 47.5 wt % mannitol. In an embodiment, the respirable dry particles comprise about 5 wt % DHE mesylate (amorphous); about 47.5 wt % sodium chloride; and about 47. wt % mannitol.

In another aspect, the respirable dry particles comprise DHE mesylate, sodium sulfate, mannitol, and polysorbate 80, wherein the DHE mesylate is present in an amount of about 1% to about 25%; the sodium sulfate is present in an amount of about 38% to about 58%; the mannitol is present in an amount of about 38% to about 58%; and the polysorbate 80 is present in an amount of about 0.1% to about 5%; wherein all percentages are weight percentages on a dry basis and all the components of the respirable dry particles amount to 100%. For example, the respirable dry particles can comprise between about 5 wt % to about 15 wt % DHE mesylate; between about 43 wt % to about 52 wt % sodium sulfate; between about 43 wt % to about 52 wt % mannitol; and between about 0.2% to about 2.0% polysorbate 80. In an embodiment, the respirable dry particles comprise between about 5 wt % to about 15 wt % DHE mesylate (crystalline, nano-sized); between about 43 wt % to about 52 wt % sodium sulfate; between about 43 wt % to about 52 wt % mannitol; and between about 0.2% to about 2.0% polysorbate 80. In an embodiment, the respirable dry particles comprise between about 5 wt % to about 15 wt % DHE mesylate (crystalline, micro-sized); between about 43 wt % to about 52 wt % sodium sulfate; between about 43 wt % to about 52 wt % mannitol; and between about 0.2% to about 2.0% polysorbate 80. In an embodiment, the respirable dry particles comprise between about 9 wt % to about 11 wt % DHE mesylate; between about 46.5 wt % to about 48.5 wt % sodium sulfate; between about 46.5 wt % to about 48.5 wt % mannitol; and between about 0.5 wt % and 1.5 wt % polysorbate 80. In an embodiment, the respirable dry particles comprise between about 9 wt % to about 11 wt % DHE mesylate (crystalline, nano-sized); between about 46.5 wt % to about 48.5 wt % sodium sulfate; between about 46.5 wt % to about 48.5 wt % mannitol; and between about 0.5 wt % and 1.5 wt % polysorbate 80. In an embodiment, the respirable dry particles comprise between about 9 wt % to about 11 wt % DHE mesylate (crystalline, micro-sized); between about 46.5 wt % to about 48.5 wt % sodium sulfate; between about 46.5 wt % to about 48.5 wt % mannitol; and between about 0.5 wt % and 1.5 wt % polysorbate 80. In an embodiment, the respirable dry particles comprise about 10 wt % DHE mesylate; about 47.5 wt % sodium sulfate; about 47 wt % mannitol; about 1 wt % polysorbate 80. In an embodiment, the respirable dry particles comprise about 10 wt % DHE mesylate (crystalline, nano-sized); about 47.5 wt % sodium sulfate; about 47 wt % mannitol; about 1 wt % polysorbate 80. In an embodiment, the respirable dry particles comprise about 10 wt % DHE mesylate (crystalline, micro-sized); about 47.5 wt % sodium sulfate; about 47 wt % mannitol; about 1 wt % polysorbate 80.

In another aspect, the respirable dry particles comprise DHE mesylate, sodium chloride, mannitol, and leucine (e.g., L-leucine), wherein the DHE mesylate is present in an amount of between about 10% and about 40%; the sodium chloride is present in an amount of between about 1% and about 20%; the mannitol is present in an amount of between about 40% and about 80%; and the leucine is present in an amount of between about 10% and about 30%, wherein all percentages are weight percentages on a dry basis and all the components of the respirable dry particles amount to 100%. For example, the respirable dry particles can comprise between about 12 wt % to about 35 wt % DHE mesylate; between about 3 wt % to about 15 wt % sodium chloride; between about 45 wt % to about 75 wt % mannitol; and between about 12 wt % and about 24 wt % leucine. In a particular aspect, the respirable dry particles comprise between about 12 wt % to about 35 wt % DHE mesylate (amorphous); between about 3 wt % to about 15 wt % sodium chloride; between about 45 wt % to about 75 wt % mannitol; and between about 12 wt % and about 24 wt % leucine. In a more particular aspect, the respirable dry particles comprise between about 14 wt % to about 31 wt % DHE mesylate (amorphous); between about 4 wt % to about 10 wt % sodium chloride; between about 42 wt % to about 72 wt % mannitol; and between about 17 wt % and about 19 wt % leucine.

Particular dry powders and respirable dry particles have the following formulations shown in Table 1.

TABLE 1
Exemplary Dry Powder Formulations

| | *API | Monovalent Salt | Excipient(s) |
|---|---|---|---|
| Formulation | (wt %) | (wt %) | (wt %) |
| I | DHE mesylate | Sodium chloride | L-Leucine, 19.0% |
| | 5.0% | 76.0% | |
| II | DHE mesylate | Sodium chloride | Mannitol, 47.5% |
| | 5.0% | 47.5% | |
| III | DHE mesylate | Sodium sulfate | Mannitol, 44.5% |
| | 10.0% | 44.5% | Polysorbate 80, 1.0% |
| IV | DHE mesylate | Sodium sulfate | Mannitol, 44.5% |
| | 10.0% | 44.5% | Polysorbate 80, 1.0% |
| V | DHE mesylate | Sodium chloride | Mannitol, 63.0% |
| | 10.0% | 9.0% | L-Leucine, 18.0% |
| VI | DHE mesylate | Sodium chloride | L-Leucine, 19.7% |
| | 1.5% | 78.8% | |
| VII | DHE mesylate | Sodium chloride | Mannitol, 49.25% |
| | 1.5% | 49.25% | |
| VIII | DHE mesylate | Sodium chloride | L-Leucine, 18.0% |
| | 10.0% | 72.0% | |
| IX | DHE mesylate | Sodium chloride | Mannitol, 45.0% |
| | 10.0% | 45.0% | |
| X | DHE mesylate | Sodium chloride | Mannitol, 39.4% |
| | 1.5% | 39.4% | L-Leucine, 19.7% |
| XI | DHE mesylate | Sodium chloride | Mannitol, 36.0% |
| | 10.0% | 36.0% | L-Leucine, 18.0% |
| XII | DHE mesylate | Sodium chloride | Mannitol, 9.85% |
| | 1.5% | 68.95% | L-Leucine, 19.7% |
| XIII | DHE mesylate | Sodium chloride | Mannitol, 68.95% |
| | 1.5% | 9.85% | L-Leucine, 19.7% |
| XIV | DHE mesylate | Sodium chloride | Mannitol, 37.7% |
| | 5.75% | 37.7% | L-Leucine, 18.85% |
| XV | DHE mesylate | Sodium chloride | Mannitol, 9.0% |
| | 10.0% | 63.0% | L-Leucine, 18.0% |
| XVI | DHE mesylate | Sodium chloride | Mannitol, 63.0% |
| | 10.0% | 9.0% | L-Leucine, 18.0% |
| XVII | DHE mesylate | Sodium chloride | Mannitol, 58.0% |
| | 15.0% | 9.0% | L-Leucine, 18.0% |
| XVIII | DHE mesylate | Sodium chloride | Mannitol, 55.0% |
| | 18.0% | 9.0% | L-Leucine, 18.0% |
| XIX | DHE mesylate | Sodium chloride | Mannitol, 53.0% |
| | 20.0% | 9.0% | L-Leucine, 18.0% |
| XX | DHE mesylate | Sodium chloride | L-Leucine, 18.0% |
| | 20.0% | 62.0% | |
| XXI | DHE mesylate | Sodium chloride | Mannitol, 57.0% |
| | 20.0% | 5.0% | L-Leucine, 18.0% |
| XXII | DHE mesylate | Sodium chloride | Mannitol, 71.0% |
| | 20.0% | 9.0% | |
| XXIII | DHE mesylate | Sodium chloride | Mannitol, 51.0% |
| | 22.0% | 9.0% | L-Leucine, 18.0% |
| XXIV | DHE mesylate | Sodium chloride | Mannitol, 48.0% |
| | 25.0% | 9.0% | L-Leucine, 18.0% |
| XXV | DHE mesylate | Sodium chloride | Mannitol, 43.0% |
| | 30.0% | 9.0% | L-Leucine, 18.0% |

*Formulations I-II and V-XXV contain amorphous DHE mesylate; Formulation III contains nanocrystalline DHE mesylate; Formulation IV contains microcrystalline DHE mesylate.

### Dry Powder and Dry Particle Properties

The dry particles of the invention are preferably small, dense, and dispersible. Generally, the dry particles of the invention have a volume median geometric diameter (VMGD) as measured by HELOS/RODOS at 1.0 bar of about 10 μm or less (e.g., about 0.1 μm to about 10 μm). Preferably, the dry particles of the invention have a VMGD of about 9 μm or less (e.g., about 0.1 μm to about 9 μm), about 8 μm or less (e.g., about 0.1 μm to about 8 μm), about 7 μm or less (e.g., about 0.1 μm to about 7 μm), about 6 μm or less (e.g., about 0.1 μm to about 6 μm), about 5 μm or less (e.g., less than 5 μm, about 0.1 μm to about 5 μm), about 4 μm or less (e.g., 0.1 μm to about 4 μm), about 3 μm or less (e.g., 0.1 μm to about 3 μm), about 2 μm or less (e.g., 0.1 μm to about 2 μm), about 1 μm or less (e.g., 0.1 μm to about 1 μm), about 1 μm to about 6 μm, about 1 μm to about 5 μm, about 1 μm to about 4 μm, about 1 μm to about 3 μm, or about 1 μm to about 2 μm as measured by HELOS/RODOS at 1.0 bar.

The respirable dry powders can have a Hausner Ratio that is at least 1.5, and can be at least 1.6, at least 1.7, at least 1.8, at least 1.9, at least 2.0, at least 2.1, at least 2.2, at least 2.3, at least 2.4, at least 2.5, at least 2.6 or at least 2.7.

Generally, the dry particles of the invention are dispersible, and have 1 bar/4 bar and/or 0.5 bar/4 bar, and/or 0.2 bar/4 bar, and/or 0.2 bar/2 bar, of about 2.2 or less (e.g., about 1.0 to about 2.2) or about 2.0 or less (e.g., about 1.0 to about 2.0). Preferably, the dry particles of the invention have 1 bar/4 bar, and/or 0.5 bar/4 bar, of about 1.9 or less (e.g., about 1.0 to about 1.9), about 1.8 or less (e.g., about 1.0 to about 1.8), about 1.7 or less (e.g., about 1.0 to about 1.7), about 1.6 or less (e.g., about 1.0 to about 1.6), about 1.5 or less (e.g., about 1.0 to about 1.5), about 1.4 or less (e.g., about 1.0 to about 1.4), about 1.3 or less (e.g., less than 1.3, about 1.0 to about 1.3), about 1.2 or less (e.g., 1.0 to about 1.2), about 1.1 or less (e.g., 1.0 to about 1.1 μm) or the dry particles of the invention have 1 bar/4 bar and/or 0.5 bar/4 bar of about 1.0. Preferably 1 bar/4 bar and/or 0.5 bar/4 bar are measured by laser diffraction using a HELOS/RODOS system.

Alternatively, or in addition, the respirable dry particles of the invention can have an MMAD of about 10 microns or less, such as an MMAD of about 0.5 micron to about 10 microns. Preferably, the dry particles of the invention have an MMAD of about 5 microns or less (e.g., about 0.5 micron to about 5 microns, preferably about 1 micron to about 5 microns), about 4 microns or less (e.g., about 1 micron to about 4 microns), about 3.8 microns or less (e.g., about 1 micron to about 3.8 microns), about 3.5 microns or less (e.g., about 1 micron to about 3.5 microns), about 3.2 microns or less (e.g., about 1 micron to about 3.2 microns), about 3 microns or less (e.g., about 1 micron to about 3.0 microns), about 2.8 microns or less (e.g., about 1 micron to about 2.8 microns), about 2.2 microns or less (e.g., about 1 micron to about 2.2 microns), about 2.0 microns or less (e.g., about 1 micron to about 2.0 microns) or about 1.8 microns or less (e.g., about 1 micron to about 1.8 microns).

Alternatively, or in addition, the respirable dry powders and dry particles of the invention can have a fine particle fraction (FPF) of less than about 5.6 microns (FPF<5.6 μm) of at least about 20%, at least about 30%, at least about 40%, preferably at least about 45%, at least about 50%, at least about 55%, at least about 60%, at least about 65%, or at least about 70%.

Alternatively, or in addition, the dry powders and dry particles of the invention have a FPF of less than 5.0 microns (FPF_TD<5.0 μm) of at least about 20%, at least about 30%, at least about 45%, preferably at least about 40%, at least about 45%, at least about 50%, at least about 60%, at least about 65% or at least about 70%. Alternatively, or in addition, the dry powders and dry particles of the invention have a FPF of less than 5.0 microns of the emitted dose (FPF_ED<5.0 μm) of at least about 45%, preferably at least about 50%, at least about 60%, at least about 65%, at least about 70%, at least about 75%, at least about 80%, or at least about 85%. Alternatively, or in addition, the dry powders and dry particles of the invention can have an FPF of less than about 3.4 microns (FPF<3.4 μm) of at least about 20%, preferably at least about 25%, at least about 30%, at least about 35%, at least about 40%, at least about 45%, at least about 50%, or at least about 55%.

Alternatively, or in addition, the respirable dry powders and dry particles of the invention have a tap density of about 0.1 g/cm³ to about 1.0 g/cm³. For example, the small and dispersible dry particles have a tap density of about 0.1 g/cm³ to about 0.9 g/cm³, about 0.2 g/cm³ to about 0.9 g/cm³, about 0.2 g/cm³ to about 0.9 g/cm³, about 0.3 g/cm³ to about 0.9 g/cm³, about 0.4 g/cm³ to about 0.9 g/cm³, about 0.5 g/cm³ to about 0.9 g/cm³, or about 0.5 g/cm³ to about 0.8 g/cm³, greater than about 0.4 g/cc, greater than about 0.5 g/cc, greater than about 0.6 g/cc, greater than about 0.7 g/cc, about 0.1 g/cm³ to about 0.8 g/cm³, about 0.1 g/cm³ to about 0.7 g/cm³, about 0.1 g/cm³ to about 0.6 g/cm³, about 0.1 g/cm³ to about 0.5 g/cm³, about 0.1 g/cm³ to about 0.4 g/cm³, about 0.1 g/cm³ to about 0.3 g/cm³, or less than 0.3 g/cm³. In an embodiment, tap density is greater than about 0.4 g/cm³. In another embodiment, tap density is greater than about 0.5 g/cm³. Alternatively, tap density can be less than about 0.4 g/cc.

Alternatively, or in addition, the respirable dry powders and dry particles of the invention can have a water or solvent content of less than about 15% by weight of the respirable dry particle. For example, the respirable dry particles of the invention can have a water or solvent content of less than about 15% by weight, less than about 13% by weight, less than about 11.5% by weight, less than about 10% by weight, less than about 9% by weight, less than about 8% by weight, less than about 7% by weight, less than about 6% by weight, less than about 5% by weight, less than about 4% by weight, less than about 3% by weight, less than about 2% by weight, less than about 1% by weight, or be anhydrous. The respirable dry particles of the invention can have a water or solvent content of less than about 6% and greater than about 1%, less than about 5.5% and greater than about 1.5%, less than about 5% and greater than about 2%, about 2%, about 2.5%, about 3%, about 3.5%, about 4%, about 4.5% about 5%.

In addition to any of the features and properties described herein, in any combination, the respirable dry particles can have a heat of solution that is not highly exothermic. Preferably, the heat of solution is determined using the ionic liquid of a simulated lung fluid (e.g., as described in Moss, O. R. 1979. Simulants of lung interstitial fluid. Health Phys. 36, 447-448; or in Sun, G. 2001. Oxidative interactions of synthetic lung epithelial lining fluid with metal-containing particulate matter. Am. J. Physiol. Lung Cell. Mol. Physiol. 281, L807-L815) at pH 7.4 and 37° C. in an isothermal calorimeter. For example, the respirable dry particles can have a heat of solution that is less exothermic than the heat of solution of calcium chloride dihydrate, e.g., have a heat of solution that is greater than about −10 kcal/mol, greater than about −9 kcal/mol, greater than about −8 kcal/mol, greater than about −7 kcal/mol, greater than about −6 kcal/mol, greater than about −5 kcal/mol, greater than about −4 kcal/mol, greater than about −3 kcal/mol, greater than about −2 kcal/mol, greater than about −1 kcal/mol, or about −10 kcal/mol to about 10 kcal/mol.

The respirable dry powders and dry particles are characterized by a high emitted dose (e.g., CEPM of at least 40%, at least 45%, at least 50%, at least 55%, at least 60%, at least 65%, at least 70%, at least 75%, at least 80%, at least 85%, at least 90%, or at least 95%) from a dry powder inhaler when a total inhalation energy of less than about 2 Joules or less than about 1 Joule, or less than about 0.8 Joule, or less than about 0.5 Joule, or less than about 0.3 Joule is applied to the dry powder inhaler. The dry powder can fill the unit dose container, or the unit dose container can be at least 10% full, at least 20% full, at least 30% full, at least 40% full, at least 50% full, at least 60% full, at least 70% full, at least 80% full, or at least 90% full. The unit dose container can be a capsule (e.g., size 000, 00, 0E, 0, 1, 2, 3, and 4, with respective volumetric capacities of 1.37 mL, 950 μL, 770 μL, 680 μL, 480 μL, 360 μL, 270 μL, and 200 μL).

The powders and/or respirable dry particles preferably may be administered with low inhalation energy. In order to relate the dispersion of powder at different inhalation flow rates, volumes, and from inhalers of different resistances, the energy required to perform the inhalation maneuver may be calculated. Inhalation energy can be calculated from the equation E=R²Q²V where E is the inhalation energy in Joules, R is the inhaler resistance in kPa^{1/2}/LPM, Q is the steady flow rate in L/min and V is the inhaled air volume in L.

Healthy adult populations are predicted to be able to achieve inhalation energies ranging from 2.9 Joules for comfortable inhalations to 22 Joules for maximum inhalations by using values of peak inspiratory flow rate (PIFR) measured by Clarke et al. (Journal of Aerosol Med, 6(2), 99-110, 1993) for the flow rate Q from two inhaler resistances of 0.02 and 0.055 kPa½/LPM, with a inhalation volume of 2L based on both FDA guidance documents for dry powder inhalers and on the work of Tiddens et al. (Journal of Aerosol Med, 19(4), 456-465, 2006) who found adults averaging 2.2L inhaled volume through a variety of dry powder inhalers (DPIs).

An advantage of aspects of the invention is the production of powders that disperse well across a wide range of flow rates and are relatively flow rate independent. In certain aspects, the dry particles and powders of the invention enable the use of a simple, passive DPI for a wide patient population.

In preferred aspects, the respirable dry powder comprises respirable dry particles that characterized by:
1. VMGD at 1 bar as measured using a HELOS/RODOS system between 0.5 microns and 10 microns, preferably between 1 microns and 7 microns, between 1 microns and 5 microns, or between 1 microns and 3 microns;
2. 1 bar/4 bar of 1.6 or less, preferably less than 1.5, less than 1.4, less than 1.3, less than 1.2, or less than 1.1; and
3. tap density of about 0.2 g/cm³ to about 1.2 g/cm³, 0.3 g/cm³ to about 1.0 g/cm³, 0.4 g/cm³ to about 1.0 g/cm³, 0.5 g/cm³ to about 1.0 g/cm³, or between about 0.6 g/cm³ and about 0.9 g/cm³.

In other preferred aspects, the respirable dry powder comprises respirable dry particles that characterized by:
1. VMGD at 1 bar as measured using a HELOS/RODOS system between 0.5 microns and 10 microns, preferably between 1 microns and 7 microns, between 1 microns and 5 microns, or between 1 microns and 3 microns;
2. 1 bar/4 bar of 1.6 or less, preferably less than 1.5, less than 1.4, less than 1.3, less than 1.2 or less than 1.1; and
3. MMAD between 0.5 and 6.0, between 1.0 and 5.0, or between 1.0 and 3.0. In such aspects, the dry powder preferably comprises respirable dry particles that contain DHE mesylate, sodium chloride, leucine (e.g., L-leucine), and mannitol; wherein the DHE mesylate is present in an amount of between about 1% and about 30% by weight; the sodium chloride is present in an amount of between about 2% and about 25% by weight; the mannitol is present in an amount of between about 35% and about 75% by weight; and the leucine is present in an amount of between about 5% and about 35% by weight; or more preferably, wherein the DHE mesylate is present in an amount of between about 1% and about 15% by weight; the sodium chloride is present in an amount of between about 4% and about 14% by weight; the mannitol is present in an amount of between about 55% and about 75% by weight; and the leucine is present in an amount of between about 12% and about 25% by weight; wherein all percentages are weight percentages on a dry basis and all the components of the respirable dry particles amount to 100%.

In other preferred aspects, the respirable dry powder comprises respirable dry particles that characterized by:
1. VMGD at 1 bar as measured using a HELOS/RODOS system between 0.5 microns and 10 microns, preferably between 1 microns and 7 microns, between 1 microns and 5 microns, or between 1 microns and 3 microns;
2. 1 bar/4 bar of 1.6 or less, preferably less than 1.5, less than 1.4, less than 1.3, less than 1.2 or less than 1.1; and
3. FPF_TD<5.0 μm of at least 30%, at least 40%, at least 50% or at least 60%. In such aspects, the dry powder preferably comprises respirable dry particles that contain DHE mesylate, sodium chloride, leucine (e.g., L-leucine), and mannitol; wherein the DHE mesylate is present in an amount of between about 1% and about 30% by weight; the sodium chloride is present in an amount of between about 2% and about 25% by weight; the mannitol is present in an amount of between about 35% and about 75% by weight; and the leucine is present in an amount of between about 5% and about 35% by weight; or more preferably, wherein the DHE mesylate is present in an amount of between about 1% and about 15% by weight; the sodium chloride is present in an amount of between about 4% and about 14% by weight; the mannitol is present in an amount of between about 55% and about 75% by weight; and the leucine is present in an amount of between about 12% and about 25% by weight; wherein all percentages are weight percentages on a dry basis and all the components of the respirable dry particles amount to 100%.

The respirable dry particles and dry powders described herein are suitable for inhalation. The respirable dry particles may be fabricated with the appropriate material, surface roughness, diameter and density for localized delivery to selected regions of the respiratory system such as the deep lung or upper or central airways. For example, higher density or larger respirable dry particles may be used for upper airway delivery, or a mixture of varying size respirable dry particles in a sample, provided with the same or a different formulation, may be administered to target different regions of the lung in one administration.

Because the respirable dry powders and respirable dry particles described herein contain salts, they may be hygroscopic. Accordingly, it is desirable to store or maintain the respirable dry powders and respirable dry particles under conditions to prevent hydration of the powders. For example, if it is desirable to prevent hydration, the relative humidity of the storage environment should be less than 75%, less than 60%, less than 50%, less than 40%, less than 35%, less than 30%, less than 25%, less than 20%, less than 15%, less than 10%, or less than 5% humidity. In other embodiments, the storage environment should be between 20% to 40%, between 25% to 35%, about 30%, between 10% to 20%, or about 15% humidity. The respirable dry powders and respirable dry particles can be packaged (e.g., in sealed capsules, blisters, vials) under these conditions.

In preferred embodiments, the respirable dry powders or respirable dry particles of the invention possess aerosol characteristics that permit effective delivery of the respirable dry particles to the respiratory system without the use of a propellant (e.g., using a passive dry powder inhaler). In preferred embodiments, the respirable dry powders are not combined with a propellant, e.g., for administration with a pressurized device.

In an aspect of the invention, the respirable dry particles and dry powder are contained in a receptacle (or unit dose container), such as a blister, capsule, reservoir, vial, or the like. In a preferred aspect, the respirable dry powder is contained in a blister. In another preferred aspect, the respirable dry powder is contained in a capsule.

The receptacle can contain any desired amount of the respirable dry powder. For example, the receptacle can contain about 20 mg of the dry powder or less, e.g., about 10 mg or less, or about 5 mg or less, e.g., between about 1 mg and about 20 mg, between about 1 mg and about 10 mg, between about 1 mg and about 5 mg, between about 5 mg and about 10 mg, between about 10 mg and about 20 mg, between about 10 mg and about 15 mg, between about 15 mg and about 20 mg, e.g., about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, or about 20 mg, of the dry powder. In preferred aspect, the receptacle contains between about 1 mg to about 20 mg of the dry powder, e.g., between about 2 mg to about 15 mg, or between about 2 mg to about 10 mg of the dry powder, e.g., about 2 mg, 3 mg, 4 mg, 5 mg, 6 mg, 7 mg, 8 mg, 9 mg, or 10 mg of the dry powder. In an embodiment, the receptacle contains about 5 mg of the dry powder.

The dry powder can fill the receptacle, or the receptacle can be at least 2% full, at least 5% full, at least 10% full, at least 20% full, at least 30% full, at least 40% full, at least 50% full, at least 60% full, at least 70% full, at least 80% full, or at least 90% full. The receptacle can be a capsule (e.g., size 000, 00, 00E, 0, 1, 2, 3, and 4, with respective volumetric capacities of 1.37 mL, 950 μL, 770 μL, 680 μL, 480 μL, 360 μL, 270 μL, and 200 μL). The capsule can be at least about 2% full, at least about 5% full, at least about 10% full, at least about 20% full, at least about 30% full, at least about 40% full, or at least about 50% full. The receptacle can be a blister. The blister can be packaged as a single blister or as part of a set of blisters, for example, 7 blisters, 14 blisters, 28 blisters or 30 blisters. The one or more blister can be preferably at least 30% full, at least 50% full or at least 70% full.

The receptacle can contain any nominal dose of DHE or a salt, hydrate, or polymorph thereof (e.g., DHE mesylate). In preferred aspect, the receptacle contains a nominal dose of DHE mesylate of between about 50 μg and about 5000 μg, or between about 75 μg and about 2000 μg DHE mesylate, or between about 100 μg and about 1000 μg DHE mesylate. For example, the receptacle can contain between about 50 μg and about 200 μg, between about 100 μg and about 200 μg, between about 250 μg and about 750 μg, between about 300 μg and about 600 μg, or about 50 μg, about 75 μg, about 100 μg, about 125 μg, about 150 μg, about 175 μg, about 200 μg, about 250 μg, about 300 μg, about 350 μg, about 400 μg, about 450 μg, about 500 μg, about 550 μg, about 600 μg, about 650 μg, about 700 μg, about 750 μg, about 800 μg, about 850 μg, about 900 μg, about 950 μg, about 1000 μg, about 1250 μg, or about 1500 μg, of DHE mesylate.

The receptacle, dry powder and/or respirable dry particles described and characterized herein can be contained by any suitable device, such a dry-powder inhaler (DPI), including any DPI described herein. In some embodiments, the DPI is a passive DPI. The DPI can be any suitable DPI for containing a receptacle containing the dry powder and/or administering the dry powder, such as a capsule-based DPI, a blister-based DPI, or a reservoir-based DPI. In some embodiments, may be contained in a dry powder inhaler (DPI). In a preferred aspect, the DPI is a capsule-based DPI. More preferably, the DPI is selected from the RS01 family of dry powder inhalers (Plastiape S.p.A., Italy). More preferably, the dry powder inhaler is selected from the RS01 HR, RS01 UHR or the RS01 UHR2.

### Methods for Preparing Dry Powders and Dry Particles

The respirable dry particles and dry powders can be prepared using any suitable method. Many suitable methods for preparing respirable dry powders and particles are conventional in the art, and include single and double emulsion solvent evaporation, spray drying, spray freeze drying, milling (e.g., jet milling), blending, solvent extraction, solvent evaporation, phase separation, simple and complex coacervation, interfacial polymerization, suitable methods that involve the use of supercritical carbon dioxide (CO₂), sonocrystalliztion, nanoparticle aggregate formation and other suitable methods, including combinations thereof. Respirable dry particles can be made using methods for making microspheres or microcapsules known in the art. These methods can be employed under conditions that result in the formation of respirable dry particles with desired aerodynamic properties (e.g., aerodynamic diameter and geometric diameter). If desired, respirable dry particles with desired properties, such as size and density, can be selected using suitable methods, such as sieving.

Suitable methods for selecting respirable dry particles with desired properties, such as size and density, include wet sieving, dry sieving, and aerodynamic classifiers (such as cyclones).

The respirable dry particles are preferably spray dried. Suitable spray drying techniques are described, for example, by K. Masters in “Spray Drying Handbook”, John Wiley & Sons, New York (1984). Generally, during spray drying, heat from a hot gas such as heated air or nitrogen is used to evaporate a solvent from droplets formed by atomizing a continuous liquid feed. If desired, the spray drying or other instruments, e.g., jet milling instrument, used to prepare the dry particles can include an inline geometric particle sizer that determines a geometric diameter of the respirable dry particles as they are being produced, and/or an inline aerodynamic particle sizer that determines the aerodynamic diameter of the respirable dry particles as they are being produced.

For spray drying, solutions, emulsions or suspensions that contain the components of the dry particles to be produced in a suitable solvent (e.g., aqueous solvent, organic solvent, aqueous-organic mixture or emulsion) are distributed to a drying vessel via an atomization device. For example, a nozzle or a rotary atomizer may be used to distribute the solution or suspension to the drying vessel. For example, a rotary atomizer having a 4- or 24-vaned wheel may be used. Examples of suitable spray dryers that can be outfitted with either a rotary atomizer or a nozzle, include a Mobile Minor Spray Dryer or the Model PSD-1, both manufactured by GEA Group (Niro, Denmark), Büchi B-290 Mini Spray Dryer (BÜCHI Labortechnik AG, Flawil, Switzerland), ProCepT Formatrix R&D spray dryer (ProCepT nv, Zelzate, Belgium), among several other spray dryer options. Actual spray drying conditions will vary depending, in part, on the composition of the spray drying solution or suspension and material flow rates. The person of ordinary skill will be able to determine appropriate conditions based on the compositions of the solution, emulsion or suspension to be spray dried, the desired particle properties and other factors. In general, the inlet temperature to the spray dryer is about 90° C. to about 300° C., and preferably is about 220° C. to about 285° C. The spray dryer outlet temperature will vary depending upon such factors as the feed temperature and the properties of the materials being dried. If desired, the respirable dry particles that are produced can be fractionated by volumetric size, for example, using a sieve, or fractioned by aerodynamic size, for example, using a cyclone, and/or further separated according to density using techniques known to those of skill in the art.

To prepare the respirable dry particles of the invention, generally, a solution, emulsion or suspension that contains the desired components of the dry powder (i.e., a feed stock) is prepared and spray dried under suitable conditions. Preferably, the dissolved or suspended solids concentration in the feed stock is at least about 1 g/L, at least about 2 g/L, at least about 5 g/L, at least about 10 g/L, at least about 15 g/L, at least about 20 g/L, at least about 30 g/L, at least about 40 g/L, at least about 50 g/L, at least about 60 g/L, at least about 70 g/L, at least about 80 g/L, at least about 90 g/L, or at least about 100 g/L. The feed stock can be provided by preparing a single solution or suspension by dissolving or suspending suitable components (e.g., salts, excipients, other active ingredients) in a suitable solvent. The solvent, emulsion or suspension can be prepared using any suitable methods, such as bulk mixing of dry and/or liquid components or static mixing of liquid components to form a combination. For example, a hydrophilic component (e.g., an aqueous solution) and a hydrophobic component (e.g., an organic solution) can be combined using a static mixer to form a combination. The combination can then be atomized to produce droplets, which are dried to form respirable dry particles. Preferably, the atomizing step is performed immediately after the components are combined in the static mixer. Alternatively, the atomizing step is performed on a bulk mixed solution.

The feedstock, or components of the feedstock, can be prepared using any suitable solvent, such as an organic solvent, an aqueous solvent or mixtures thereof. Suitable organic solvents that can be employed include but are not limited to alcohols such as, for example, ethanol, methanol, propanol, isopropanol, butanols, and others. Other organic solvents include but are not limited to tetrahydrofuran (THF), perfluorocarbons, dichloromethane, chloroform, ether, ethyl acetate, methyl tert-butyl ether, dimethylformamide, and others. Co-solvents that can be employed include an aqueous solvent and an organic solvent, such as, but not limited to, the organic solvents as described above. Aqueous solvents include water and buffered solutions.

The feedstock or components of the feedstock can have any desired pH, viscosity or other properties. If desired, a pH buffer can be added to the solvent or co-solvent or to the formed mixture. Generally, the pH of the mixture ranges from about 3 to about 8. Methanesulfonic acid can be used to acidify water for use as a solvent.

Respirable dry particles and dry powders can be fabricated and then separated, for example, by filtration or centrifugation by means of a cyclone, to provide a particle sample with a preselected size distribution. For example, greater than about 30%, greater than about 40%, greater than about 50%, greater than about 60%, greater than about 70%, greater than about 80%, or greater than about 90% of the respirable dry particles in a sample can have a diameter within a selected range. The selected range within which a certain percentage of the respirable dry particles fall can be, for example, any of the size ranges described herein, such as between about 0.1 to about 3 microns VMGD, or between 0.5 to about 5 micron VMGD.

The invention also relates to respirable dry powders or respirable dry particles produced by preparing a feedstock solution, emulsion or suspension and spray drying the feedstock according to the methods described herein. The feedstock can be prepared, for example, using dihydroergotamine (DHE) or a salt, hydrate, or polymorph thereof, such as dihydroergotamine mesylate, in an amount of about 1% to 99% by weight (e.g., of total solutes used for preparing the feedstock), a monovalent metal cation salt, such as sodium chloride or potassium chloride, in an amount of about 1% to 99% by weight (e.g., of total solutes used for preparing the feedstock), one or more excipients, such as leucine (e.g., L-leucine), mannitol, or both, with each excipient in an amount of about 1% to 99% by weight (e.g., of total solutes used for preparing the feedstock), and one or more suitable solvents for dissolution of the solute and formation of the feedstock. In a preferred embodiment, the feedstock is prepared using DHE mesylate in an amount of about 1% to about 25% by weight of total solutes used for preparing the feedstock (e.g., between about 1% to about 15%, e.g., about 3%, about 5%, or about 10%); sodium chloride in an amount of about 3% to about 15% by weight of total solutes used for preparing the feedstock (e.g., about 9% or about 9.7%); leucine (e.g., L-leucine) in an amount of about 12% to about 26% by weight of total solutes used for preparing the feedstock (e.g., about 18% or about 19.4%); and mannitol in an amount of about 57% to about 74% by weight of total solutes used for preparing the feedstock (e.g., about 63% or about 67.9%); and one or more suitable solvents for dissolution of the solute and formation of the feedstock (e.g., a mixture of dimethylformamide and water; e.g., a 3:7 mixture of DMF /water).

Any suitable method can be used for mixing the solutes and solvents to prepare feedstocks (e.g., static mixing, bulk mixing). If desired, additional components that cause or facilitate the mixing can be included in the feedstock. For example, carbon dioxide produces fizzing or effervescence and thus can serve to promote physical mixing of the solute and solvents. Various salts of carbonate or bicarbonate can promote the same effect that carbon dioxide produces and, therefore, can be used in preparation of the feedstocks of the invention.

In an embodiment, the respirable dry powders or respirable dry particles of the invention can be produced through an ion exchange reaction. In certain embodiments of the invention, two saturated or sub-saturated solutions are fed into a static mixer in order to obtain a saturated or supersaturated solution post-static mixing. Preferably, the post-mixed solution is supersaturated. The post-mixed solution may be supersaturated in all components or supersaturated in one, two, or three of the components.

The two solutions may be aqueous or organic. When the active agent (e.g., DHE or a salt, hydrate, or polymorph thereof) is dissolved in an organic solvent, then one feed solution may be organic while the other one may be aqueous, or both feed solutions may be organic. The post-static mixing solution is then fed into the atomizing unit of a spray dryer. In a preferable embodiment, the post-static mixing solution is immediately fed into the atomizer unit. Some examples of an atomizer unit include a two-fluid nozzle, a rotary atomizer, or a pressure nozzle. Preferably, the atomizer unit is a two-fluid nozzle. In one embodiment, the two-fluid nozzle is an internally mixing nozzle, meaning that the gas impinges on the liquid feed before exiting to most outward orifice. In another embodiment, the two-fluid nozzle is an externally mixing nozzle, meaning that the gas impinges on the liquid feed after exiting the most outward orifice.

### Characterization of Dry Powders and Dry Particles

The diameter of the respirable dry particles, for example, their VMGD, can be measured using an electrical zone sensing instrument such as a Multisizer IIe, (Coulter Electronic, Luton, Beds, England), or a laser diffraction instrument such as a HELOS system (Sympatec, Princeton, NJ) or a Mastersizer system (Malvern, Worcestershire, UK). Other instruments for measuring particle geometric diameter are well known in the art. The diameter of respirable dry particles in a sample will range depending upon factors such as particle composition and methods of synthesis. The distribution of size of respirable dry particles in a sample can be selected to permit optimal deposition within targeted sites within the respiratory system.

Experimentally, aerodynamic diameter can be determined using time of flight (TOF) measurements. For example, an instrument such as the Aerosol Particle Sizer (APS) Spectrometer (TSI Inc., Shoreview, MN) can be used to measure aerodynamic diameter. The APS measures the time taken for individual respirable dry particles to pass between two fixed laser beams.

Aerodynamic diameter also can be experimentally determined directly using conventional gravitational settling methods, in which the time required for a sample of respirable dry particles to settle a certain distance is measured. Indirect methods for measuring the mass median aerodynamic diameter include the Andersen Cascade Impactor (ACI) and the multi-stage liquid impinger (MSLI) methods. The methods and instruments for measuring particle aerodynamic diameter are well known in the art.

Tap density is a measure of the envelope mass density characterizing a particle. The envelope mass density of a particle of a statistically isotropic shape is defined as the mass of the particle divided by the minimum sphere envelope volume within which it can be enclosed. Features which can contribute to low tap density include irregular surface texture, high particle cohesiveness and porous structure. Tap density can be measured by using instruments known to those skilled in the art such as the Dual Platform Microprocessor Controlled Tap Density Tester (Vankel, NC), a GeoPyc™ instrument (Micrometrics Instrument Corp., Norcross, GA), or SOTAX Tap Density Tester model TD2 (SOTAX Corp., Horsham, PA). Tap density can be determined using the method of USP Bulk Density and Tapped Density, United States Pharmacopeia convention, Rockville, MD, 10th Supplement, 4950-4951, 1999.

Fine particle fraction can be used as one way to characterize the aerosol performance of a dispersed powder. Fine particle fraction describes the size distribution of airborne respirable dry particles. Gravimetric analysis, using a Cascade Impactor, is one method of measuring the size distribution, or fine particle fraction, of airborne respirable dry particles. The Andersen Cascade Impactor (ACI) is an eight-stage Impactor that can separate aerosols into nine distinct fractions based on aerodynamic size. The size cutoffs of each stage are dependent upon the flow rate at which the ACI is operated. The ACI is made up of multiple stages consisting of a series of nozzles (i.e., a jet plate) and an impaction surface (i.e., an impaction disc). At each stage an aerosol stream passes through the nozzles and impinges upon the surface. Respirable dry particles in the aerosol stream with a large enough inertia will impact upon the plate. Smaller respirable dry particles that do not have enough inertia to impact on the plate will remain in the aerosol stream and be carried to the next stage. Each successive stage of the ACI has a higher aerosol velocity in the nozzles so that smaller respirable dry particles can be collected at each successive stage. Specifically, an eight-stage ACI is calibrated so that the fraction of powder that is collected on stage 2 and all lower stages including the final collection filter is composed of respirable dry particles that have an aerodynamic diameter of less than 4.4 microns. The airflow at such a calibration is approximately 60 L/min.

Another method that can be used for measuring size distribution is using a Next Generation Impactor (NGI). The NGI consists of seven stages that separate aerosol particles based on inertial impaction and can be operated at a variety of air flow rates. At each stage, the aerosol stream passes through a set of nozzles and impinges on a corresponding impaction surface. Particles having small enough inertia will continue with the aerosol stream to the next stage, while the remaining particles will impact upon the surface. At each successive stage, the aerosol passes through nozzles at a higher velocity and aerodynamically smaller particles are collected on the plate. After the aerosol passes through the final stage, a micro-orifice collector collects the smallest particles that remain. Gravimetric and/or chemical analyses can then be performed to determine the particle size distribution.

If desired, a two-stage collapsed ACI can also be used to measure fine particle fraction. The two-stage collapsed ACI consists of only the top two stages 0 and 2 of the eight-stage ACI, as well as the final collection filter, and allows for the collection of two separate powder fractions. Specifically, a two-stage collapsed ACI is calibrated so that the fraction of powder that is collected on stage two is composed of respirable dry particles that have an aerodynamic diameter of less than 5.6 microns and greater than 3.4 microns. The fraction of powder passing stage two and depositing on the final collection filter is thus composed of respirable dry particles having an aerodynamic diameter of less than 3.4 microns. The airflow at such a calibration is approximately 60 L/min.

The FPF(<5.6) has been demonstrated to correlate to the fraction of the powder that is able to reach the lungs of the patient, while the FPF(<3.4) has been demonstrated to correlate to the fraction of the powder that reaches the deep lung of a patient. These correlations provide a quantitative indicator that can be used for particle optimization.

Emitted dose can be determined using the method of USP Section 601 Aerosols, Metered-Dose Inhalers and Dry Powder Inhalers, Delivered-Dose Uniformity, Sampling the Delivered Dose from Dry Powder Inhalers, United States Pharmacopeia convention, Rockville, MD, 13^{th} Revision, 222-225, 2007. This method utilizes an in vitro device set up to mimic patient dosing.

An ACI can be used to approximate the emitted dose, which herein is called gravimetric recovered dose and analytical recovered dose. “Gravimetric recovered dose” is defined as the ratio of the powder weighed on all stage filters of the ACI to the nominal dose. “Analytical recovered dose” is defined as the ratio of the powder recovered from rinsing and analyzing all stages, all stage filters, and the induction port of the ACI to the nominal dose. The FPF_TD (<5.0) is the ratio of the interpolated amount of powder depositing below 5.0 μm on the ACI to the nominal dose. The FPF_RD (<5.0) is the ratio of the interpolated amount of powder depositing below 5.0 μm on the ACI to either the gravimetric recovered dose or the analytical recovered dose.

Another way to approximate emitted dose is to determine how much powder leaves its container, e.g. capture or blister, upon actuation of a dry powder inhaler (DPI). This takes into account the percentage leaving the capsule, but does not take into account any powder depositing on the DPI. The emitted powder mass is the difference in the weight of the capsule with the dose before inhaler actuation and the weight of the capsule after inhaler actuation. This measurement can be called the capsule emitted powder mass (CEPM) or sometimes termed “shot-weight”.

A Multi-Stage Liquid Impinger (MSLI) is another device that can be used to measure fine particle fraction. The MSLI operates on the same principles as the ACI, although instead of eight stages, MSLI has five. Additionally, each MSLI stage consists of an ethanol-wetted glass frit instead of a solid plate. The wetted stage is used to prevent particle bounce and re-entrainment, which can occur when using the ACI.

The Next Generation Pharmaceutical Impactor (NGI) is a particle-classifying cascade impactor for testing metered-dose, dry-powder, and similar inhaler devices.

The geometric particle size distribution can be measured for the respirable dry powder after being emitted from a dry powder inhaler (DPI) by use of a laser diffraction instrument such as the Malvern Spraytec. With the inhaler adapter in the close-bench configuration, an airtight seal is made to the DPI, causing the outlet aerosol to pass perpendicularly through the laser beam as an internal flow. In this way, known flow rates can be drawn through the DPI by vacuum pressure to empty the DPI. The resulting geometric particle size distribution of the aerosol is measured by the photodetectors with samples typically taken at 1000 Hz for the duration of the inhalation and the DV₅₀, GSD, FPF<5.0 μm measured and averaged over the duration of the inhalation.

Water content of the respirable dry powder or respirable dry particles can be measured by a Karl Fisher titration machine, or by a Thermogravimetric Analysis or Thermal Gravimetric Analysis (TGA). Karl Fischer titration uses coulometric or volumetric titration to determine trace amounts of water in a sample. TGA is a method of thermal analysis in which changes in weight of materials are measured as a function of temperature (with constant heating rate), or as a function of time (with constant temperature and/or constant mass loss). TGA may be used to determine the water content or residual solvent content of the material being tested.

The invention also relates to a respirable dry powder or respirable dry particles produced using any of the methods described herein.

The respirable dry particles of the invention can also be characterized by the physicochemical stability of the components that the respirable dry particles comprise. The physicochemical stability of the components can affect important characteristics of the respirable particles including shelf-life, proper storage conditions, acceptable environments for administration, biological compatibility, and effectiveness. Chemical stability can be assessed using techniques well known in the art. One example of a technique that can be used to assess chemical stability is reverse phase high performance liquid chromatography (RP-HPLC).

If desired, the respirable dry particles and dry powders described herein can be further processed to increase stability. An important characteristic of pharmaceutical dry powders is whether they are stable at different temperature and humidity conditions. Unstable powders will absorb moisture from the environment and agglomerate, thus altering particle size distribution of the powder.

### Therapeutic Use and Methods

The respirable dry powders and respirable dry particles of the present invention are for administration to the respiratory tract. For example, the respirable dry powders described herein can be administered to a subject in need thereof via inhalation (e.g., oral inhalation) to treat a migraine or a symptom thereof. In some embodiments, the respirable dry powders described herein can be administered to a subject in need thereof to treat a headache or a symptom thereof (e.g., a cluster headache).

The respirable dry powder can be used to treat a migraine or a symptom thereof. The migraine can include any form of migraine, for example a migraine with aura, a migraine without aura, a cluster migraine, basilar migraine, episodic migraine, chronic migraine, refractory migraine, hemiplegic migraine, triptan-resistant migraine, pediatric migraine, status migraine, migraine with allodynia, menstrual migraine, migraine-upon-awakening, or rapid-onset migraine.

In some embodiments, administering an effective amount of the respirable dry powder can provide relief (e.g., complete or partial relief) of one or more symptoms of a migraine. Examples of migraine symptoms include pain, nausea, phonophobia, and photophobia. In some aspects, administering an effective amount of the respirable dry powder lessens the intensity of pain, nausea, phonophobia, photophobia, or a combination thereof, in a subject.

Administering an effective amount of the respirable dry powder to a subject in need thereof can provide relief (e.g., partial or complete relief) of a migraine or a symptom thereof within a period of about 2 hours or less following administration, e.g., within about 1.5 hours, about 1 hour, about 0.5 hours, or less, following administration of the dry powder. In a preferred aspect, administering an effective amount of the respirable dry powder to a subject in need thereof provides relief (e.g., partial or complete relief) of a migraine or a symptom thereof within a period of about 1 hour. In another preferred aspect, administering an effective amount of the respirable dry powder to a subject in need thereof provides relief (e.g., partial or complete relief) of a migraine or a symptom thereof within a period of about 0.75 hours. In another preferred aspect, administering an effective amount of the respirable dry powder to a subject in need thereof provides relief (e.g., partial or complete relief) of a migraine or a symptom thereof within a period of about 0.5 hours.

Administering an effective amount of the respirable dry powder to a subject in need thereof can also provide sustained relief (e.g., sustained partial or complete relief) of a migraine or a symptom thereof. For example, following administration of the respirable dry powder to a subject in need thereof, relief of a migraine or a symptom thereof can be sustained for a period of at least about 6 hours, at least about 8 hours, at least about 10 hours, at least about 12 hours, at least about 18 hours, at least about 24 hours, at least about 36 hours, or longer.

Relief of a migraine or a symptom thereof can be measured using any appropriate symptom intensity or functional disability scale, such as a scale recommended by the International Headache Society (IHS); a 100-mm Visual Analogue Scale; an 11-point numerical rating scale; and/or any another appropriate method, such as a method described in Diener, H.-C. Cephalagia (2019), 39(6):687-710. For example, relief of a migraine or a symptom thereof can be determined by pain freedom at 2 hours after treatment. Alternatively or in addition, relief of a migraine or a symptom thereof can be determined by a reduction in a headache intensity score using a 4-point scale (e.g., where 0=no headache; 1=mild headache; 2=moderate headache; and 3=severe headache), e.g., a reduction from a score of 3 to 2, 3 to 1, 3 to 0, 2 to 1, 2 to 0, or 1 to 0.

The dry powder can be administered to a subject in need thereof at any stage of a migraine, and provide effective relief of the migraine or a symptom thereof. For example, the dry powder can be administered during the prodrome, aura, migraine attack, or postdrome stage of the migraine. In an embodiment, the dry powder is administered to a subject in need thereof during the aura stage of a migraine. In an embodiment, the dry powder is administered to a subject in need thereof during the migraine attack stage of a migraine.

The administration of DHE can be associated with certain side-effects. Common side-effects of DHE (e.g., following intravenous administration) can include emesis, nausea, and chest tightness. Other side-effects can include cardiovascular effects (e.g., blood pressure instability, arterial constriction, hypertension, or cardiac valvulopathy), paraesthesia, anxiety, dyspnea, headache, diarrhea, skin rash, drowsiness, dizziness, flushing, increased sweating, retroperitoneal fibrosis, and pleural fibrosis (Silberstein, S., supra; Saper, J., supra; D.H.E. 45® [package insert]. Aliso Viejo, CA: Valeant Pharmaceuticals). An advantage of the invention is that administering an effective amount the dry powder disclosed herein via inhalation to a subject in need thereof can reduce or eliminate side-effects that are commonly associated with the administration of an effective amount of DHE by another route (e.g., intravenous).

In a preferred aspect, the administration of the dry powder to a subject in need thereof does not cause emesis, and/or does not require also administering an anti-emetic drug to the subject, such as an anti-emetic typically administered to a subject receiving intravenous DHE (e.g., ondansetron, granisetron, metoclopramide, promethazine, prochlorperazine, domperidone, or aprepitant).

Without wishing to be bound by theory, it is believed that inhalation of an effective amount of the dry powder disclosed herein results in superior pharmacokinetics with respect to conventional or routes of DHE administration (e.g., intravenous DHE), that contribute to a rapid therapeutic effect, e.g., partial or complete relief of a symptom within 1 hour or less (e.g., about 30 minutes) and relatively low incidence or severity of side-effects, for example (i) a time to peak plasma concentration (Tₘₐₓ) of about 20 minutes or less; (ii) a peak plasma concentration (Cₘₐₓ) of between about 2000 pg/mL to about 6000 pg/mL; (iii) an AUC_{inf} between about 5000 pg*h/mL to about 10,000 pg*h/mL; and/or (iv) an elimination half-life (t_{1/2}) of between about 6 hours and about 14 hours (e.g., between about 8 hours and about 12 hours). For example, administration of an effective amount of the presently disclosed dry powder to a subject in need thereof can relieve the migraine or a symptom thereof within about 30 minutes, without the occurrence of emesis.

In some embodiments, administering an effective amount of the dry powder to a subject in need thereof results in a time to peak plasma concentration (Tₘₐₓ) of DHE of less than about 30 minutes. For example, administering an effective amount of the dry powder to a subject in need thereof can result in a time to peak plasma concentration (Tₘₐₓ) of DHE of less than about 25 minutes, less than about 20 minutes, less than about 15 minutes, less than about 10 minutes, less than about 5 minutes, less than about 4 minutes, less than about 3 minutes, less than about 2 minutes, or less than about 1 minute. In some preferred aspects, administering an effective amount of the dry powder to a subject in need thereof results in a time to peak plasma concentration (Tₘₐₓ) of DHE of less than about 15 minutes. In other preferred aspects, administering an effective amount of the dry powder to a subject in need thereof results in a time to peak plasma concentration (Tₘₐₓ) of DHE of less than about 10 minutes. In other preferred aspects, administering an effective amount of the dry powder to a subject in need thereof results in a time to peak plasma concentration (Tₘₐₓ) of DHE of less than about 5 minutes.

In some aspects, administering an effective amount of the dry powder to a subject in need thereof results in an elimination half-life (t_{1/2}) of DHE of between about 6 hours and about 14 hours. For example, the t_{1/2} can be between about 6 hours and about 8 hours, between about 7 hours and about 9 hours, between about 8 hours and about 10 hours, between about 9 hours and about 11 hours, between about 10 hours and about 12 hours, or between about 11 hours and about 13 hours, or about 7 hours, about 8 hours, about 9 hours, about 10 hours, about 11 hours, or about 12 hours.

Administering an effective amount of the dry powder to a subject in need thereof can results in a peak plasma concentration (Cₘₐₓ) of DHE of between about 500 pg/mL and about 15,000 pg/mL, e.g., between about 500 pg/mL and about 14,000 pg/mL, between about 2000 pg/mL and about 11,000 pg/mL, between about 500 pg/mL and about 8000 pg/mL, between about 1500 pg/mL and about 7000 pg/mL, or more preferably between about 2000 pg/mL and about 6000 pg/mL, or even more preferably between about 3000 pg/mL and about 5,000 pg/mL. In some embodiments, administering an effective amount of the dry powder to a subject in need thereof results in a Cₘₐₓ of DHE of between about 1000 pg/mL and about 4000 pg/mL, between about 4000 pg/mL and about 8000 pg/mL, between about 2000 pg/mL and about 4000 pg/mL, between about 3000 pg/mL and about 6000 pg/mL, between about 4000 pg/mL and about 6000 pg/mL, or about 1500 pg/mL, about 2000 pg/mL, about 2500 pg/mL, about 3000 pg/mL, about 3500 pg/mL, about 4000 pg/mL, about 4500 pg/mL, about 5000 pg/mL, about 5500 pg/mL, about 6000 pg/mL, or about 6500 pg/mL. In a preferred aspect, administering an effective amount of the dry powder to a subject in need thereof results in a Cₘₐₓ of DHE of between about 2000 pg/mL and about 6000 pg/mL. In a more preferred aspect, administering an effective amount of the dry powder to a subject in need thereof results in a Cₘₐₓ of DHE of between about 3000 pg/mL and about 5000 pg/mL.

In some aspects, administering an effective amount of the dry powder to a subject in need thereof results in an area-under-the-concentration curve from time zero to infinity (AUC_{0-inf}) of between about 1000 pg*hour/mL and about 15,000 pg*hour/mL, or between about 2500 pg*hour/mL and about 12,000 pg*hour/mL, or more preferably between about 5000 pg*hour/mL and about 10,000 pg*hour/mL, or even more preferably between about 7000 pg*hour/mL and about 9000 pg*hour/mL. In some embodiments, administering an effective amount of the dry powder to a subject in need thereof results in an AUC_{0-inf} of between about 1500 pg*hour/mL and about 3000 pg*hour/mL, between about 2000 pg*hour/mL and about 4000 pg*hour/mL, between about 3000 pg*hour/mL and about 5000 pg*hour/mL, between about 5000 pg*hour/mL and about 7000 pg*hour/mL, between about 6000 pg*hour/mL and about 8000 pg*hour/mL, between about 8000 pg*hour/mL and about 10,000 pg*hour/mL, or about 1500 pg*hour/mL, about 2000 pg*hour/mL, about 2500 pg*hour/mL, about 3000 pg*hour/mL, about 3500 pg*hour/mL, about 4000 pg*hour/mL, about 4500 pg*hour/mL, about 5000 pg*hour/mL, about 5500 pg*hour/mL, about 6000 pg*hour/mL, about 6500 pg*hour/mL, about 7000 pg*hour/mL, about 7500 pg*hour/mL, about 8000 pg*hour/mL, about 8500 pg*hour/mL, about 9000 pg*hour/mL, about 9500 pg*hour/mL, about 10,000 pg*hour/mL, about 10,500 pg*hour/mL, or about 11,000 pg*hour/mL.

In other aspects, administering an effective amount of the dry powder to a subject in need thereof results in an area-under-the-concentration curve from time zero to 48 hours (AUC₀₋₄₈ₕ) of between about 950 pg*hour/mL and about 14,500 pg*hour/mL, or between about 2250 pg*hour/mL and about 11,500 pg*hour/mL, or between about 4500 pg*hour/mL and about 9500 pg*hour/mL. In some embodiments, administering an effective amount of the dry powder to a subject in need thereof results in an AUC₀₋₄₈ₕ of between about 1000 pg*hour/mL and about 2000 pg*hour/mL, between about 2000 pg*hour/mL and about 4000 pg*hour/mL, between about 3000 pg*hour/mL and about 5000 pg*hour/mL, between about 5000 pg*hour/mL and about 7000 pg*hour/mL, between about 6000 pg*hour/mL and about 8000 pg*hour/mL, between about 8000 pg*hour/mL and about 10,000 pg*hour/mL, or about 1500 pg*hour/mL, about 2000 pg*hour/mL, about 2500 pg*hour/mL, about 3000 pg*hour/mL, about 3500 pg*hour/mL, about 4000 pg*hour/mL, about 4500 pg*hour/mL, about 5000 pg*hour/mL, about 5500 pg*hour/mL, about 6000 pg*hour/mL, about 6500 pg*hour/mL, about 7000 pg*hour/mL, about 7500 pg*hour/mL, about 8000 pg*hour/mL, about 8500 pg*hour/mL, about 9000 pg*hour/mL, about 9500 pg*hour/mL, about 10,000 pg*hour/mL, or about 10,500 pg*hour/mL.

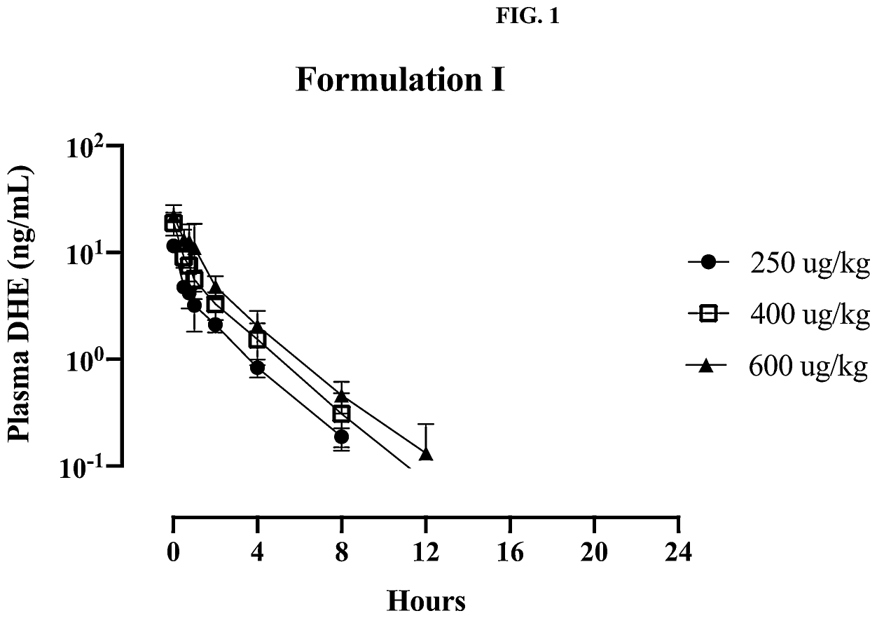

Without wishing to be bound by theory, it is believed that administering an effective amount of a dry powder containing amorphous DHE (e.g., amorphous DHE mesylate) results in a shorter t_{1/2}, a more rapid Tₘₐₓ, and/or a lower AUC, relative to dry powders or other formulations containing crystalline DHE, which can contribute to a more rapid onset of therapeutic effect and lower drug exposure, which should lower incidence and/or severity of undesired side-effects. For example, it was surprisingly discovered that administration of an effective amount of Formulations I and II, which both contain amorphous DHE, by inhalation in a dog model resulted in a t_{1/2} of 1.96 and 2.01 hours, respectively at a dose of 698 μg/kg. On the other hand, administration of crystalline Formulations III and IV at the same dose level resulted in a longer t_{1/2} of 7.00 and 5.68 hours, respectively. This difference between dry powder formulations containing amorphous and crystalline DHE is also apparent by comparing the plasma concentration over time curves in a dog model. As shown in FIGS. 1-4, the plasma concentration over time curves for Formulations I and II are steeper and show that plasma concentration of DHE was minimal by 12 hours for all doses, compared to the curves corresponding to Formulations III and IV which are less steep and include a tail showing that plasma concentrations of DHE persisted beyond 12 hours at higher doses.

Additionally, it is believed that achieving a Cₘₐₓ of DHE in a subject that is reduced relative to Cₘₐₓ achieved following an effective amount of DHE administered intravenously (e.g., 1 mg of intravenous DHE) can contribute to lowering the severity of side-effects, or eliminating side effects, that are commonly experienced following an intravenous dose of with DHE, without compromising efficacy. In some aspects, administering the dry powder to a subject in need thereof can result in a Cₘₐₓ of DHE that is reduced by 10-fold, 20-fold, 30-fold, 40-fold, 50-fold, 60-fold, or more, with respect to Cₘₐₓ following an effective amount of DHE administered intravenously.

The respirable dry particles and dry powders can be administered to the respiratory tract of a subject in need thereof using any suitable method, such as instillation techniques, and/or an inhalation device, such as a dry powder inhaler (DPI) or metered dose inhaler (MDI). A number of DPIs are available, such as, the inhalers disclosed is U.S. Pat. Nos. 4,995,385 and 4,069,819, SPINHALER® (Fisons, Loughborough, U.K.), ROTAHALERS®, DISKHALER® and DISKUS® (GlaxoSmithKline, Research Triangle Technology Park, North Carolina), FLOWCAPSS® (Hovione, Loures, Portugal), INHALATORS® (Boehringer-Ingelheim, Germany), AEROLIZER® (Novartis, Switzerland), high-resistance and low-resistance RS-01 (Plastiape, Italy), and others known to those skilled in the art.

The following scientific journal articles are incorporated by reference for their thorough overview of the following dry powder inhaler (DPI) configurations: 1) Single-dose Capsule DPI, 2) Multi-dose Blister DPI, and 3) Multi-dose Reservoir DPI. N. Islam, E. Gladki, “Dry powder inhalers (DPIs)—A review of device reliability and innovation”, International Journal of Pharmaceuticals, 360(2008):1-11. H. Chystyn, “Diskus Review”, International Journal of Clinical Practice, June 2007, 61, 6, 1022-1036. H. Steckel, B. Muller, “In vitro evaluation of dry powder inhalers I: drug deposition of commonly used devices”, International Journal of Pharmaceuticals, 154(1997):19-29. Some representative capsule-based DPI units are RS-01 (Plastiape, Italy), TURBOSPIN® (PH&T, Italy), BREZHALER® (Novartis, Switzerland), AEROLIZER® (Novartis, Switzerland), PODHALER® (Novartis, Switzerland), HANDIHALER® (Boehringer Ingelheim, Germany), AIR® (Civitas, Massachusetts), DOSE ONE® (Dose One, Maine), and ECLIPSE® (Rhone Poulenc Rorer). Some representative unit dose DPIs are CONIX® (3M, Minnesota), CRICKET® (Mannkind, California), DREAMBOAT® (Mannkind, California), OCCORIS® (Team Consulting, Cambridge, UK), SOLIS® (Sandoz), TRIVAIR® (Trimel Biopharma, Canada), and TWINCAPS® (Hovione, Loures, Portugal). Some representative blister-based DPI units are DISKUS® (GlaxoSmithKline (GSK), UK), DISKHALER® (GSK), TAPER DRY® (3M, Minnesota), GEMINI® (GSK), TWINCER® (University of Groningen, Netherlands), ASPIRAIR® (Vectura, UK), ACU-BREATHE® (Respirics, Minnesota, USA), EXUBRA® (Novartis, Switzerland), GYROHALER® (Vectura, UK), OMNIHALER® (Vectura, UK), MICRODOSE® (Microdose Therapeutix, USA), MULTIHALER® (Cipla, India) PROHALER® (Aptar), TECHNOHALER® (Vectura, UK), and XCELOVAIR® (Mylan, Pennsylvania). Some representative reservoir-based DPI units are CLICKHALER® (Vectura), NEXT DPI® (Chiesi), EASYHALER® (Orion), NOVOLIZER® (Meda), PULMOJET® (sanofi-aventis), PULVINAL® (Chiesi), SKYEHALER® (Skyepharma), DUOHALER® (Vectura), TAIFUN® (Akela), FLEXHALER® (AstraZeneca, Sweden), TURBUHALER® (AstraZeneca, Sweden), and TWISTHALER® (Merck), and others known to those skilled in the art.

Generally, inhalation devices (e.g., DPIs) are able to deliver a maximum amount of dry powder or dry particles in a single inhalation, which is related to the capacity of the blisters, capsules (e.g. size 000, 00, 00E, 0, 1, 2, 3, and 4, with respective volumetric capacities of 1.37 mL, 950 μL, 770 μL, 680 μL, 480 μL, 360 μL, 270 μL, and 200 μL) or other means that contain the dry particles or dry powders within the inhaler. Accordingly, delivery of a desired dose or effective amount can involve two or more inhalations. Preferably, each dose that is administered to a subject in need thereof contains an effective amount of respirable dry particles or dry powder and is administered using no more than about 4 inhalations. For example, each dose of respirable dry particles or dry powder can be administered in a single inhalation or 2, 3, or 4 inhalations. The respirable dry particles and dry powders are preferably administered in a single, breath-activated step using a breath-activated DPI. When this type of device is used, the energy of the subject's inhalation both disperses the respirable dry particles and draws them into the respiratory tract.

The respirable dry particles or dry powders can be delivered by inhalation to a desired area within the respiratory tract, as desired. It is well-known that particles with an aerodynamic diameter (MMAD) of about 1 micron to about 3 microns, can be delivered to the deep lung. Larger MMAD, for example, from about 3 microns to about 5 microns can be delivered to the central and upper airways. Therefore, without wishing to be bound by theory, the dry particles of the present disclosure can have a MMAD of about 1 micron to about 5 microns which preferentially deposits more of the therapeutic dose in the central airways than in the upper airways or in the deep lung.

For dry powder inhalers, oral cavity deposition is dominated by inertial impaction and so characterized by the aerosol's Stokes number (DeHaan et al. Journal of Aerosol Science, 35 (3), 309-331, 2003). For equivalent inhaler geometry, breathing pattern and oral cavity geometry, the Stokes number, and so the oral cavity deposition, is primarily affected by the aerodynamic size of the inhaled powder. Hence, factors which contribute to oral deposition of a powder include the size distribution of the individual particles and the dispersibility of the powder. If the MMAD of the individual particles is too large, e.g. above 5 μm, then an increasing percentage of powder will deposit in the oral cavity. Likewise, if a powder has poor dispersibility, it is an indication that the particles will leave the dry powder inhaler and enter the oral cavity as agglomerates. Agglomerated powder will perform aerodynamically like an individual particle as large as the agglomerate, therefore even if the individual particles are small (e.g., MMAD of 5 microns or less), the size distribution of the inhaled powder may have an MMAD of greater than 5 μm, leading to enhanced oral cavity deposition.

Therefore, it is desirable to have a powder in which the particles are small, dense, and dispersible such that the powders consistently deposit in the desired region of the respiratory tract. For example, the respirable dry powders comprising respirable dry particles have a MMAD of 5 microns or less, between about 1 micron and about 5 microns; are highly dispersible (e.g. 1 bar/4 bar or alternatively, 0.5 bar/4 bar of 2.0, and preferably less than 1.5). In some embodiments, the particles are also dense, for example have a high tap density and/or envelope density, such as about 0.4 g/cc or more, about 0.45 g/cc to about 1.2 g/cc, about 0.5 g/cc or more, about 0.55 g/cc or more, about 0.55 g/cc to about 1.0 g/cc, or about 0.6 g/cc to about 1.0 g/cc. The tap density and/or envelop density and MMAD are related theoretically to the VMGD by means of the following formula: MMAD=VMGD*sqrt (envelope density or tap density). If it is desired to deliver a high mass of therapeutic using a fixed volume dosing container, then, particles of higher tap density and/or envelope density are desired.

The respirable dry powders comprising respirable dry particles may also have a tap density of at least about 0.1 g/cm³, e.g., a tap density of greater than 0.2 g/cm³, a tap density of greater than 0.3 g/cm³, a tap density of greater than 0.4 g/cm³, or a tap density of greater than 0.5 g/cm³.

Respirable dry powders and dry particles suitable for use in the methods of the invention can travel through the upper airways (i.e., the oropharynx and larynx), the lower airways, which include the trachea followed by bifurcations into the bronchi and bronchioli, and through the terminal bronchioli which in turn divide into respiratory bronchioli leading then to the ultimate respiratory zone, the alveoli or the deep lung. In one embodiment of the invention, most of the mass of respirable dry powders or particles deposit in the deep lung. In another embodiment of the invention, delivery is primarily to the central airways. In another embodiment, delivery is to the upper airways.

Suitable dosing to provide the desired therapeutic effect can be determined by a clinician based on the severity of the subject's migraines, overall well-being of the subject, and the subject's tolerance to respirable dry particles and dry powders. Based on these and other considerations, a clinician can determine appropriate doses and intervals between doses.

### EXEMPLIFICATION

Materials used in the following Examples and their sources are listed below. Ethanol, sodium chloride, sodium sulfate, polysorbate 80, mannitol, and L-leucine were obtained from Sigma-Aldrich Co. (St. Louis, MO), Spectrum Chemicals (Gardena, CA), Applichem (Maryland Heights, MO), Alfa Aesar (Tewksbury, MA), Thermo Fisher (Waltham, MA), Croda Chemicals (East Yorkshire, United Kingdom) or Merck/Millipore (Darmstadt, Germany). Dihydroergotamine mesylate was obtained from Olon SpA (Italy). Ultrapure (Type II ASTM) water was from a water purification system (Millipore Corp., Billerica, MA), or equivalent.

### Methods

Geometric or Volume Diameter of Suspensions. Volume median diameter (x50 or Dv50), which may also be referred to as volume median geometric diameter (VMGD), of the active agent suspensions was determined using a laser diffraction technique. The equipment consisted of a Horiba LA-950 instrument outfitted with an automated recirculation system for sample handling and removal or a fixed-volume sample cuvette. The sample to a dispersion media, consisting of either deionized water or deionized water with less than 0.5% of a surfactant such as polysorbate 80 or sodium dodecyl sulfate. Ultrasonic energy can be applied to aid in dispersion of the suspension. When the laser transmission was in the correct range, the sample was sonicated for 60 seconds at a setting of 5. The sample was then measured and the particle size distribution reported.

Geometric or Volume Diameter of Dry Powders. Volume median diameter (x50 or Dv50), which may also be referred to as volume median geometric diameter (VMGD), of the dry powder formulations was determined using a laser diffraction technique. The equipment consisted of a HELOS diffractometer and a RODOS dry powder disperser (Sympatec, Inc., Princeton, NJ). The RODOS disperser applies a shear force to a sample of particles, controlled by the regulator pressure (typically set at 1.0 bar with maximum orifice ring pressure) of the incoming compressed dry air. The pressure settings may be varied to vary the amount of energy used to disperse the powder. For example, the dispersion energy may be modulated by changing the regulator pressure from 0.2 bar to 4.0 bar. Powder sample is dispensed from a microspatula into the RODOS funnel. The dispersed particles travel through a laser beam where the resulting diffracted light pattern produced is collected, typically using an R1 lens, by a series of detectors. The ensemble diffraction pattern is then translated into a volume-based particle size distribution using the Fraunhofer diffraction model, on the basis that smaller particles diffract light at larger angles. Using this method, the span of the distribution was also determined per the formula (Dv[90]−Dv[10)/Dv[50]. The span value gives a relative indication of the polydispersity of the particle size distribution.

Aerodynamic Performance via Andersen Cascade Impactor. The aerodynamic properties of the powders dispersed from an inhaler device were assessed with an Mk-II 1 ACFM Andersen Cascade Impactor (Copley Scientific Limited, Nottingham, UK) (ACI). The instrument consists of eight stages that separate aerosol particles based on inertial impaction. At each stage, the aerosol stream passes through a set of nozzles and impinges on a corresponding impaction plate. Particles having small enough inertia will continue with the aerosol stream to the next stage, while the remaining particles will impact upon the plate. At each successive stage, the aerosol passes through nozzles at a higher velocity and aerodynamically smaller particles are collected on the plate. After the aerosol passes through the final stage, a filter collects the smallest particles that remain, called the “final collection filter”. Gravimetric and/or chemical analyses can then be performed to determine the particle size distribution. A short stack cascade impactor, also referred to as a collapsed cascade impactor, is also utilized to allow for reduced labor time to evaluate two aerodynamic particle size cut-points. With this collapsed cascade impactor, stages are eliminated except those required to establish fine and coarse particle fractions. The impaction techniques utilized allowed for the collection of two or eight separate powder fractions. The capsules (HPMC, Size 3; Capsugel Vcaps, Peapack, NJ) were filled with powder to a specific weight and placed in a hand-held, breath-activated dry powder inhaler (DPI) device, the high resistance RS01 DPI or the ultra-high resistance UHR2 DPI (both by Plastiape, Osnago, Italy). The capsule was punctured and the powder was drawn through the cascade impactor operated at a flow rate of 60.0 L/min for 2.0 s. At this flowrate, the calibrated cut-off diameters for the eight stages are 8.6, 6.5, 4.4, 3.3, 2.0, 1.1, 0.5 and 0.3 microns and for the two stages used with the short stack cascade impactor, based on the Andersen Cascade Impactor, the cut-off diameters are 5.6 microns and 3.4 microns. The fractions were collected by placing filters in the apparatus and determining the amount of powder that impinged on them by gravimetric measurements or chemical measurements on an HPLC.

Aerodynamic Performance via Next Generation Impactor. The aerodynamic properties of the powders dispersed from an inhaler device were assessed with a Next Generation Impactor (Copley Scientific Limited, Nottingham, UK) (NGI). The instrument consists of seven stages that separate aerosol particles based on inertial impaction and can be operated at a variety of air flow rates. At each stage, the aerosol stream passes through a set of nozzles and impinges on a corresponding impaction surface. Particles having small enough inertia will continue with the aerosol stream to the next stage, while the remaining particles will impact upon the surface. At each successive stage, the aerosol passes through nozzles at a higher velocity and aerodynamically smaller particles are collected on the plate. After the aerosol passes through the final stage, a micro-orifice collector collects the smallest particles that remain. Gravimetric and/or chemical analyses can then be performed to determine the particle size distribution. The capsules (HPMC, Size 3; Capsugel Vcaps, Peapack, NJ) were filled with powder to a specific weight and placed in a hand-held, breath-activated dry powder inhaler (DPI) device, the high resistance RS01 DPI or the ultra-high resistance RS01 DPI (both by Plastiape, Osnago, Italy). The capsule was punctured and the powder was drawn through the cascade impactor operated at a specified flow rate for 2.0 liters of inhaled air. At the specified flow rate, the cut-off diameters for the stages were calculated. The fractions were collected by placing wetted filters in the apparatus and determining the amount of powder that impinged on them by chemical measurements on an HPLC.

Fine Particle Dose. The fine particle dose indicates the mass of one or more therapeutics in a specific size range and can be used to predict the mass which will reach a certain region in the respiratory tract. The fine particle dose can be measured gravimetrically or chemically via either an ACI or NGI. If measured gravimetrically, since the dry particles are assumed to be homogenous, the mass of the powder on each stage and collection filter can be multiplied by the fraction of therapeutic agent in the formulation to determine the mass of therapeutic. If measured chemically, the powder from each stage or filter is collected, separated, and assayed for example on an HPLC to determine the content of the therapeutic. The cumulative mass deposited on each of the stages at the specified flow rate is calculated and the cumulative mass corresponding to a 5.0 micrometer diameter particle is interpolated. This cumulative mass for a single dose of powder, contained in one or more capsules, actuated into the impactor is equal to the fine particle dose less than 5.0 microns (FPD<5.0 microns).

Mass Median Aerodynamic Diameter. Mass median aerodynamic diameter (MMAD) was determined using the information obtained by the Andersen Cascade Impactor (ACI). The cumulative mass under the stage cut-off diameter is calculated for each stage and normalized by the recovered dose of powder. The MMAD of the powder is then calculated by linear interpolation of the stage cut-off diameters that bracket the 50th percentile. An alternative method of measuring the MMAD is with the Next Generation Impactor (NGI). Like the ACI, the MMAD is calculated with the cumulative mass under the stage cut-off diameter is calculated for each stage and normalized by the recovered dose of powder. The MMAD of the powder is then calculated by linear interpolation of the stage cut-off diameters that bracket the 50th percentile.

Emitted Geometric or Volume Diameter. The volume median diameter (Dv50) of the powder after it is emitted from a dry powder inhaler, which may also be referred to as volume median geometric diameter (VMGD), was determined using a laser diffraction technique via the Spraytec diffractometer (Malvern, Inc.). Powder was filled into size 3 capsules (Vcaps, Capsugel) and placed in a capsule based dry powder inhaler (RS01 Model 7 HR or UHR2, Plastiape, Italy), or DPI, and the DPI sealed inside a cylinder. The cylinder was connected to a positive pressure air source with steady air flow through the system measured with a mass flow meter and its duration controlled with a timer controlled solenoid valve. The exit of the dry powder inhaler was exposed to room pressure and the resulting aerosol jet passed through the laser of the diffraction particle sizer (Spraytec) in its open bench configuration before being captured by a vacuum extractor. The steady air flow rate through the system was initiated using the solenoid valve. A steady air flow rate was drawn through the DPI typically at 60 L/min for a set duration, typically of 2 seconds. Alternatively, the air flow rate drawn through the DPI was sometimes run at 15 L/min, 20 L/min, or 30 L/min. The resulting geometric particle size distribution of the aerosol was calculated from the software based on the measured scatter pattern on the photodetectors with samples typically taken at 1000 Hz for the duration of the inhalation. The Dv50, GSD, FPF<5.0 □m measured were then averaged over the duration of the inhalation.

Emitted Dose (ED) refers to the mass of therapeutic which exits a suitable inhaler device after a firing or dispersion event. The ED is determined using a method based on USP Section 601 Aerosols, Metered-Dose Inhalers and Dry Powder Inhalers, Delivered-Dose Uniformity, Sampling the Delivered Dose from Dry Powder Inhalers, United States Pharmacopeia convention, Rockville, MD, 13th Revision, 222-225, 2007. Contents of capsules are dispersed using either the RS01 HR inhaler at a pressure drop of 4 kPa and a typical flow rate of 60 LPM or the UHR2 RS01 at a pressure drop of 4 kPa and a typical flow rate of 39 LPM. The emitted powder is collected on a filter in a filter holder sampling apparatus. The sampling apparatus is rinsed with a suitable solvent such as water and analyzed using an HPLC method. For gravimetric analysis a shorter length filter holder sampling apparatus is used to reduce deposition in the apparatus and the filter is weighed before and after to determine the mass of powder delivered from the DPI to the filter. The emitted dose of therapeutic is then calculated based on the content of therapeutic in the delivered powder. Emitted dose can be reported as the mass of therapeutic delivered from the DPI or as a percentage of the filled dose.

Thermogravimetric Analysis: Thermogravimetric analysis (TGA) was performed using either the Q500 model or the Discovery model thermogravimetric analyzer (TA Instruments, New Castle, DE). The samples were either placed into an open aluminum DSC pan or a sealed aluminum DSC pan that was then automatically punched open prior to the time of test. Tare weights were previously recorded by the instrument. The following method was employed: Ramp 5.00° C./min from ambient (˜35° C.) to 200° C. The weight loss was reported as a function of temperature up to 140° C. TGA allows for the calculation of the content of volatile compounds within the dry powder. When utilizing processes with water alone, or water in conjunction with volatile solvents, the weight loss via TGA is a good estimate of water content.

X-Ray Powder Diffraction: The crystalline character of the formulations was assessed via powder X-ray diffraction (PXRD). A 20-30 mg sample of material is analyzed in a powder X-ray diffractometer (D8 Discover with LINXEYE detector; Bruker Corporation, Billerica, MA or equivalent) using a Cu X-ray tube with 1.5418A at a data accumulation time 1.2 second/step over a scan range of 5 to 45°2θ and a step size of 0.02°2θ.

DHE Mesylate Content using HPLC. A high-performance liquid chromatography (HPLC) method utilizing a reverse phase C18 column coupled to an ultraviolet (UV) detector has been developed for the bulk content analysis of DHE mesylate formulations. The reverse phase column is equilibrated to 30° C. and the autosampler is set to 5° C. Mobile phase A (3g/L 1-heptanesulfonic acid sodium salt monohydrate in water pH 2.0) and mobile phase B (80% acetonitrile/20% mobile phase A) are used in a gradient elution from a ratio of 60:40 (A:B) to 48:52 (A:B), over the course of a 20 minute run time. Detection is by UV at 220 nm and the injection volume is 5 μL. DHE Mesylate content in powders is quantified relative to a standard curve.

DHE Mesylate Content using UPLC. An ultra-performance liquid chromatography (UPLC) method utilizing a reverse phase C18 column coupled to an ultraviolet (UV) detector has been developed for the bulk content analysis of DHE mesylate formulations. The reverse phase column is equilibrated to 30° C. and the autosampler is set to 5° C. Mobile phase A (water adjusted to pH 2.0 with phosphoric acid) and mobile phase B (80% acetonitrile/20% mobile phase A) are used in a gradient elution from a ratio of 65:35 (A:B) to 47:53 (A:B), over the course of a 12 minute run time. Detection is by UV at 220 nm and the injection volume is 2 μL. DHE Mesylate content in powders is quantified relative to a standard curve.

Particle Size Reduction. The particle size distribution of the crystalline active agent can be modulated using a number of techniques familiar to those of skill in the art, including but not limited to, high-pressure homogenization, high-shear homogenization, jet-milling, pin milling, microfluidization, or wet milling (also known as ball milling, pearl milling or bead milling). Wet milling is often preferred, as it is able to achieve a wide range of particle size distributions, including those in the nanometer (1 μm) size domain.

Particle Size Reduction using Low Energy Wet Milling. One technique for reducing the particle size of the active agent was via low energy wet milling, (also known as roller milling, or jar milling). Suspensions of the active agent were prepared in an anti-solvent, which can be water, or any solvent in which the active agent is not appreciably soluble. Stabilizers, which can be, but are not limited to, non-ionic surfactants or amphiphilic polymers, are then added to the suspension along with milling media, which can be, but are not limited to, spherical with high wear resistance and in the size range from 0.03 to 0.70 millimeters in diameter. The vessels containing the suspensions are then rotated using a jar mill (US Stoneware, East Palestine, OH USA) while taking samples periodically to assess particle size (LA-950, HORIBA, Kyoto, Japan). When the particle size is sufficiently reduced, or when a particle size minimum is reached, the suspension is strained through a sieve to remove the milling media, and the product recovered.

Particle Size Reduction using High Energy Wet Milling. Another technique for reducing the particle size of the active agent was via high-energy wet milling using a rotor-stator, or agitated media mill. Suspensions of the active agent were prepared in an anti-solvent, which can be water, or any solvent in which the active agent is not appreciably soluble. Stabilizers, which can be, but are not limited to, non-ionic surfactants or amphiphilic polymers, are then added to the suspension along with milling media, which can be, but are not limited to, spherical with high wear resistance and in the size range from 0.03 to 0.70 millimeters in diameter. The suspensions are then charged into the mill, which can be operated in either batch or recirculation mode. The process consists of the suspension and milling media being agitated within the milling chamber, which increases the energy input to the system and accelerates the particle size reduction process. The milling chamber and recirculation vessel are jacketed and actively cooled to avoid temperature increases in the product. The agitation rate and recirculation rate of the suspension are controlled during the process. Samples are taken periodically to assess particle size (LA-950, HORIBA, Kyoto, Japan). When the particle size is sufficiently reduced, or when a particle size minimum is reached, the suspension is discharged from the mill.

Particle Size Reduction using Microfluidization. Another technique for reducing the particle size distribution of the active agent was via Microfluidization. Microfluidizer-based processing is a high-shear wet-processing unit operation utilized for particle size reduction of liquids and solids. The unit can be configured with various interaction chambers, which are cylindrical modules with specific orifice and channel designs through which fluid is passed at high pressures to control shear rates. Product enters the unit via the inlet reservoir and is forced into the fixed-geometry interaction chamber at speeds up to 400 m/sec by a high-pressure pump. It is then effectively cooled, if required, and collected in the output reservoir. The process can be repeated as necessary (e.g. multiple “passes”) to achieve the particle size targets. Particle size of the active agent is monitored periodically via laser diffraction (LA-950, HORIBA, Kyoto, Japan). When the particle size is sufficiently reduced, or when a particle size minimum is reached, the suspension is recovered from the unit.

Particle Size Reduction using Jet Milling. Another technique for reducing the particle size distribution of the active agent was via jet milling. Jet mills utilize fluid energy (compressed air or gas) to grind and classify, in a single chamber with no moving parts. Activated by high pressure air, the particles are accelerated into a high speed rotation in a shallow grinding chamber. As the particles impact on one another their size is reduced. Centrifugal force holds larger particles in the grinding rotation area until they have achieved the desired fine particle size. Centripetal force drags the desired particles towards the static classifier where they are allowed to exit upon achieving the correct particle size. The final particle size is controlled by varying the rate of the feed and propellant pressure.

Liquid Feedstock Preparation for Spray Drying. Spray drying homogenous particles requires that the ingredients of interest be solubilized in solution or suspended in a uniform and stable suspension. The feedstock can utilize water, or a combination of water and other miscible solvents such as alcohols or ketones, as the solvent in the case of solutions, or as the continuous phase in the case of suspensions. Feedstocks of the various formulations were prepared by dissolving the soluble components in the desired solvent(s) followed by dispersing the surfactant-stabilized active agent-containing suspension in the resulting solution while mixing, although the process is not limited to this specific order of operations.

Spray Drying Using Niro Spray Dryer. Dry powders were produced by spray drying utilizing a Niro Mobile Minor spray dryer (GEA Process Engineering Inc., Columbia, MD) with powder collection from a cyclone, a product filter or both. Atomization of the liquid feed was performed using a co-current two-fluid nozzle either from Niro (GEA Process Engineering Inc., Columbia, MD) or a Spraying Systems (Carol Stream, IL) 1/4J two-fluid nozzle with gas cap 67147 and fluid cap 2850SS, although other two-fluid nozzle setups are also possible. In some embodiments, the two-fluid nozzle can be in an internal mixing setup or an external mixing setup. Additional atomization techniques include rotary atomization or a pressure nozzle. The liquid feed was fed using gear pumps (Cole-Parmer Instrument Company, Vernon Hills, IL) directly into the two-fluid nozzle or into a static mixer (Charles Ross & Son Company, Hauppauge, NY) immediately before introduction into the two-fluid nozzle. An additional liquid feed technique includes feeding from a pressurized vessel. Nitrogen or air may be used as the drying gas, provided that moisture in the air is at least partially removed before its use. Pressurized nitrogen or air can be used as the atomization gas feed to the two-fluid nozzle. The drying gas inlet temperature can range from 70° C. to 300° C. and outlet temperature from 30° C. to 120° C. with a liquid feedstock rate of 10 mL/min to 100 mL/min. The gas supplying the two-fluid atomizer can vary depending on nozzle selection and for the Niro co-current two-fluid nozzle can range from 5 kg/hr to 50 kg/hr or for the Spraying Systems 1/4J two-fluid nozzle can range from 30 g/min to 150 g/min. The atomization gas rate can be set to achieve a certain gas to liquid mass ratio, which directly affects the droplet size created. The pressure inside the drying drum can range from +3″ WC to −6″ WC. Spray dried powders can be collected in a container at the outlet of the cyclone, onto a cartridge or baghouse filter, or from both a cyclone and a cartridge or baghouse filter.

Spray Drying Using Büchi Spray Dryer. Dry powders were prepared by spray drying on a Büchi B-290 Mini Spray Dryer (BÜCHI Labortechnik AG, Flawil, Switzerland) with powder collection from either a standard or High-Performance cyclone. The system was run either with air or nitrogen as the drying and atomization gas in open-loop (single pass) mode. When run using air, the system used the Büchi B-296 dehumidifier to ensure stable temperature and humidity of the air used to spray dry. When run using nitrogen, a pressurized source of nitrogen was used. Furthermore, the aspirator of the system was adjusted to maintain the system pressure at −2.0″ water column. Atomization of the liquid feed utilized a Büchi two-fluid nozzle with a 1.5 mm diameter or a Schlick 970-0 atomizer with a 0.5 mm liquid insert (Düsen-Schlick GmbH, Coburg, Germany). Inlet temperature of the process gas can range from 100° C. to 220° C. and outlet temperature from 30° C. to 120° C. with a liquid feedstock flowrate of 3 mL/min to 10 mL/min. The two-fluid atomizing gas ranges 12 to 36 g/min. The aspirator rate ranges from 50% to 100%.

Stability Assessment. The physicochemical stability and aerosol performance of select formulations were assessed at 2-8° C., 25° C./60% RH, and when material quantities permitted, 40° C./75% RH as detailed in the International Conference on Harmonisation (ICH) Q1 guidance. Stability samples were stored in calibrated chambers (Darwin Chambers Company Models PH024 and PH074, St. Louis. MO). Bulk powder samples were weighed into amber glass vials, sealed under 30% RH, and induction-sealed in aluminum pouches (Drishield 3000, 3M, St. Paul, MN) with silica desiccant (2.0 g, Multisorb Technologies, Buffalo, NY). Additionally, to assess the stability of the formulations in capsules, the target mass of powder was weighed into a size 3 HPMC capsule (Capsugel Vcaps) at 30% RH or less. Filled capsules were then aliquoted into high-density polyethylene (HDPE) bottles and induction sealed in aluminum pouches with silica desiccant.

Tap Density. Tap density was measured using a modified USP method requiring smaller powder quantities by following USP <616> with the substitution of a 1.5 cc microcentrifuge tube (Eppendorf AG, Hamburg, Germany) or a 0.3 cc section of a disposable serological polystyrene micropipette (Grenier Bio-One, Monroe, NC) with polyethylene caps (Kimble Chase, Vineland, NJ) to cap both ends and hold the powder within the pipette section. Instruments for measuring tap density, known to those skilled in the art, include but are not limited to the Dual Platform Microprocessor Controlled Tap Density Tester (Vankel, Cary, NC) or a SOTAX Tap Density Tester model TD1 (Horsham, PA). Tap density is a standard measure of the envelope mass density. The envelope mass density of an isotropic particle is defined as the mass of the particle divided by the minimum spherical envelope volume within which it can be enclosed.

Bulk Density. Bulk density was estimated prior to tap density measurement by dividing the weight of the powder by the volume of the powder, as estimated using the volumetric measuring device.

Capsule Emitted Powder Mass. A measure of the emission properties of the powders was determined analytically or gravimetrically by using the information obtained from the aPSD tests or emitted geometric diameter by Spraytec. For analytic determination of CEPM, the quantity of DHE remaining in the capsule post-emission was assessed by dissolving the entire capsule in a known quantity of solvent and analytically determining the capsule retention, then subtracting this value from the nominal dose. For gravimetric determination of CEPM, the filled capsule weight was recorded at the beginning of the run and the final capsule weight was recorded after the completion of the run. The difference in weight represented the amount of powder emitted from the capsule (CEPM or capsule emitted powder mass). The CEPM was reported as a mass of powder or as a percent by dividing the amount of powder emitted from the capsule by the total initial particle mass in the capsule.

### EXAMPLE 1

### Dry Powder Formulations of Amorphous DHE

### A. Powder Preparation.

Feedstock solutions were prepared and used to manufacture dry powders composed of amorphous DHE mesylate, a cationic salt and various other excipients. Drug loads from 1.5% to 10% DHE mesylate (amorphous), on a dry basis, were targeted. The feedstock solutions that were used to spray dry particles were made as follows. The required quantity of water and ethanol was weighed into a suitably sized vessel. The API and excipients were added to the solvents and the solution allowed to stir until visually clear. The feedstocks were then spray-dried. Feedstocks were stirred while spray dried. Table 2 lists the components of the feedstocks used in preparation of the dry powders.

TABLE 2
Feedstock Compositions of Formulations I, II and V-XVI

| | | | DHE | | | | Total |
|---|---|---|---|---|---|---|---|
| | Water | Ethanol | Mesylate | NaCl | Mannitol | Leucine | Mass |
| Formulation | (g) | (g) | (g) | (g) | (g) | (g) | (g) |
| I | 10290 | 4410.0 | 15.00 | 228.0 | N/A | 57.00 | 15000 |
| II | 8190.0 | 3510.0 | 15.00 | 142.50 | 142.50 | NA | 12000 |
| V | 2182.5 | 2182.5 | 13.50 | 12.15 | 85.05 | 24.30 | 4500 |
| VI | 194.0 | 194.0 | 0.18 | 9.46 | N/A | 2.36 | 400.0 |
| VII | 194.0 | 194.0 | 0.18 | 5.91 | 5.91 | N/A | 400.0 |
| VIII | 145.5 | 145.5 | 0.90 | 6.48 | N/A | 1.62 | 300.0 |
| IX | 145.5 | 145.5 | 0.90 | 4.05 | 4.05 | NA | 300.0 |
| X | 194.0 | 194.0 | 0.18 | 4.73 | 4.73 | 2.36 | 400.0 |
| XI | 145.5 | 145.5 | 0.90 | 3.24 | 3.24 | 1.62 | 300.0 |
| XII | 194.0 | 194.0 | 0.18 | 8.27 | 1.18 | 2.36 | 400.0 |
| XIII | 194.0 | 194.0 | 0.18 | 1.18 | 8.27 | 2.36 | 400.0 |
| XIV | 194.0 | 194.0 | 0.69 | 4.52 | 4.52 | 2.26 | 400.0 |
| XV | 145.5 | 145.5 | 0.90 | 5.67 | 0.81 | 1.62 | 300.0 |
| XVI | 145.5 | 145.5 | 0.90 | 0.81 | 5.67 | 1.62 | 300.0 |

Dry powders of Formulations I, II, and V were manufactured from these feedstocks by spray drying on the Niro Mobile Minor (GEA Niro, Copenhagen Denmark) with bag filter powder collection. The system was run in open-loop (single pass) mode using nitrogen as the drying and atomization gas. Atomization of the liquid feed utilized a Niro two-fluid nozzle with 5.0 mm cap and 1.0 mm liquid tip. The blower of the system was adjusted to maintain the system pressure at −2.0″ water column.

The following spray drying conditions were followed to manufacture the dry powders on the Niro Mobile Minor. For Formulations I and II the liquid feedstock solids concentration was 3.0 wt %, the process gas inlet temperature was about 165° C. to about 180° C., the process gas outlet temperature was 70° C., the drying gas flowrate was 80.0 kg/hr, the atomization gas flowrate was 250 g/min, and the liquid feedstock flowrate was 50.0 mL/min. For Formulation V the liquid feedstock solids concentration was 3.0 wt %, the process gas inlet temperature was about 155° C. to about 170° C., the process gas outlet temperature was 70° C., the drying gas flowrate was 80.0 kg/hr, the atomization gas flowrate was 175 g/min, and the liquid feedstock flowrate was 50.0 mL/min. The resulting dry powder formulations are reported in Table 3.

Dry powders of Formulations VI-XVI were manufactured from these feedstocks by spray drying on the Büchi B-290 Mini Spray Dryer (BÜCHI Labortechnik AG, Flawil, Switzerland) with cyclone powder collection. The system was run in open-loop (single pass) mode using nitrogen as the drying and atomization gas. Atomization of the liquid feed utilized a Schlick 970-1 nozzle. The aspirator of the system was adjusted to maintain the system pressure at −2.0″ water column.

The following spray drying conditions were followed to manufacture the dry powders on the Büchi B-290. The liquid feedstock solids concentration was 3.0 wt %, the process gas inlet temperature was about 140° C. to 165° C., the process gas outlet temperature was 70° C., the drying gas flowrate was 18.0 kg/hr, the atomization gas flowrate was 30 g/min, and the liquid feedstock flowrate was 6.0 mL/min. The resulting dry powder formulations are reported in Table 2.

TABLE 3
Amorphous DHE dry powder compositions, dry basis

| Formulation | Dry Powder Composition (w/w), dry basis |
|---|---|
| I | 5.0% DHE mesylate, 76.0% sodium chloride, 19.0% leucine |
| II | 5.0% DHE mesylate, 47.5% sodium chloride, 47.5% mannitol |
| V | 10.0% DHE mesylate, 9.0% sodium chloride, 63.0% mannitol, 18.0% leucine |
| VI | 1.5% DHE mesylate, 78.8% sodium chloride, 19.7% leucine |
| VII | 1.5% DHE mesylate, 49.25% sodium chloride, 49.25% mannitol |
| VIII | 10.0% DHE mesylate, 72.0% sodium chloride, 18.0% leucine |
| IX | 10.0% DHE mesylate, 45.0% sodium chloride, 45.0% mannitol |
| X | 1.5% DHE mesylate, 39.4% sodium chloride, 39.4% mannitol, 19.7% leucine |
| XI | 10.0% DHE mesylate, 36.0% sodium chloride, 36.0% mannitol, 18.0% |
| | leucine |
| XII | 1.5% DHE mesylate, 68.95% sodium chloride, 9.85% mannitol, 19.7% |
| | leucine |
| XIII | 1.5% DHE mesylate, 9.85% sodium chloride, 68.95% mannitol, 19.7% |
| | leucine |
| XIV | 5.75% DHE mesylate, 37.7% sodium chloride, 37.7% mannitol, 18.85% |
| | leucine |
| XV | 10.0% DHE mesylate, 63.0% sodium chloride, 9.0% mannitol, 18.0% leucine |
| XVI | 10.0% DHE mesylate, 9.0% sodium chloride, 63.0% mannitol, 18.0% leucine |

### B. Powder Characterization.

The bulk particle size characteristics for the formulations are found in Table 4. The 1 bar/4 bar dispersibility ratio of <1.5 indicates that all formulations are relatively independent of dispersion energy, a desirable characteristic which allows similar particle dispersion across a range of dispersion energies.

TABLE 4
Bulk particle size

| | 0.5 bar | 1 bar | 4 bar | 1 bar:4 bar |
|---|---|---|---|---|
| | Dv[50] ± SD | Dv[50] ± SD | Dv[50] ± SD | Dv[50] |
| Formulation | (μm) | (μm) | (μm) | ratio |
| I | 1.93 ± 0.02 | 1.73 ± 0.03 | 1.40 ± 0.01 | 1.24 |
| II | 2.45 ± 0.02 | 1.98 ± 0.03 | 1.71 ± 0.04 | 1.16 |
| V | 1.87 ± 0.01 | 1.78 ± 0.01 | 1.71 ± 0.01 | 1.04 |
| VI | 2.01 ± 0.01 | 1.87 ± 0.01 | 1.53 ± 0.03 | 1.22 |
| VII | 2.12 ± 0.04 | 1.93 ± 0.02 | 1.80 ± 0.01 | 1.07 |
| VIII | 1.98 ± 0.01 | 1.93 ± 0.01 | 1.71 ± 0.06 | 1.13 |
| IX | 2.16 ± 0.01 | 2.03 ± 0.01 | 1.86 ± 0.0 | 1.09 |
| X | 2.11 ± 0.01 | 2.06 ± 0.01 | 1.93 ± 0.0 | 1.07 |
| XI | 2.15 ± 0.01 | 2.10 ± 0.01 | 1.94 ± 0.03 | 1.09 |
| XII | 2.20 ± 0.01 | 2.08 ± 0.01 | 1.75 ± 0.01 | 1.19 |
| XIII | 2.14 ± 0.03 | 2.08 ± 0.01 | 1.97 ± 0.01 | 1.06 |
| XIV | 2.47 ± 0.01 | 2.36 ± 0.03 | 2.12 ± 0.02 | 1.11 |
| XV | 2.08 ± 0.01 | 2.03 ± 0.01 | 1.88 ± 0.05 | 1.08 |
| XVI | 2.21 ± 0.01 | 2.11 ± 0.02 | 1.94 ± 0.01 | 1.09 |

The aerodynamic particle size and fine particle doses for Formulations I, II and V-XVI are reported in Table 5. The MMAD values of <5 μm and high fine particle dose values (relative to nominal dose) for all formulations indicate a high proportion of the dose would be expected to deposit in the central and conducting airways, indicating these formulations are suitable for inhalation.

TABLE 5
Aerodynamic particle size for Formulations I, II, and V-XVI.

| | MMAD ± SD | FPD < 5 μm ± SD | CEPM ± SD |
|---|---|---|---|
| Formulation | (μm) | (% nominal dose) | (% nominal dose) |
| I | 2.29 ± 0.02 | 80.25% ± 1.86% | 95.88% ± 0.41% |
| II | 2.73 ± 0.02 | 61.19% ± 0.36% | 98.76% ± 0.19% |
| V | 2.44 ± 0.03 | 76.20% ± 2.30% | 98.37% ± 0.21% |
| VI | 2.38 ± 0.02 | 75.65% ± 2.56% | 99.27% ± 0.13% |
| VII | 2.90 ± 0.09 | 52.05% ± 0.32% | 98.49% ± 0.28% |
| VIII | 2.39 ± 0.04 | 75.66% ± 3.12% | 97.95% ± 0.19% |
| IX | 2.77 ± 0.05 | 56.88% ± 1.46% | 95.67% ± 0.29% |
| X | 2.19 ± 0.01 | 79.71% ± 8.28% | 99.23% ± 0.67% |
| XI | 2.03 ± 0.07 | 72.30% ± 11.64% | 98.31% ± 1.37% |
| XII | 2.23 ± 0.01 | 80.48% ± 2.10% | 97.63% ± 2.14% |
| XIII | 2.52 ± 0.00 | 77.86% ± 2.16% | 98.89% ± 0.20% |
| XIV | 2.34 ± 0.04 | 73.83% ± 1.57% | 97.91% ± 0.27% |
| XV | 2.18 ± 0.07 | 79.17% ± 1.50% | 97.92% ± 0.47% |
| XVI | 2.40 ± 0.02 | 72.87% ± 2.99% | 97.62% ± 0.14% |

The weight loss of Formulations I, II and V-XIV were measured via TGA and are reported in Table 6.

TABLE 6
Weight Loss via TGA of Formulations I, II, and V-XVI

| Formulation | Weight loss via TGA (%) |
|---|---|
| I | 0.858 |
| II | 0.775 |
| V | 0.608 |
| VI | 0.426 |
| VII | 0.693 |
| VIII | 0.710 |
| IX | 0.702 |
| X | 0.479 |
| XI | 0.595 |
| XII | 0.402 |
| XIII | 0.539 |
| XIV | 0.499 |
| XV | 0.628 |
| XVI | 0.624 |

The DHE mesylate content of Formulations I, II and V-XIV is presented in Table 7.

TABLE 7
DHE Content of Formulations I, II and V-XIV

| | Bulk content |
|---|---|
| Formulation | (% DHE w/w) |
| I | 5.14 |
| II | 5.03 |
| V | 10.05 |
| VI | 1.46 |
| VII | 1.47 |
| VIII | 9.95 |
| IX | 10.14 |
| X | 1.41 |
| XI | 10.08 |
| XII | 1.48 |
| XIII | 1.46 |
| XIV | 5.77 |
| XV | 10.13 |
| XVI | 10.01 |

The bulk density values for Formulations I, II and V were assessed and found to be 0.21 g/cc, 0.10 g/cc and 0.32 g/cc, respectively.

The tapped density values for Formulations I, II and V were assessed and found to be 0.40 g/cc, 0.26 g/cc and 0.73 g/cc, respectively.

### EXAMPLE 2

### Dry Powder Formulations of Nanocrystalline DHE

### A. Powder Preparation.

The nanocrystalline DHE mesylate was prepared by compounding 50.0 g of DHE mesylate (Olon, lot #18009GR40S) in 440.0 g of water with 5.00 g of sodium sulfate (Millipore, lot # F2099145 019) and 5.01 g of polysorbate 80 (Sigma-Aldrich, lot # BCCB9820). Once all of the DHE mesylate was suspended, the formulation was processed on a Netzsch MiniCer using 560.3 g of 0.2 mm yttria-stabilized zirconia grinding media (Netzsch, lot # 2006356). The mill speed was set at 3000 rpm, the suspension pump speed was set at 216 rpm, and the chiller temperature was 5° C. After start-up, the mill speed was reduced to 2000 rpm and the suspension pump speed was reduced to 100 rpm. The total run time was 35 minutes. The final median particle size (Dv(50)) of the milled suspension was 271 nm.

The jet-milled DHE mesylate was prepared by feeding 27.54 g of DHE mesylate (Olon lot # 18009GR40S) into a jet mill (Sturtevant 2 inch Qualification Micronizer) at a feeder setting of 4.5 (corresponding to an approximate feed rate of 1 g/min). The feed pressure of the mill was set to 70 psig, and the grinding pressure was set to 45 psig. The final median particle size (Dv(50)) of the jet-milled DHE mesylate was 1.52 μm.

Feedstock solutions were prepared and used to manufacture dry powders composed of crystalline DHE mesylate, polysorbate 80, sodium sulfate and other additional excipients. Drug loads of 10 wt % DHE mesylate, on a dry basis, were targeted. Formulation III utilized nanocrystalline DHE mesylate and Formulation IV utilized microcrystalline (via jet-milling) DHE mesylate. The feedstock solutions that were used to spray dry particles were made as follows. The required quantity of water was weighed into a suitably sized vessel. The excipients were added to the water and the solution allowed to stir until visually clear. The DHE-containing suspension, or crystalline DHE, was then added to the excipient solution and stirred until visually homogenous. The feedstocks were then spray-dried. Feedstocks were stirred while spray dried. Table 8 lists the components of the feedstocks used in preparation of the dry powders.

TABLE 8
Feedstock compositions for Formulations III and IV

| | | DHE | Polysor- | Sodium | | Total |
|---|---|---|---|---|---|---|
| Formu- | Water | Mesylate | bate 80 | Sulfate | Mannitol | mass |
| lation | (g) | (g) | (g) | (g) | (g) | (gm) |
| III | 6050.89 | 18.15 | 1.82 | 80.78 | 80.78 | 6050.9 |
| IV | 5820.0 | 18.00 | 1.80 | 80.10 | 80.1 | 6000.0 |

Dry powders of Formulations III and IV were manufactured from these feedstocks by spray drying on the Niro Mobile Minor (GEA Niro, Copenhagen Denmark) with bag filter powder collection. The system was run in open-loop (single pass) mode using nitrogen as the drying and atomization gas. Atomization of the liquid feed utilized a Niro two-fluid nozzle with 5.0 mm cap and 1.0 mm liquid tip. The blower of the system was adjusted to maintain the system pressure at −2.0″ water column.

The following spray drying conditions were followed to manufacture the dry powders. For Formulations III and IV, the liquid feedstock solids concentration was 3.0%, the process gas inlet temperature was about 176° C. to 183° C., the process gas outlet temperature was 65° C., the drying gas flowrate was 80.0 kg/hr, the atomization gas flowrate was 365 g/min, and the liquid feedstock flowrate was 50.0 mL/min. The resulting dry powder formulations are reported in Table 9.

TABLE 9
Crystalline DHE dry powder compositions, dry basis

| Formulation | Dry Powder Composition (w/w), dry basis |
|---|---|
| III | 10.0% DHE mesylate (nanocrystalline), 44.5% sodium |
| | sulfate, 44.5% mannitol, 1.0% polysorbate 80 |
| IV | 10.0% DHE mesylate (microcrystalline), 44.5% sodium |
| | sulfate, 44.5% mannitol, 1.0% polysorbate 80 |

### B. Powder Characterization.

The bulk particle size characteristics for the formulations are found in Table 10. The 1 bar/4 bar dispersibility ratio of <1.5, indicate that all formulations are relatively independent of dispersion energy, a desirable characteristic which allows similar particle dispersion across a range of dispersion energies.

TABLE 10
Bulk particle size for Formulations III and IV

| | 0.5 bar | 1 bar | 4 bar | 1 bar:4 bar |
|---|---|---|---|---|
| | Dv[50] | Dv[50] | Dv[50] | Dv[50] |
| Formulation | (μm) | (μm) | (μm) | ratio |
| III | 2.08 ± 0.02 | 1.79 ± 0.02 | 1.63 ± 0.01 | 1.10 |
| IV | 2.15 ± 0.04 | 1.89 ± 0.03 | 1.71 ± 0.02 | 1.11 |

The aerodynamic particle size data for Formulations III and IV are presented in Table 11. The MMAD values of <5 μm and high fine particle dose values (relative to nominal dose) for all formulations indicate a high proportion of the dose would be expected to deposit in the central and conducting airways, indicating these formulations are suitable for inhalation.

TABLE 11
Aerodynamic particle size data for Formulations III and IV

| | MMAD | FPD < 5 μm | CEPM |
|---|---|---|---|
| Formulation | (μm) | (% nominal dose) | (%) |
| III | 3.04 ± 0.02 | 55.10% ± 0.99% | 95.61% ± 1.23% |
| IV | 3.01 ± 0.05 | 54.16% ± 1.99% | 98.62% ± 0.18% |

The weight loss of Formulations III and IV were measured via TGA and were found to be 1.073% and 0.995% respectively.

The DHE mesylate content of Formulations III and IV were measured via UPLC and found to be 10.10% and 9.80%, respectively.

The bulk density values of Formulations III and IV were assessed and found to be 0.21 g/cc and 0.24 g/cc, respectively.

The tapped density values of Formulations III and IV were assessed and found to be 0.52 g/cc and 0.54 g/cc, respectively.

### EXAMPLE 3

### Pharmacokinetic Profiles of DHE with Dry Powder Formulations I-IV

The pharmacokinetic profiles of DHE following administration of Formulations I-IV in a dog model were assessed with the following protocol. A single dose inhalation of the dry powder formulation was administered to male Beagle dogs for 30 minutes, at target doses of 250, 400 and 600 μg/kg, on days 1, 6, and 12, according to Table 12.

TABLE 12
Group designation and dose level for pharmacokinetic study.

| | | | Target Total | Target API | |
|---|---|---|---|---|---|
| | | Treatment Day | Delivered API | Aerosol | |
| Group | Formulation | (Group | Dose Level | Concentration | No. of |
| No. | No. | Designation) | (μg/kg) | (μg/L)^{a} | Males |
| 1 | III^{b} | 1 (Low Dose) | 250 | 19.3 | 3 |
| | | 6 (Mid Dose) | 400 | 30.9 | |
| | | 12 (High Dose) | 600 | 46.3 | |
| 2 | IV^{b} | 1 (Low Dose) | 250 | 19.3 | 3 |
| | | 6 (Mid Dose) | 400 | 30.9 | |
| | | 12 (High Dose) | 600 | 46.3 | |
| 3 | II^{c} | 1 (Low Dose) | 250 | 19.3 | 3 |
| | | 6 (Mid Dose) | 400 | 30.9 | |
| | | 12 (High Dose) | 600 | 46.3 | |
| 4 | I^{c} | 1 (Low Dose) | 250 | 19.3 | 3 |
| | | 6 (Mid Dose) | 400 | 30.9 | |
| | | 12 (High Dose) | 600 | 46.3 | |

^{a}Target aerosol concentrations were calculated based on an estimated body weight of 10 kg;
^{b}Target dose levels and aerosol concentrations were listed in terms of the percentage of dihydroergotamine (DHE) which was at 10% (w/w) in the specific test item formulations;
^{c}Target dose levels and aerosol concentrations were listed in terms of the percentage of dihydroergotamine (DHE) which was at 5% (w/w) in the specific test item formulations.

At least 96 hours of an observation/washout period was allowed for all animals before the start of dosing at the higher dose level between the scheduled treatments. A series of 10 blood samples (approximately 1 mL each) were collected from each dog at Pre-dose, 0.5 (±2 minutes), 1, 1.25, 1.5, 2.5, 4.5, 8.5, 12.5 and 24.5 hours from the start of the exposure on Days 1, 6 and 12. For this purpose, each dog was bled by venipuncture and the samples were collected into tubes containing the anticoagulant, K₃EDTA. Tubes were placed on wet ice pending processing. Following collection, blood samples for plasma were processed within 2 hours of collection. Samples were centrifuged (1000 g for 10 minutes at approximately 4° C.) and the resulting plasma was aliquoted into two aliquots and stored frozen (≤−60° C.) prior to bioanalysis.

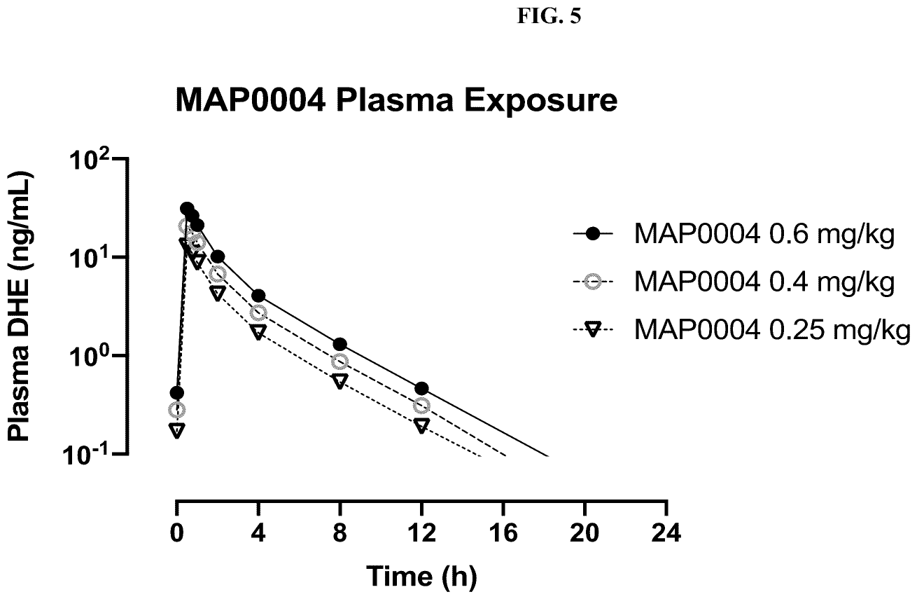

The concentration-over-time curves associated with the measurements obtained in this pharmacokinetic study are provided in FIGS. 1-4. The pharmacokinetic parameters obtained for each dose level of Formulations I-IV are also tabulated in Table 13. For purposes of comparison, an analogous set of pharmacokinetic data for MAP0004 was modelled based on published pharmacokinetic data obtained in a similar dog model (Armer, T. A., et al. Toxicologic Pathology (2011) 39(3): 544-552). The modeled plasma concentration over time curve for MAP0004 is shown in FIG. 5, and the modeled pharmacokinetic parameters for MAP0004 are included in Table 13.

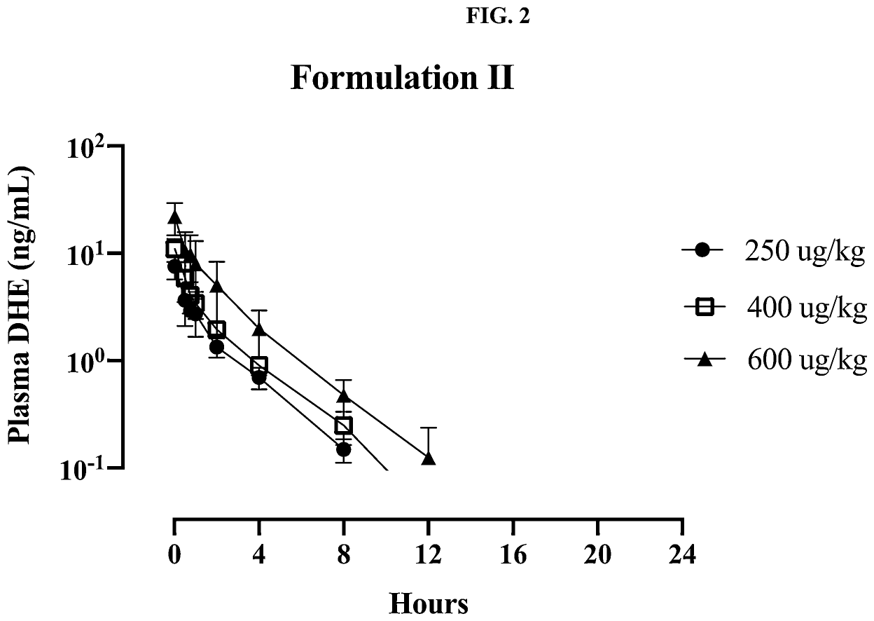

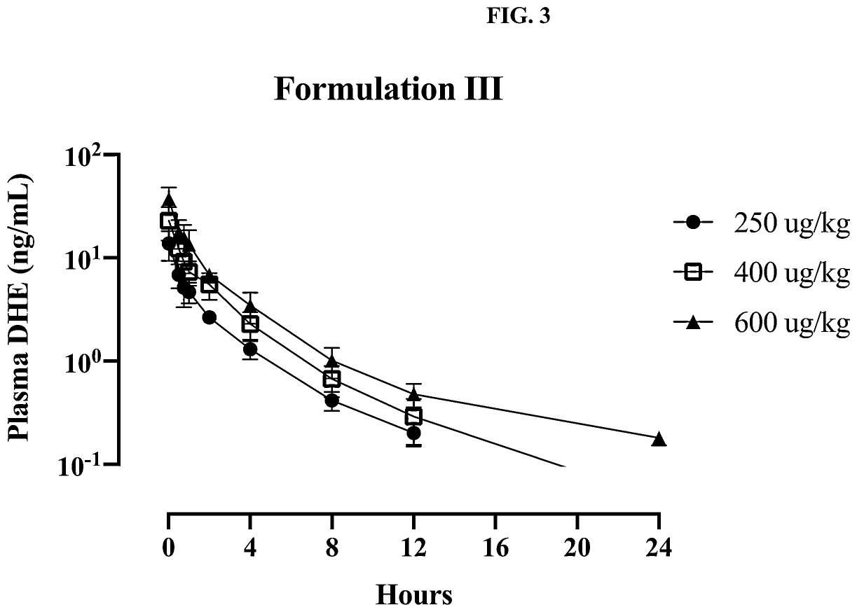

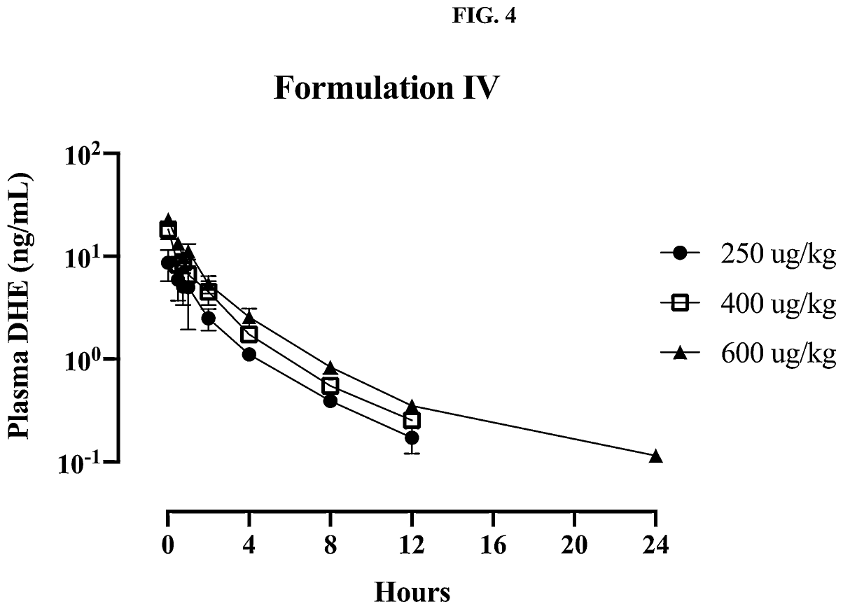

Administration of the amorphous DHE Formulations I and II led to relatively shorter t_{1/2} at each dose level, compared to the crystalline DHE Formulations III and IV, particularly at the highest target dose level. This trend is apparent by observing the difference in the dose concentration curves shown in FIGS. 1 and 2 (amorphous DHE dry powders) relative to FIGS. 3 and 4 (crystalline DHE dry powders). Similarly, the amorphous DHE-containing dry powders resulted in lower AUC (e.g., AUCₗₐₛₜ) relative to the crystalline DHE-containing dry powder formulations. These data suggest that administering the amorphous DHE-containing dry powder formulations can achieve relatively the same Cₘₐₓ and Tₘₐₓ as crystalline DHE-containing dry powder formulations, for an equivalent therapeutic effect, while reducing drug exposure, which is expected to lower incidence and/or reduce severity of side-effects.

Surprisingly, it was discovered that the pharmacokinetic profiles obtained in dogs following administration via inhalation of dry powder Formulations I-IV were substantially similar to the modelled pharmacokinetic data obtained for inhaled MAP0004 in dogs. Thus, it is expected that the presently disclosed dry powder formulations will have substantially the same pharmacokinetic profile as MAP0004 when administered to humans via inhalation (see, e.g., Shrewsbury, S. B. et al. Headache (2008) 48:355-367 for a human pharmacokinetic study of MAP0004 via inhalation). Further details on modeling data to predict pharmacokinetic parameters is provided in Example 5.

TABLE 13
Pharmacokinetic Parameters of Dihydroergotamine in Dogs for Formulations I-IV and MAP0004

| | Dose | t_{1/2} | Cₘₐₓ | AUCₗₐₛₜ | ^{a}DN | ^{b}DN | ^{b}DN | ^{b}DN | ^{b}DN |
|---|---|---|---|---|---|---|---|---|---|
| F | (μg/kg) | (h) | (ng/mL) | (ng · h/mL) | Cₘₐₓ | AUCₗₐₛₜ | AUC_{0-24 h} | AUC_{0-8 h} | AUC_{0-4 h} |
| MAP0004* | 250 | NA | 11 | 28 | 0.044 | 0.11 | 0.11 | 0.104 | 0.084 |
| | 400 | NA | 21 | 45 | 0.053 | 0.11 | 0.11 | 0.103 | 0.085 |
| | 600 | NA | 31 | 68 | 0.052 | 0.11 | 0.11 | 0.103 | 0.085 |
| I | 211 | 1.75 | 11.5 | 16.6 | 0.055 | 0.08 | 0.07 | 0.078 | 0.067 |
| | 414 | 1.76 | 18.9 | 29.0 | 0.046 | 0.07 | 0.06 | 0.068 | 0.057 |
| | 555 | 2.01 | 22.1 | 41.4 | 0.040 | 0.07 | 0.07 | 0.072 | 0.061 |
| II | 231 | 1.88 | 7.58 | 12.0 | 0.030 | 0.05 | 0.04 | 0.052 | 0.043 |
| | 463 | 2.15 | 11.0 | 17.3 | 0.020 | 0.04 | 0.03 | 0.036 | 0.030 |
| | 743 | 1.96 | 22.0 | 37.9 | 0.030 | 0.05 | 0.04 | 0.049 | 0.041 |
| III | 283 | 2.75 | 13.8 | 23.6 | 0.050 | 0.08 | 0.07 | 0.078 | 0.064 |
| | 453 | 3.62 | 22.9 | 42.5 | 0.050 | 0.09 | 0.08 | 0.086 | 0.071 |
| | 698 | 7.00 | 37.1 | 67.1 | 0.050 | 0.10 | 0.08 | 0.085 | 0.071 |
| IV | 258 | 2.96 | 8.62 | 19.9 | 0.033 | 0.08 | 0.07 | 0.072 | 0.059 |
| | 459 | 2.38 | 18.2 | 33.2 | 0.040 | 0.07 | 0.07 | 0.068 | 0.057 |
| | 679 | 5.69 | 22.7 | 48.4 | 0.033 | 0.07 | 0.06 | 0.063 | 0.052 |

F = formulation number;
*MAP0004 data based on modelling;
^{a}dose-normalized (DN) Cₘₐₓ is expressed as (ng/mL)/(μg/kg);
^{b}all dose-normalized (DN) AUC values are expressed as (ng · h/mL)/(μg/kg).

### EXAMPLE 4

### Toxicology Assay of Dry Powder Formulation V in a Rat Model

Toxicology assay of Formulation V was tested according to the following protocol. 252 Sprague-Dawley rats (126 males, 126 females) were separated into 5 groups to receive 14-consecutive days inhalation (30 minutes/day) of either a control (air or placebo) or Formulation V. Group 1 and 2 were control groups that received air (Group 1) or a placebo (dry powder without DHE; Group 2). Groups 3-5 were the low dose, mid dose, and high dose groups that received Formulation V at an achieved delivered dose level of 299, 438, or 645 μg/kg/day, respectively.

Microscopic pathology was used to examine the nasal cavities, nasopharynx, larynx, and lungs of the rats. All changes were focal and localized, and were absent or reduced after recovery period, and considered non-adverse. No change in the rats body weight, food consumption, ophthalmoscopy, clinical chemistry, hematology, coagulation, or urinalysis were observed. There was no mortality throughout the study. Non-adverse clinical signs in the rats included salivation, wet or stained fur, skin and/or eye discoloration, and piloerection that generally lasted 1 to 2 hours after dosing only. No changes in the organ weights of the rats was observed.

In conclusion, based on the toxicological parameters observed in this study, the No Observed Adverse Effect Level (NOAEL) was considered to be at the achieved total DHE delivered dose level of 645 μg/kg/day (high dose) for the rats when treated with Formulation V for 14 consecutive days by inhalation.

### EXAMPLE 5

### 14-Day Pharmacokinetic and Toxicology Study in a Dog Model

The pharmacokinetics of the respirable DHE dry powder was assessed over a period of fourteen days in a dog model, using the following protocol. Test and placebo control items were administered to Beagle dogs once daily by inhalation, for a period of 30 minutes per day for 14 consecutive days. The study involved 50 Beagle dogs (25 male, 25 female) divided into five groups which included two control groups: Group 1 (air), and Group 2 (placebo of dry powder without DHE mesylate); and three treatment groups: Group 3 (low dose of Formulation V; achieved dose level of 265 μg/kg), Group 4 (mid dose of Formulation V; achieved dose level of 423 μg/kg, and Group 5 (high dose of Formulation V; achieved dose level of 705 μg/kg).

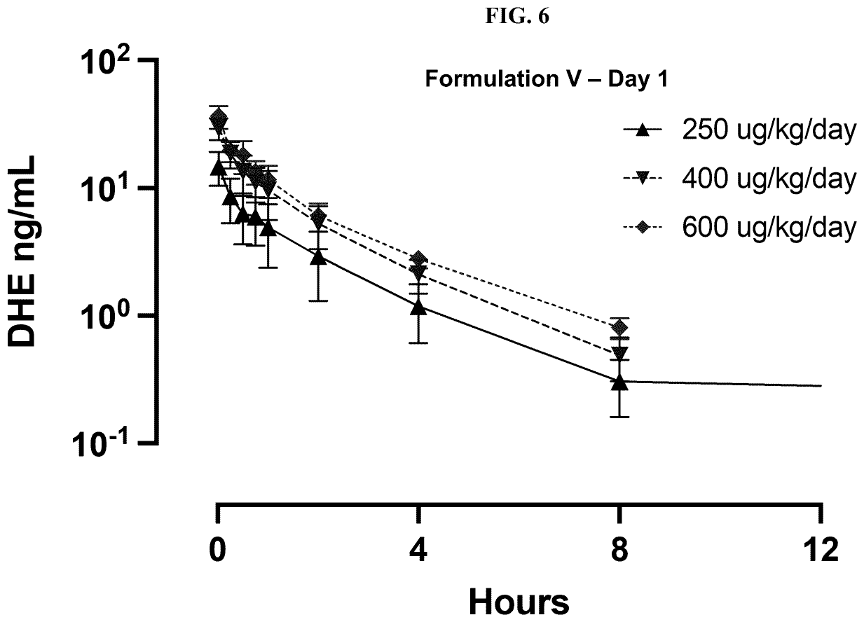

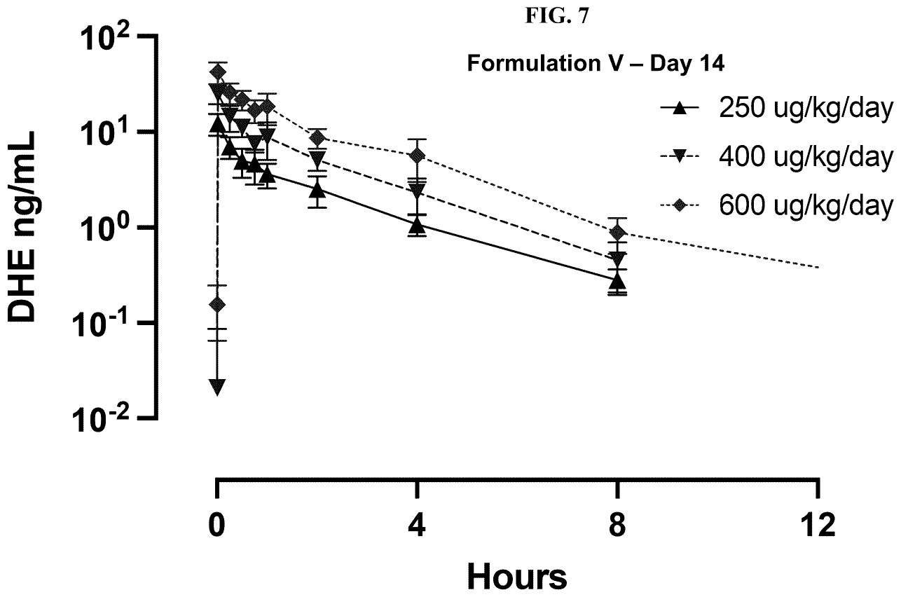

The pharmacokinetic data recorded at Day 1 and Day 14 of the study is provided in Table 14 below, and the corresponding plasma DHE concentration-time curves are provided in FIGS. 6 and 7, respectively. Cₘₐₓ was recorded immediately post exposure on days 1 and 14. On days 1 and 14 rapid clearance of DHE was observed, as indicated by the AUC₀₋₄ₕ at Day 1 and Day 14 being greater than 80% relative to the AUC₀₋₂₄ₕ, the AUC₀₋₈ₕ at Day 1 and Day 14 being greater than 95% relative to the AUC₀₋₂₄ₕ, and with DHE generally below the limit of quantification after 8 hours on both Days 1 and 14. No accumulation was observed in the study, as evidenced by an accumulation ratio (AR) no greater than 1.5 at the highest dose.

TABLE 14
Pharmacokinetic data from 14-day dog study.

| | | Day 1 | | | | | Day 14 | | | | | |
|---|---|---|---|---|---|---|---|---|---|---|---|---|
| | | Cₘₐₓ | t_{1/2} | AUC_{0-4 h} | AUC_{0-8 h} | AUC_{0-INF} | Cₘₐₓ | t_{1/2} | AUC_{0-4 h} | AUC_{0-8 h} | AUC_{0-INF} | AR |
| ADL | Sex | (ng/mL) | (hr) | (ng*h/mL) | (ng*h/mL) | (ng*h/mL) | (ng/mL) | (hr) | (ng*h/mL) | (ng*h/mL) | (ng*h/mL) | (AUC) |
| 265 | M | 15.2 | 2.41 | 21.9 | 25.5 | 27.5 | 13.8 | 1.92 | 18.4 | 21.6 | 22.5 | 0.8 |
| | F | 14.4 | 1.97 | 16.8 | 19.2 | 19.8 | 10.8 | 2.03 | 13.5 | 15.7 | 16.4 | 0.8 |
| 423 | M | 27.6 | 1.82 | 38.9 | 44.1 | 45.5 | 25.5 | 1.83 | 34.8 | 41.2 | 42.7 | 0.9 |
| | F | 31.2 | 1.72 | 36.5 | 41.7 | 42.9 | 25.0 | 1.68 | 31.6 | 36.2 | 37.2 | 0.9 |
| 705 | M | 33.1 | 2.05 | 46.4 | 53.9 | 56.3 | 43.9 | 1.67 | 67.0 | 80.8 | 83.1 | 1.5 |
| | F | 39.9 | 2.11 | 44.8 | 51.7 | 54.2 | 41.0 | 2.69 | 57.8 | 70.2 | 76.4 | 1.4 |

ADL = Achieved Dose Level (μg/kg); AR = accumulation ratio (AUC) (Day 14/Day 1).

No change in body weight, food consumption, ophthalmoscopy, clinical chemistry, hematology, coagulation, or urinalysis was observed in the dogs. Also, no effects on respiratory or electrocardiogram parameters of the dogs were observed. Clinical signs were limited to salivation, retching, emesis, and tremors, which were observed only during dosing and for a short period after, that was fully resolved by the time animals were checked in the evening. Emesis in the dogs tended to abate over the course of the 14 day dosing period. These effects were considered dose limiting but were not considered adverse, and doses were not increased primarily for animal welfare reasons to avoid dogs vomiting into the respiration apparatus used for the study, that could result in potential aspiration. No changes in organ weights or any microscopic pathology findings were observed at any dose administered to the dogs. The no observed adverse effect level (NOAEL) was 705 μg/kg/day.

The pharmacokinetic data was then compared with a modelled set of data obtained from published pharmacokinetics of MAP0004 inhaled DHE formulation in dogs (Armer et al. Toxicology Pathology (2011) 39(3):544-552) and in humans (Shrewsbury et al. Headache (2008) 48(3):355-367). Comparison was also made to pharmacokinetics of DHE in humans following administration of an intravenous formulation of DHE (D.H.E. 45®), provided in Shrewsbury et al. 2008 (supra). A comparison was made between the data obtained from 14-day PK studies with Formulation V in dogs, and the modeled data of MAP0004 in dogs, which demonstrated comparable AUC and slightly higher Cₘₐₓ with Formulation V.

Without wishing to be bound by any particular theory, it is believed that administration of a respirable dry powder disclosed herein comprising amorphous DHE mesylate (e.g., Formulation V) can achieve a higher Cₘₐₓ, relative to crystalline DHE-based compositions (e.g., MAP0004), as amorphous DHE can dissolve more rapidly in the lung enter the bloodstream more rapidly compared to crystalline DHE formulations. Thus, using the dry powders comprising amorphous DHE disclosed herein, a relatively higher Cₘₐₓ can be achieved compared to other DHE formulations, which can provide more rapid and pronounced relief of migraine, headache, or symptoms in the subject. An additional advantage of the dry powders disclosed herein is that the Tₘₐₓ is rapid (Cₘₐₓ was observed immediately after exposure), and the Cₘₐₓ is within an optimum level to achieve relief of a migraine, headache, or symptom thereof still being relatively low compared to the Cₘₐₓ observed following intravenous DHE administration. This blunting of the Cₘₐₓ using the dry powders disclosed herein can result in much lower incidence of undesirable side effects such as emesis, or completely avoid these side effects, that is commonly experienced with intravenous administration of DHE. A comparison of data collected using Formulation V in dog, compared to MAP0004 and intravenous formulations (DHE 45) is summarized below in Table 15.

TABLE 15
Comparison of Formulation V in dog, MAP0004
dog and human dosing, DHE45 human dosing

| | Total | Delivered | | | | |
|---|---|---|---|---|---|---|
| | delivered | dose | Cₘₐₓ | AUC_{0-iINF} | DN Cₘₐₓ | DN AUC_{0-INF} |
| | dose (mg) | (mg/kg) | (ng/mL) | (ng*h/mL) | (ng/mL)/mg | (ng*h/mL)/mg |
| Formulation | 5.417 | 0.7050 | 36.5 | 55.3 | 6.74 | 10.21 |
| V in Dog | | | | | | |
| MAP0004 | 4.532 | 0.4400 | 22.95 | 46.35 | 5.06 | 10.23 |
| Dog | | | | | | |
| DHE45 | 1.000 | 0.0130 | 45.29 | 10.164 | 45.29 | 10.16 |
| Human⁴ | | | | | | |
| MAP0004 | 1.320 | 0.0171 | 5.241 | 11.686 | 3.97 | 8.85 |
| Human⁴ | | | | | | |
| MAP0004 | 0.880 | 0.0114 | 3.648 | 8.116 | 4.15 | 9.22 |
| Human⁴ | | | | | | |

DN = dose normalized (calculated by exposure/achieved dose). MAP0004 dog data is modeled from Armer et al. 2011 (supra). MAP0004 clinical dose (human) expressed as fine particle dose (FPD) (~44% of nominal) per Shrewsbury et al. 2008 (supra).

### Data Modelling

As the dose normalized data is consistent across formulations in humans and dogs, pharmacokinetic data obtained from administration of DHE in dogs can be used to accurately predict clinical exposure in humans. For example, MAP0004 dog exposure data was used to predict human exposure with good accuracy, which was confirmed by comparison of the predicted human exposure data and published human exposure data of MAP0004 (see, e.g., Shrewsbury et al. (2008)). The predicted clinical exposure was calculated by multiplying the MAP0004 fine particle dose (FPD) (clinical dose) by the dose-normalized dog exposure. The predicted clinical exposure at that FPD was calculated within 15-20% accuracy. A summary of the modeling and scaling used to confirm this approach is provided below in Table 16.

TABLE 16
Comparing predicted clinical exposure (modeled from published pharmacokinetic data of
MAP0004 in dogs) to published clinical exposure of MAP0004 at 1.32 and 0.88 mg doses.

| | | Predicted Clinical | Actual | Predicted Clinical | Actual |
|---|---|---|---|---|---|
| Actual Dog MAP0004 | | Exposure | Clinical Exposure² | Exposure | Clinical Exposure² |
| Dose Normalized Data¹ | | (1.32 mg Dose) | (1.32 mg Dose) | (0.88 mg Dose) | (0.88 mg Dose) |
| DN Cₘₐₓ | 5.06 | 6.68 | 5.24 | 4.46 | 3.65 |
| (ng/mL)/mg | | | | | |
| DN AUC_{0-INF} | 10.23 | 13.5 | 11.69 | 9.00 | 8.12 |
| (ng*h/mL)/mg | | | | | |

¹Data from Armer et al. (2011);
²Data from Shrewsbury et al. (2008).

Based on the ability to model human clinical exposure from data obtained from a dog model, it was possible to model clinical exposure from administering Formulation V. Specifically, data obtained from the pharmacokinetic study of Formulation V in dogs described above was used to predict clinical exposure for Formulation V based on dose normalized exposure with nominal dose and fine particle dose. The predicted clinical exposure is presented in Table 17 below.

TABLE 17
Using PK data in dogs to predict human PK (Cₘₐₓ and
AUC_{0-inf}) of Formulation V (nominal dose and FPD)

| | | Predicted | Predicted | Predicted | Predicted |
|---|---|---|---|---|---|
| Nominal Dose | FPD | Cₘₐₓ | AUC_{0-INF} | Cₘₐₓ | AUC_{0-INF} |
| (mg) of | (mg)¹ of | (ng/mL) | (ng*h/mL) | (ng/mL) | (ng*h/mL) |
| Formulation V | Formulation V | at Nominal Dose | at Nominal Dose | at FPD | at FPD |
| 1.5 | 0.9 | 10.11 | 15.31 | 6.06 | 9.19 |
| 1.0 | 0.6 | 6.74 | 10.21 | 4.04 | 6.13 |
| 0.5 | 0.3 | 3.37 | 5.10 | 2.02 | 3.06 |

Calculations using DN Cₘₐₓ and AUC_{0-INF} for Formulation V in the dog of 6.74 ng/mL and 10.21 ng*h/mL.
¹FPD assumed max of 60% of nominal dose.

Without wishing to be bound by theory, it is believed that the AUC over the first 2 hours is most predictive of efficacy with a target therapeutic window for exposure, in terms of Cₘₐₓ, between 1 ng/mL and 13 ng/mL, and a target AUC_{0-INF} of no more than approximately 12 ng*h/mL. As the data presented here demonstrates, the dry powder formulations disclosed herein can effectively achieve these optimal parameters.

In summary, Formulation V was well tolerated in dogs, and there were no microscopic pathology findings at any dose. Systemic exposure is very rapid (Tₘₐₓ immediately after exposure), with rapid clearance (>80% of the AUC occurs within the first 4 h after dose, and >95% in the first 8 h after dose, fitting well with the reported efficacy determination based on exposure over first 2 hours). Administration of Formulation V in dogs resulted in very similar exposure as reported for MAP0004 administration in dogs, for a given dose delivered. Therefore predictions of human exposure to Formulation V and other dry powder formulations disclosed herein can be determined based on pharmacokinetic data obtained in dogs. These predicted clinical exposures suggest Formulation V will be efficacious and well tolerated in humans at all target doses.

The content of each of the patents, patent applications, patent publications and published articles cited in this specification are herein incorporated by reference in their entirety

1. A dry powder comprising respirable dry particles that comprise dihydroergotamine (DHE) or a salt, hydrate, or polymorph thereof; a monovalent metal cation salt; and one or more excipients.

2. The dry powder of claim 1, wherein the DHE or the salt, hydrate, or polymorph thereof is present in an amount of between about 1% and about 30% by weight of the respirable dry particles.

3. The dry powder of claim 1, wherein the dry powder comprises a first excipient and a second excipient, and the DHE or the salt, hydrate, or polymorph thereof is present in an amount of between about 1% and about 30% by weight; the monovalent metal cation salt is present in an amount of between about 2% and about 25% by weight; the first excipient is present in an amount of between about 35% and about 75% by weight; and the second excipient is present in an amount of between about 12% and about 25% by weight, wherein all percentages are weight percentages on a dry basis and all the components of the respirable dry particles amount to 100%.

4. The dry powder of claim 1, wherein the DHE or the salt, hydrate, or polymorph thereof comprises DHE mesylate.

5. The dry powder of claim 1, wherein the monovalent metal cation salt comprises a sodium salt, a potassium salt, or a lithium salt.

6. The dry powder of claim 1, wherein the one or more excipients comprise a sugar, a sugar alcohol, an oligosaccharide, an amino acid, or a combination thereof.

7. The dry powder of claim 1, wherein the respirable dry particles comprise DHE mesylate, sodium chloride, leucine, and mannitol, wherein the DHE mesylate is present in an amount of between about 1% and about 30% by weight; the sodium chloride is present in an amount of between about 2% and about 25% by weight; the mannitol is present in an amount of between about 35% and about 75% by weight; and the leucine is present in an amount of between about 5% and about 35% by weight; wherein all percentages are weight percentages on a dry basis and all the components of the respirable dry particles amount to 100%.

8. The dry powder of claim 1, wherein the respirable dry particles comprise DHE mesylate, sodium chloride, leucine, and mannitol, wherein the DHE mesylate is present in an amount of about 1% to about 15% by weight; the sodium chloride is present in an amount of about 4% to about 14% by weight; the mannitol is present in an amount of about 55% to about 75% by weight; and the leucine is present in an amount of about 12% to about 25% by weight; wherein all percentages are weight percentages on a dry basis and all the components of the respirable dry particles amount to 100%.

9-18. (canceled)

19. The dry powder of claim 1, wherein the respirable dry particles have:
(i) a volume median geometric diameter (VMGD) of about 10 microns or less;
(ii) a dispersibility ratio (1 bar/4 bar) of less than about 1.5 as measured by laser diffraction (RODOS/HELOS system); or
(iii) a tap density of between about 0.1 g/cc and 1.0 g/cc.

20. (Canceled)

21. The dry powder of claim 1, wherein the dry powder has:
(i) a Fine Particle Fraction (FPF) of less than 5.6 microns of at least 45%;
(ii) a FPF of less than 3.4 microns of at least 30%; or a FPF of less than 5.0 microns of at least 45%; or
(iii) a mass median aerodynamic diameter (MMAD) of between about 1 micron and about 5 microns.

22-24. (canceled)

25. A method for treating a migraine or a symptom thereof, comprising administering to a subject in need thereof via oral inhalation an effective amount of a dry powder, wherein the dry powder comprises respirable dry particles that comprise dihydroergotamine (DHE) or a salt, hydrate, or polymorph thereof; a monovalent metal cation salt and one or more excipients.

26-27. (canceled)

28. The method of claim 25, wherein the dry powder is administered to the subject at any point during a migraine.

29-31. (canceled)

32. The method of claim 25, wherein the Cₘₐₓ of DHE in the subject after administering the dry powder is between about 3000 pg/mL and about 5000 pg/mL.

33. The method of claim 25, wherein the Tₘₐₓ of DHE in the subject after administering the dry powder is less than about 5 minutes.

34-35. (canceled)

36. The method of claim 25, wherein the AUC₀₋₄₈ₕ of DHE in the subject after administering the dry powder is between about 4500 pg*h/mL and about 9500 pg*h/mL.

37. The method of claim 25, wherein a total dose of DHE or a salt, hydrate, or polymorph thereof of between about 0.5 mg to about 2.0 mg is administered to the subject.

38. A receptacle comprising a dry powder, wherein the dry powder comprises respirable dry particles that comprise dihydroergotamine (DHE) or a salt, hydrate, or polymorph thereof; a monovalent metal cation salt and one or more excipients.

39. The receptacle of claim 38, wherein the receptacle contains about 20 mg of the dry powder or less.

40. The receptacle of claim 38, wherein the receptacle contains a nominal dose of DHE or a salt, hydrate, or polymorph thereof of between about 100 μg and about 2000 μg.

41. A dry powder inhaler (DPI) that contains a dry powder, wherein the dry powder comprises respirable dry particles that comprise dihydroergotamine (DHE) or a salt, hydrate, or polymorph thereof, a monovalent metal cation salt and one or more excipients.

42. (canceled)
